SEQUENCE LISTING (134 sequences)

SEQ ID NO: 1 - RNA, 67 nt, Streptococcus pyogenes
agcauagcaaguuaaaauaaggcuaguccguuaucaacuugaaaaaguggcaccgagucggugcuuu

SEQ ID NO: 2 - RNA, 16 nt, Streptococcus pyogenes
guuuuagagcuaugcu

SEQ ID NO: 3 - RNA, 36 nt, Artificial Sequence
  Synthesized
uggauggaggaaugaggaguguuuuagagcuaugcu

SEQ ID NO: 4 - RNA, 36 nt, Artificial Sequence
  Synthesized
gaguccgagcagaagaagaaguuuuagagcuaugcu

SEQ ID NO: 5 - RNA, 36 nt, Streptococcus pyogenes; identical to SEQ ID NO: 3

SEQ ID NO: 6 - RNA, 36 nt, Streptococcus pyogenes; identical to SEQ ID NO: 4

SEQ ID NO: 7 - DNA, 47 bp, Homo sapiens
gccgaagcttcttggatggaggaatgaggagtgggcttctagaggcc

SEQ ID NO: 8 - DNA, 47 bp, Homo sapiens
ggcctctagaagcccactcctcattcctccatccaagaagcttcggc

SEQ ID NO: 9 - DNA, 47 bp, Homo sapiens
gccgaagcttcttggatggaggaatgaggagagggcttctagaggcc

SEQ ID NO: 10 - DNA, 47 bp, Homo sapiens
ggcctctagaagccctctcctcattcctccatccaagaagcttcggc

SEQ ID NO: 11 - DNA, 47 bp, Homo sapiens
gccgaagcttcttggatggaggaatgaggggtgggcttctagaggcc

SEQ ID NO: 12 - DNA, 47 bp, Homo sapiens
ggcctctagaagcccacccctcattcctccatccaagaagcttcggc

SEQ ID NO: 13 - DNA, 47 bp, Homo sapiens
gccgaagcttcttggatggaggaatgacgagtgggcttctagaggcc

SEQ ID NO: 14 - DNA, 47 bp, Homo sapiens
ggcctctagaagcccactcgtcattcctccatccaagaagcttcggc

SEQ ID NO: 15 - DNA, 47 bp, Homo sapiens
gccgaagcttcttggatggaggaaagaggagtgggcttctagaggcc

SEQ ID NO: 16 - DNA, 47 bp, Homo sapiens
ggcctctagaagcccactcctctttcctccatccaagaagcttcggc

SEQ ID NO: 17 - DNA, 47 bp, Homo sapiens
gccgaagcttcttggatggaggactgaggagtgggcttctagaggcc

SEQ ID NO: 18 - DNA, 47 bp, Homo sapiens
ggcctctagaagcccactcctcagtcctccatccaagaagcttcggc

SEQ ID NO: 19 - DNA, 47 bp, Homo sapiens
gccgaagcttcttggatggagaaatgaggagtgggcttctagaggcc

SEQ ID NO: 20 - DNA, 47 bp, Homo sapiens
ggcctctagaagcccactcctcatttctccatccaagaagcttcggc

SEQ ID NO: 21 - DNA, 47 bp, Homo sapiens
gccgaagcttcttggatggtggaatgaggagtgggcttctagaggcc

SEQ ID NO: 22 - DNA, 47 bp, Homo sapiens
ggcctctagaagcccactcctcattccaccatccaagaagcttcggc

SEQ ID NO: 23 - DNA, 47 bp, Homo sapiens
gccgaagcttcttggattgaggaatgaggagtgggcttctagaggcc

SEQ ID NO: 24 - DNA, 47 bp, Homo sapiens
ggcctctagaagcccactcctcattcctcaatccaagaagcttcggc

SEQ ID NO: 25 - DNA, 47 bp, Homo sapiens
gccgaagcttcttgggtggaggaatgaggagtgggcttctagaggcc

SEQ ID NO: 26 - DNA, 47 bp, Homo sapiens
ggcctctagaagcccactcctcattcctccacccaagaagcttcggc

SEQ ID NO: 27 - DNA, 47 bp, Homo sapiens
gccgaagcttcttcgatggaggaatgaggagtgggcttctagaggcc

SEQ ID NO: 28 - DNA, 47 bp, Homo sapiens
ggcctctagaagcccactcctcattcctccatcgaagaagcttcggc

SEQ ID NO: 29 - DNA, 47 bp, Homo sapiens
gccgaagcttctaggatggaggaatgaggagtgggcttctagaggcc

SEQ ID NO: 30 - DNA, 47 bp, Homo sapiens
ggcctctagaagcccactcctcattcctccatcctagaagcttcggc

SEQ ID NO: 31 - DNA, 47 bp, Homo sapiens
gccgaagcttctgagtccgagcagaagaagaagggcttctagaggcc

SEQ ID NO: 32 - DNA, 47 bp, Homo sapiens
ggcctctagaagcccttcttcttctgctcggactcagaagcttcggc

SEQ ID NO: 33 - DNA, 47 bp, Homo sapiens
gccgaagcttctgagtccgagcagaagaagaggggcttctagaggcc

SEQ ID NO: 34 - DNA, 47 bp, Homo sapiens
ggcctctagaagcccctcttcttctgctcggactcagaagcttcggc

SEQ ID NO: 35 - DNA, 47 bp, Homo sapiens
gccgaagcttctgagtccgagcagaagaataagggcttctagaggcc

SEQ ID NO: 36 - DNA, 47 bp, Homo sapiens
ggcctctagaagcccttattcttctgctcggactcagaagcttcggc

SEQ ID NO: 37 - DNA, 47 bp, Homo sapiens
gccgaagcttctgagtccgagcagaaggagaagggcttctagaggcc

SEQ ID NO: 38 - DNA, 47 bp, Homo sapiens
ggcctctagaagcccttctccttctgctcggactcagaagcttcggc

SEQ ID NO: 39 - DNA, 47 bp, Homo sapiens
gccgaagcttctgagtccgagcagaggaagaagggcttctagaggcc

SEQ ID NO: 40 - DNA, 47 bp, Homo sapiens
ggcctctagaagcccttcttcctctgctcggactcagaagcttcggc

SEQ ID NO: 41 - DNA, 47 bp, Homo sapiens
gccgaagcttctgagtccgggcagaagaagaagggcttctagaggcc

SEQ ID NO: 42 - DNA, 47 bp, Homo sapiens
ggcctctagaagcccttcttcttctgcccggactcagaagcttcggc

SEQ ID NO: 43 - DNA, 47 bp, Homo sapiens
gccgaagcttctgagtccgagtagaagaagaagggcttctagaggcc

SEQ ID NO: 44 - DNA, 47 bp, Homo sapiens
ggcctctagaagcccttcttcttctactcggactcagaagcttcggc

SEQ ID NO: 45 - DNA, 47 bp, Homo sapiens; identical to SEQ ID NO: 41

SEQ ID NO: 46 - DNA, 47 bp, Homo sapiens; identical to SEQ ID NO: 42

SEQ ID NO: 47 - DNA, 47 bp, Homo sapiens
gccgaagcttctgagtctgagcagaagaagaagggcttctagaggcc

SEQ ID NO: 48 - DNA, 47 bp, Homo sapiens
ggcctctagaagcccttcttcttctgctcagactcagaagcttcggc

SEQ ID NO: 49 - DNA, 47 bp, Homo sapiens
gccgaagcttctgaggccgagcagaagaagaagggcttctagaggcc

SEQ ID NO: 50 - DNA, 47 bp, Homo sapiens
ggcctctagaagcccttcttcttctgctcggcctcagaagcttcggc

SEQ ID NO: 51 - DNA, 47 bp, Homo sapiens
gccgaagcttctgcgtccgagcagaagaagaagggcttctagaggcc

SEQ ID NO: 52 - DNA, 47 bp, Homo sapiens
ggcctctagaagcccttcttcttctgctcggacgcagaagcttcggc

SEQ ID NO: 53 - DNA, 47 bp, Homo sapiens
gccgaagcttcttagtccgagcagaagaagaagggcttctagaggcc

SEQ ID NO: 54 - DNA, 47 bp, Homo sapiens
ggcctctagaagcccttcttcttctgctcggactaagaagcttcggc

SEQ ID NO: 55 - DNA, 26 bp, Escherichia coli
gcgacacggaaatgttgaatactcat

SEQ ID NO: 56 - DNA, 18 bp, Escherichia coli
cagcgagtcagtgagcga

SEQ ID NO: 57 - DNA, 52 bp, Homo sapiens
  misc_feature (7)..(10): n is a, c, g, or t
  misc_feature (13)..(13): n is a, c, g, or t
  misc_feature (34)..(37): n is a, c, g, or t
aacacannnnccnactcctcattcctccatccannnnacctgccgagaacac

SEQ ID NO: 58 - DNA, 53 bp, Homo sapiens
  misc_feature (7)..(10): n is a, c, g, or t
  misc_feature (13)..(13): n is a, c, g, or t
  misc_feature (34)..(37): n is a, c, g, or t
tcttctnnnnccnttcttcttctgctcggactcnnnnacctgccgagtcttct

SEQ ID NO: 59 - DNA, 63 bp, Homo sapiens
aatgatacggcgaccaccgagatctacactctttccctacacgacgctcttccgatctactgt

SEQ ID NO: 60 - DNA, 63 bp, Homo sapiens
aatgatacggcgaccaccgagatctacactctttccctacacgacgctcttccgatctctgaa

SEQ ID NO: 61 - DNA, 63 bp, Homo sapiens
aatgatacggcgaccaccgagatctacactctttccctacacgacgctcttccgatcttgact

SEQ ID NO: 62 - DNA, 63 bp, Homo sapiens
aatgatacggcgaccaccgagatctacactctttccctacacgacgctcttccgatcttgcaa

SEQ ID NO: 63 - DNA, 63 bp, Homo sapiens
aatgatacggcgaccaccgagatctacactctttccctacacgacgctcttccgatctgcatt

SEQ ID NO: 64 - DNA, 63 bp, Homo sapiens
aatgatacggcgaccaccgagatctacactctttccctacacgacgctcttccgatctcatga

SEQ ID NO: 65 - DNA, 63 bp, Homo sapiens
aatgatacggcgaccaccgagatctacactctttccctacacgacgctcttccgatctatgct

SEQ ID NO: 66 - DNA, 63 bp, Homo sapiens
aatgatacggcgaccaccgagatctacactctttccctacacgacgctcttccgatctctagt

SEQ ID NO: 67 - DNA, 63 bp, Homo sapiens
aatgatacggcgaccaccgagatctacactctttccctacacgacgctcttccgatctgctaa

SEQ ID NO: 68 - DNA, 63 bp, Homo sapiens
aatgatacggcgaccaccgagatctacactctttccctacacgacgctcttccgatctcagtt

SEQ ID NO: 69 - DNA, 63 bp, Homo sapiens
aatgatacggcgaccaccgagatctacactctttccctacacgacgctcttccgatctgtcat

SEQ ID NO: 70 - DNA, 63 bp, Homo sapiens
aatgatacggcgaccaccgagatctacactctttccctacacgacgctcttccgatctacgta

SEQ ID NO: 71 - DNA, 50 bp, Homo sapiens
acagtagatcggaagagcgtcgtgtagggaaagagtgtagatctcggtgg

SEQ ID NO: 72 - DNA, 50 bp, Homo sapiens
ttcagagatcggaagagcgtcgtgtagggaaagagtgtagatctcggtgg

SEQ ID NO: 73 - DNA, 50 bp, Homo sapiens
agtcaagatcggaagagcgtcgtgtagggaaagagtgtagatctcggtgg

SEQ ID NO: 74 - DNA, 50 bp, Homo sapiens
ttgcaagatcggaagagcgtcgtgtagggaaagagtgtagatctcggtgg

SEQ ID NO: 75 - DNA, 50 bp, Homo sapiens
aatgcagatcggaagagcgtcgtgtagggaaagagtgtagatctcggtgg

SEQ ID NO: 76 - DNA, 50 bp, Homo sapiens
tcatgagatcggaagagcgtcgtgtagggaaagagtgtagatctcggtgg

SEQ ID NO: 77 - DNA, 50 bp, Homo sapiens
agcatagatcggaagagcgtcgtgtagggaaagagtgtagatctcggtgg

SEQ ID NO: 78 - DNA, 50 bp, Homo sapiens
actagagatcggaagagcgtcgtgtagggaaagagtgtagatctcggtgg

SEQ ID NO: 79 - DNA, 50 bp, Homo sapiens
ttagcagatcggaagagcgtcgtgtagggaaagagtgtagatctcggtgg

SEQ ID NO: 80 - DNA, 50 bp, Homo sapiens
aactgagatcggaagagcgtcgtgtagggaaagagtgtagatctcggtgg

SEQ ID NO: 81 - DNA, 50 bp, Homo sapiens
atgacagatcggaagagcgtcgtgtagggaaagagtgtagatctcggtgg

SEQ ID NO: 82 - DNA, 50 bp, Homo sapiens
tacgtagatcggaagagcgtcgtgtagggaaagagtgtagatctcggtgg

SEQ ID NO: 83 - DNA, 20 bp, Homo sapiens
aatgatacggcgaccaccga

SEQ ID NO: 84 - DNA, 40 bp, Homo sapiens
caagcagaagacggcatacgagatacctgccgagaacaca

SEQ ID NO: 85 - DNA, 40 bp, Homo sapiens
caagcagaagacggcatacgagatacctgccgagtcttct

SEQ ID NO: 86 - DNA, 15 bp, Homo sapiens
gacggcatacgagat

SEQ ID NO: 87 - DNA, 28 bp, Homo sapiens
aacaatctcgtatgccgtcttctgcttg

SEQ ID NO: 88 - DNA, 28 bp, Homo sapiens
tcttatctcgtatgccgtcttctgcttg

SEQ ID NO: 89 - DNA, 82 bp, Homo sapiens
  misc_feature (59)..(62): n is a, c, g, or t
aatgatacggcgaccaccgagatctacactctttccctacacgacgctcttccgatctnnnnacctacctgccgagaacaca

SEQ ID NO: 90 - DNA, 82 bp, Homo sapiens
  misc_feature (59)..(62): n is a, c, g, or t
aatgatacggcgaccaccgagatctacactctttccctacacgacgctcttccgatctnnnnacctacctgccgagtcttct

SEQ ID NO: 91 - DNA, 24 bp, Homo sapiens
caagcagaagacggcatacgagat

SEQ ID NO: 92 - RNA, 36 nt, Homo sapiens; identical to SEQ ID NO: 4

SEQ ID NO: 93 - RNA, 36 nt, Homo sapiens; identical to SEQ ID NO: 4

SEQ ID NO: 94 - RNA, 36 nt, Homo sapiens; identical to SEQ ID NO: 4

SEQ ID NO: 95 - RNA, 36 nt, Homo sapiens; identical to SEQ ID NO: 4

SEQ ID NO: 96 - RNA, 36 nt, Homo sapiens; identical to SEQ ID NO: 4

SEQ ID NO: 97 - RNA, 36 nt, Homo sapiens; identical to SEQ ID NO: 4

SEQ ID NO: 98 - RNA, 36 nt, Homo sapiens; identical to SEQ ID NO: 4

SEQ ID NO: 99 - RNA, 36 nt, Homo sapiens; identical to SEQ ID NO: 4

SEQ ID NO: 100 - RNA, 36 nt, Homo sapiens; identical to SEQ ID NO: 4

SEQ ID NO: 101 - RNA, 36 nt, Homo sapiens; identical to SEQ ID NO: 4

SEQ ID NO: 102 - RNA, 36 nt, Homo sapiens; identical to SEQ ID NO: 4

SEQ ID NO: 103 - RNA, 36 nt, Homo sapiens; identical to SEQ ID NO: 4

SEQ ID NO: 104 - RNA, 36 nt, Homo sapiens; identical to SEQ ID NO: 4

SEQ ID NO: 105 - RNA, 36 nt, Homo sapiens; identical to SEQ ID NO: 4

SEQ ID NO: 106 - RNA, 36 nt, Homo sapiens; identical to SEQ ID NO: 4

SEQ ID NO: 107 - RNA, 36 nt, Homo sapiens; identical to SEQ ID NO: 4

SEQ ID NO: 108 - RNA, 36 nt, Homo sapiens; identical to SEQ ID NO: 4

SEQ ID NO: 109 - RNA, 36 nt, Homo sapiens; identical to SEQ ID NO: 4

SEQ ID NO: 110 - RNA, 36 nt, Homo sapiens; identical to SEQ ID NO: 4

SEQ ID NO: 111 - RNA, 36 nt, Homo sapiens; identical to SEQ ID NO: 4

SEQ ID NO: 112 - RNA, 36 nt, Homo sapiens; identical to SEQ ID NO: 4

SEQ ID NO: 113 - RNA, 36 nt, Homo sapiens; identical to SEQ ID NO: 4

SEQ ID NO: 114 - RNA, 36 nt, Homo sapiens; identical to SEQ ID NO: 4

SEQ ID NO: 115 - RNA, 36 nt, Homo sapiens; identical to SEQ ID NO: 4

SEQ ID NO: 116 - RNA, 36 nt, Homo sapiens; identical to SEQ ID NO: 4

SEQ ID NO: 117 - RNA, 36 nt, Homo sapiens; identical to SEQ ID NO: 4

SEQ ID NO: 118 - RNA, 36 nt, Homo sapiens; identical to SEQ ID NO: 4

SEQ ID NO: 119 - RNA, 36 nt, Homo sapiens; identical to SEQ ID NO: 4

SEQ ID NO: 120 - RNA, 36 nt, Homo sapiens; identical to SEQ ID NO: 4

SEQ ID NO: 121 - RNA, 36 nt, Homo sapiens; identical to SEQ ID NO: 4

SEQ ID NO: 122 - RNA, 36 nt, Homo sapiens; identical to SEQ ID NO: 4

SEQ ID NO: 123 - RNA, 36 nt, Homo sapiens; identical to SEQ ID NO: 4

SEQ ID NO: 124 - RNA, 36 nt, Homo sapiens; identical to SEQ ID NO: 4

SEQ ID NO: 125 - RNA, 36 nt, Homo sapiens; identical to SEQ ID NO: 4

SEQ ID NO: 126 - RNA, 36 nt, Homo sapiens; identical to SEQ ID NO: 4

SEQ ID NO: 127 - RNA, 36 nt, Homo sapiens; identical to SEQ ID NO: 4

SEQ ID NO: 128 - RNA, 36 nt, Homo sapiens; identical to SEQ ID NO: 4

SEQ ID NO: 129 - RNA, 36 nt, Homo sapiens; identical to SEQ ID NO: 4

SEQ ID NO: 130 - RNA, 36 nt, Homo sapiens; identical to SEQ ID NO: 4

SEQ ID NO: 131 - RNA, 36 nt, Homo sapiens; identical to SEQ ID NO: 4

SEQ ID NO: 132 - DNA, 36 bp, Homo sapiens
gagtccgagcagaagaagaaguuuuagagcuaugcu

SEQ ID NO: 133 - DNA, 36 bp, Homo sapiens; identical to SEQ ID NO: 132

SEQ ID NO: 134 - DNA, 36 bp, Homo sapiens; identical to SEQ ID NO: 132

(19) United States
(12) Patent Application Publication
(10) Pub. No.: US 2021/0277392 A1
(43) Pub. Date: Sep. 9, 2021

(54) CHEMICALLY-MODIFIED GUIDE RNAS TO IMPROVE CRISPR-CAS PROTEIN SPECIFICITY

(71) Applicant: THE GOVERNORS OF THE UNIVERSITY OF ALBERTA, Edmonton, CA (US)

(72) Inventors: Basil HUBBARD, Edmonton, Alberta (CA); Christopher CROMWELL, Edmonton, Alberta (CA)

(21) Appl. No.: 16/760,868

(22) PCT Filed: Nov. 2, 2018
(86) PCT No.: PCT/CA2018/000210

§ 371 Date: Apr. 30, 2020

Related U.S. Application Data
(60) Provisional application No. 62/580,873, filed on Nov. 2, 2017.

(51) Int. Cl. C12N 15/11; C12N 9/22; C12N 15/62

(52) U.S. Cl. CPC C12N 15/111; C12N 9/22; C12N 15/62; C12N 2310/20; C12N 2310/3231; C12N 2310/344; C12N 2310/312; C12N 2320/51; C12N 2310/321

(57) ABSTRACT

A method of increasing specificity of binding of a CRISPR-Cas protein-guide RNA complex to a selected target nucleic acid sequence is provided. The method comprises contacting a nucleic acid molecule comprising the selected target nucleic acid sequence with the complex comprising the CRISPR-Cas protein and the guide RNA, wherein the guide RNA comprises a complementarity region at the 5′ end of the guide RNA that binds to a complementary strand of the selected target nucleic acid sequence, wherein the guide RNA comprises at least one modified nucleic acid within the complementarity region; wherein the guide RNA complementarity region binds and directs the CRISPR-Cas protein (e.g. CRISPR/Cas9) to the selected target nucleic acid sequence, thereby increasing specificity of binding of the CRISPR-Cas protein-guide RNA complex to the selected target nucleic acid sequence. The modified nucleic acid may be a bridged nucleic acid, a deoxyribonucleic acid, or a 2-0-methyl RNA phosphonoacetate-modified crRNA, or a functional equivalent that improves specificity by inducing similar conformational changes in the CRISPR-Cas system. Guide RNAs, kits comprising a guide RNA together with a CRISPR-Cas protein, and complexes comprising a guide RNA and a CRISPR-Cas proteins are also provided.

### FIELD

The present application pertains to the field of molecular biology. More particularly, the present application relates to the use of bridged nucleotide-modified guide RNAs to improve CRISPR-Cas protein specificity, including gene editing specificity.

### BACKGROUND

DNA-binding proteins carry out a number of functions, including organizing and packaging DNA, protecting DNA from damage, and functionalizing the information encoded in DNA. These proteins can form two types of interactions with DNA: 1) non-specific (binding to random nucleotides) and 2) sequence-specific. Non-specific DNA binding proteins are often used to provide structural support and in repair. Examples, which include primarily structural roles, include histones and HMG proteins.

The second class of DNA-binding domains constitute domain structures which have evolved to read DNA in a more precise manner. These are typically used to initiate transcription in a selective manner. Examples of these include MyoD—which recognizes E-box sequences using a basic helix loop helix DNA binding domain; Zif268—a prototypical zinc finger (ZF), which contains a zinc finger domain that recognizes a nucleotide triplet sequence; and arrays of transcription activator like effector (TALE) domains, which consist of a series of approximately 33 amino acid repeats that each specify recognition of one DNA base using a simple code. Importantly, these last two groups can be ‘programmed’ to bind to virtually any DNA sequence. In the case of ZFs, this programming is not intuitive and usually requires protein directed evolution, while in the case of TALEs, simply changing two amino acids in each repeat according to a specific code can be used to designate its target sequence.

Earlier research has exploited the ability of ZFs and TALEs to be programmed to bind to any DNA sequence to design artificial transcription factors. A specific ZF or TALE array may be fused to an effector domain such as a transcriptional activation domain, repressor domain, or a histone modifying enzyme. These have been used in a wide variety of applications ranging from functional genomics (epigenetic engineering) to synthetic biology (gene circuits).

Another major application of engineered sequence-specific DNA binding proteins has been in the area of genome engineering. Through fusion of these DNA binding domains to nucleases or recombinases, a genome can be edited in a precise manner. For example, by attaching zinc finger arrays to subunits of the Fok1 endonuclease, a specific gene can be targeted for knockout by non-homologous end joining (NHEJ), or for editing through homologous recombination (HR) repair in the presence of a customized single-strand oligodeoxynucleotide (ssODN) or similar with flanking regions that are complementary to the target site.

Cas9 is a sequence-specific DNA binding protein that has been characterized and applied to many of these applications. Cas9 is unique because its DNA recognition is not mediated primarily through amino acid contacts (although some protein-DNA recognition does occur), but rather through RNA. This makes programming Cas9 to bind to a sequence of DNA much easier.

The Clustered Regularly Interspaced Short Palindromic Repeats (CRISPR)/Cas9 system, originally characterized as a prokaryotic immune system has become a widely-used tool for genome editing applications. This system has been applied in genetic studies ranging from yeast to mice, functional genomics screens and human therapeutic contexts. Two non-coding RNA elements direct sequence-specific DNA cleavage by the Cas9 system. The CRISPR-RNA (crRNA) contains a 20-bp RNA sequence complementary to the target DNA sequence, while the transactivating crRNA (tracrRNA) acts as a bridge between the crRNA and Cas9 enzyme. These RNA elements, which together form a guide RNA (gRNA), can be combined into a single chimeric RNA molecule called a single guide RNA (sgRNA). Target recognition proceeds through recognition of an upstream protospacer adjacent motif (PAM) (5′NGG′3 in S. pyogenes) on the target DNA strand, followed by DNA melting and hybridization of the first 10-12 bp of the 3′ end of crRNA sequence (seed pairing), and formation of an R-loop structure. Complete hybridization between the guide segment and target DNA drives conformational changes in the HNH and RuvC nuclease domains that result in DNA cleavage 3-bp upstream of the PAM. While mutations within the PAM sequence mostly abolish Cas9 cleavage activity, mutations within the target sequence may be permitted, resulting in cleavage of off-target DNA sequences.

Cas9 has several advantages over traditional programmable DNA binding domains. It does not require any difficult directed evolution or cloning to reprogram. Cas9 can be used with multiple gRNAs to simultaneously target multiple genes. As well, the specificity of Cas9 is reasonably good. At least for these reasons, it has been used to edit the genomes of a number of different organisms ranging from plants to mice. Moreover, the medical implications of this technology are potentially immense since it may be used to edit human gene defects associated with disease (e.g. sickle cell anemia).

The specificity of Cas9 is a critical issue when considering the possibility of using it for medical applications in humans, given that there are over 30 trillion cells in the body and that even a single off-target event in one of these cells could cause serious consequences.

Cas9 DNA cleavage specificity is highly dependent on the crRNA sequence and correlates with target-crRNA folding stability. A number of approaches have been deployed to improve off-target DNA cleavage by Cas9. These include engineering variants of Cas9 with diminished non-specific DNA interactions, as in the case of eSPCas9 and SpCas9-HF, a paired Cas9 nickase system, as well as delivery strategies displaying burst kinetics, such as Cas9 ribonucleoprotein (RNP) delivery. In addition, computational approaches have been developed to design sgRNAs with minimal off-target activity. Reducing the number of nucleotides in the spacer sequence from 20 to 17-18 bp (“tru-guides”) also improves specificity, but reduces on-target cleavage efficiency. Despite these advances, off-target cutting and generation of accessory mutations remains a significant barrier for clinical translation of Cas9.

Several studies have investigated the use of chemically-modified nucleic acids to improve crRNA nuclease-resistance.

Despite the developments, there exists a need for improving the specificity of the CRISPR/Cas9 system.

This background information is provided for the purpose of making known information believed by the applicant to be of possible relevance to the present invention. No admission is necessarily intended, nor should be construed, that any of the preceding information constitutes prior art against the present invention.

### SUMMARY

The present invention comprises a novel method which provides improved specificity of CRISPR-Cas protein systems, such as the Class 2 CRISPR/Cas9 system. More particularly, the method incorporates the use of modified nucleic acids, including bridged nucleotides including first-generation and next-generation bridged nucleotides, or other modified nucleotides, such as those which induce similar conformations in the enzyme, internally in guide RNAs, in particular crRNAs, to improve the specificity of CRISPR-Cas protein systems, such as the Class 2 CRISPR/Cas9 system.

In one aspect, there is provided a method of increasing specificity of binding of a CRISPR-Cas protein-guide RNA complex to a selected target nucleic acid sequence, the method comprising: contacting a target nucleic acid molecule comprising the selected target nucleic acid sequence with the complex comprising the CRISPR-Cas protein and the guide RNA, wherein the guide RNA comprises a complementarity region at the 5′ end of the guide RNA that binds to a complementary strand of the selected target nucleic acid sequence, wherein the guide RNA comprises at least one modified nucleic acid within the complementarity region; wherein the guide RNA complementarity region binds and directs the CRISPR-Cas protein to the selected target nucleic acid sequence, thereby increasing specificity of binding of the CRISPR-Cas protein-guide RNA complex to the selected target nucleic acid sequence. The modified nucleic acid may be a bridged nucleic acid. In other embodiments, the modified nucleotide may mimic the structural effects of a bridged nucleic acid, such as a deoxynucleotide, or 2′-O-methyl RNA phosphonoacetate nucleotide.

In another aspect, there is provided a guide RNA comprising a complementarity region at the 5′ end of the guide RNA that binds to a complementary strand of a selected target nucleic acid sequence within a nucleic acid molecule, wherein the guide RNA comprises at least one modified nucleic acid within the complementarity region.

In yet another aspect, there is provided a complex comprising: a CRISPR-Cas protein; and a guide RNA comprising a complementarity region at the 5′ end of the guide RNA that binds to a complementary strand of a selected target nucleic acid sequence within a nucleic acid molecule, wherein the guide RNA comprises at least one modified nucleic acid within the complementarity region.

In yet another aspect, there is provided a kit comprising the guide RNA as defined above complexed with a CRISPR-Cas protein; optionally with instructions for use.

In all cases, the CRISPR-Cas protein may be a class 2 CRISPR-Cas protein, such as a CRISPR-Cas9 protein.

### BRIEF DESCRIPTION OF THE FIGURES

For a better understanding of the present invention, as well as other aspects and further features thereof, reference is made to the following description which is to be used in conjunction with the accompanying drawings, where:

FIG. 1 illustrates the structures of RNA, 2′,4′-BNA (also referred to herein as “LNA”), and 2′,4′-BNA^{NC}[N-Me] (also referred to herein as “BNA”).

FIG. 2 illustrates on- and off-target sequences. Off-targets for both the WAS and EMX1 on-target were previously identified to be highly cleaved in cells by Cas9 (Wang X, et al. (2014) Unbiased detection of off-target cleavage by CRISPR-Cas9 and TALENs using integrase defective lentiviral vectors. Nat. Biotechnol. 33(2):175-178) (Kim D, et al. (2016) Genome-wide target specificities of CRISPR-Cas9 nucleases revealed by multiplex Digenome-seq. Genome Res. 26:405-415) Locations of mismatched nucleotide bases within off-target sequences are indicated by surrounding black boxes.

FIG. 3 illustrates sequences of unmodified, BNA- and LNA-modified crRNAs targeting the WAS locus used in this study. Modified nucleic acids are indicated with highlighting.

FIG. 4 illustrates sequences of unmodified, BNA- and LNA-modified crRNAs targeting the EMX1 locus used in this study. Modified nucleic acids are indicated with highlighting.

FIG. 5 illustrates results of in vitro cleavage assays using RNA-, BNA- and LNA-modified gRNAs for both WAS and EMX1 on on- and off-target sequences. Black indicates high levels of in vitro cleavage, while white indicates no observable cleavage. As shown in both cases, the use of BNA- or LNA-modified gRNAs increases the specificity of Cas9. 150 nM Cas9 and 150 nM gRNA (high dose) were pre-incubated for 10 min at 37° C. before addition of on- or off-target DNA to a final concentration of 5 nM. Reactions were allowed to proceed for 1 h at 37° C. before purification via MinElute Purification Kit (Qiagen). Cleavage products were resolved on a 1% TBE agarose gel and processed with ImageJ.

FIG. 6 illustrates results of in vitro cleavage assays using RNA-, BNA- and LNA-gRNAs for both WAS and EMX1 on on- and off-target sequences. Black indicates high levels of in vitro cleavage, while white indicates no observable cleavage. As shown in both cases, the use of BNA- or LNA-modified gRNAs increases the specificity of Cas9. 15 nM Cas9 and 15 nM gRNA (low dose) were pre-incubated for 10 min at 37° C. before addition of on- or off-target DNA to a final concentration of 5 nM. Reactions were allowed to proceed for 1 h at 37° C. before purification via MinElute Purification Kit (Qiagen). Cleavage products were resolved on a 1% TBE agarose gel and processed with ImageJ.

FIG. 7 illustrates results of in vitro cleavage assays using RNA-, BNA- and LNA-modified gRNAs to test in vitro cleavage of off-target single nucleotide polymorphisms (SNPs) derived from the WAS gene sequence. Black indicates high levels of in vitro cleavage, while white indicates no observable cleavage. As shown in both cases, the use of BNA- or LNA-modified gRNAs increases the SNP specificity of Cas9. 15 nM Cas9 and 15 nM gRNA were pre-incubated for 10 min at 37° C. before addition of on- or off-target DNA to a final concentration of 5 nM. Reactions were allowed to proceed for 1 h at 37° C. before purification via MinElute Purification Kit (Qiagen). Cleavage products were resolved on a 1% TBE agarose gel and processed with ImageJ.

FIG. 8 illustrates results of in vitro cleavage assays using RNA-, BNA- and LNA-gRNAs to test in vitro cleavage of off-target SNPs derived from the EMX1 gene sequence. Black indicates high levels of in vitro cleavage, while white indicates no observable cleavage. As shown in both cases, the use of BNA- or LNA-modified gRNAs increases the SNP specificity of Cas9. 15 nM Cas9 and 15 nM gRNA were pre-incubated for 10 min at 37° C. before addition of on- or off-target DNA to a final concentration of 5 nM. Reactions were allowed to proceed for 1 h at 37° C. before purification via MinElute Purification Kit (Qiagen). Cleavage products were resolved on a 1% TBE agarose gel and processed with ImageJ.

FIG. 9 illustrates BNA-modified gRNAs complexed with either wild-type Cas9 or modified eSpCas9 and assayed for their respective DNA cleavage specificities on WAS on- and off-target sequences. This demonstrates the compatibility of BNA/LNA modified-gRNAs with modified Cas9 nucleases such as eSpCas9. In addition it demonstrates synergy and an additive quality in terms of specificity between the two technologies. Black indicates high levels of in vitro cleavage, while white indicates no detectable in vitro cleavage. 15 nM Cas9 and 15 nM gRNA were pre-incubated for 10 min at 37° C. before addition of on- or off-target DNA to a final concentration of 5 nM. Reactions were allowed to proceed for 1 h at 37° C. before purification via MinElute Purification Kit (Qiagen). Cleavage products were resolved on a 1% TBE agarose gel and processed with ImageJ.

FIG. 10A illustrates cellular cleavage assays using T7 endonuclease digestion assay (T7E) that reveals improved specificity of the BNA-modified gRNA targeting the WAS locus in cells. The image on the left shows the results of the T7E1 digestion assay, with % indel values shown underneath each lane. Graphical representation of the data on the right outlines the reduction in off-target cleavage after using the BNA-modified gRNA. U2OS-Cas9 cells (cells stably expressing Cas9) were transfected with gRNA to a final concentration of 30 nM using RNAiMAX (Thermo Fisher). Cells were incubated at 37° C. for 48 h before gDNA isolation via DNeasy Blood & Tissue Kit (Qiagen). 100 ng of isolated gDNA was used as template for PCR amplification of each target site using Q5 Hot Start High Fidelity DNA Polymerase (NEB). T7 Endonuclease I (NEB) digestion was performed as described by the manufacturer. Reaction products were resolved on a 2.5% TBE agarose gel, imaged with an Amersham Imager 600 (GE) and quantified through ImageJ. Asterisks denote non-specific T7E1 cleavage.

FIG. 10B illustrates results of targeted next-generation sequencing showing that off-target activity for the EMX1-directed gRNA is eliminated with use of a BNA-modified gRNA (while on-target activity is only slightly reduced). U2OS-Cas9 cells were transfected with gRNA to a final concentration of 30 nM using RNAiMAX (Thermo Fisher). Cells were incubated at 37° C. for 48 h before gDNA isolation via DNeasy Blood & Tissue Kit (Qiagen). 100 ng of isolated gDNA was used as template for PCR amplification of each target site using Q5 Hot Start High Fidelity DNA Polymerase (NEB). Illumina compatible indices were added in a second round of PCR followed by paired-end sequencing on an Illumina MiSeq.

FIG. 10C illustrates results of targeted next-generation sequencing showing that overall off-target activity for the WAS-directed gRNA is greatly improved with use of a BNA-modified gRNA (while on-target activity is only moderately reduced). U2OS-Cas9 cells were transfected with gRNA to a final concentration of 30 nM using RNAiMAX (Thermo Fisher). Cells were incubated at 37° C. for 48 h before gDNA isolation via DNeasy Blood & Tissue Kit (Qiagen). 100 ng of isolated gDNA was used as template for PCR amplification of each target site using Q5 Hot Start High Fidelity DNA Polymerase (NEB). Illumina compatible indices were added in a second round of PCR followed by paired-end sequencing on an Illumina MiSeq.

FIG. 11 illustrates a titration of target DNA (WAS target) on in vitro cleavage using fixed amounts of Cas9 and gRNA. This shows that overall in vitro on-target DNA affinity using unmodified, LNA-, and BNA-modified is relatively equivalent. 2.5 nM Cas9 and 2.5 nM gRNA were incubated at room temperature for 10 min prior to addition of DNA. Reactions were allowed to proceed for 1 h at 37° C. before purification via MinElute Purification Kit (Qiagen). Cleavage products were resolved on a 1% TBE agarose gel and processed with ImageJ.

FIG. 12 illustrates a titration of off-target DNA (WAS OT-3) on in vitro cleavage using fixed amounts of Cas9 and gRNA. This confirms the improved specificity of LNA/BNA-modified gRNAs in vitro, overall a wide range of DNA substrate concentrations. 2.5 nM Cas9 and 2.5 nM gRNA were incubated at room temperature for 10 min prior to addition of DNA. Reactions were allowed to proceed for 1 h at 37° C. before purification via MinElute Purification Kit (Qiagen). Cleavage products were resolved on a 1% TBE agarose gel and processed with ImageJ.

FIG. 13 illustrates gRNA titrations with fixed Cas9 concentrations on WAS on-target DNA substrate. This shows that modified gRNAs have similar affinity to unmodified ones for Cas9. Various amounts of gRNA were incubated with 15 nM Cas9 at room temperature for 10 min prior to addition of DNA. Reactions were allowed to proceed for 1 h at 37° C. before purification via MinElute Purification Kit (Qiagen). Cleavage products were resolved on a 1% TBE agarose gel and processed with ImageJ.

FIG. 14 illustrates gRNA titrations with fixed Cas9 concentrations on a WAS off-target DNA substrate (WAS-OT3). Titrating amounts of gRNA were incubated with 15 nM Cas9 at room temperature for 10 min prior to addition of DNA. Reactions were allowed to proceed for 1 h at 37° C. before purification via MinElute Purification Kit (Qiagen). Cleavage products were resolved on a 1% TBE agarose gel and processed with ImageJ.

FIG. 15 illustrates Cas9 RNP titrations using either modified or unmodified gRNAs on the WAS-on target DNA sequence (in vitro assay). This data shows that basal on target activity of RNP complexes incorporating either unmodified or LNA/BNA-modified gRNAs are equivalent (no reduction in complex activity in vitro). 80 nM Cas9 and 80 nM gRNA were incubated at room temperature for 10 min prior to addition of DNA. After Cas9 RNP assembly, decreasing titrations of the RNP solution were prepared, after which on- and off-target DNA was added to a final concentration of 5 nM. Reactions were allowed to proceed for 1 h at 37° C. before purification via MinElute Purification Kit (Qiagen). Cleavage products were resolved on a 1% TBE agarose gel and processed with ImageJ.

FIG. 16 illustrates Cas9 RNP titrations using either modified or unmodified gRNAs on a WAS-off target DNA sequence (in vitro assay, WAS-OT3). This data show that the specificity improvements are maintained even at high doses or RNP. 80 nM Cas9 and 80 nM gRNA were incubated at room temperature for 10 min prior to addition of DNA. After Cas9 RNP assembly, decreasing titrations of the RNP solution were prepared, after which on- and off-target DNA was added to a final concentration of 5 nM. Reactions were allowed to proceed for 1 h at 37° C. before purification via MinElute Purification Kit (Qiagen). Cleavage products were resolved on a 1% TBE agarose gel and processed with ImageJ.

FIG. 17 illustrates the effect of altering tracrRNA:crRNA ratios using either modified or unmodified gRNAs on DNA cleavage (WAS on-target). From this figure it can be inferred that modified-gRNAs complex with similar affinity and efficacy with unmodified ones to the tracRNA. To prepare gRNA, different ratios of tracrRNA to crRNA were prepared with the final concentration being 100 nM. After gRNA annealing, Cas9 and gRNA were complexed by incubating at room temperature for 10 min prior to addition of DNA. Reactions were allowed to proceed for 1 h at 37° C. before purification via MinElute Purification Kit (Qiagen). Cleavage products were resolved on a 1% TBE agarose gel and processed with ImageJ.

FIG. 18 illustrates the effect of altering tracrRNA:crRNA ratios using either modified or unmodified gRNAs on DNA cleavage (WAS off-target). From this figure it can be inferred that modified-gRNAs complex with similar affinity and efficacy with unmodified ones to the tracRNA. To prepare gRNA, different ratios of tracrRNA to crRNA were prepared with the final concentration being 100 nM. After gRNA annealing, Cas9 and gRNA were complexed by incubating at room temperature for 10 min prior to addition of DNA. Reactions were allowed to proceed for 1 h at 37° C. before purification via MinElute Purification Kit (Qiagen). Cleavage products were resolved on a 1% TBE agarose gel and processed with ImageJ.

FIG. 19 illustrates a Cas9 RNP EMSA using modified and unmodified gRNA and a 6-FAM labelled on target WAS probe. This figure shows that overall DNA-binding is unaltered by LNA/BNA gRNA modification. Equimolar amounts of dCas9 and gRNA were incubated for 10 min at room temperature to assemble active complexes. Titrating amounts of Cas9 RNP was added to 6-FAM labelled DNA target and incubated for 10 min at 37° C. Reactions were resolved on a 10% TBE polyacrylamide gel and imaged using a Typhoon Laser Gel Scanner (GE Healthcare).

FIG. 20 illustrates a Cas9 RNP EMSA using modified and unmodified gRNA and a 6-FAM labelled off-target WAS probe (WAS-OT-3). This figure shows that Cas9 can still bind in some capacity to off-target DNA sequences (that it doesn't cut), and that this facet of DNA binding is not altered by modification of the gRNA. Equimolar amounts of dCas9 and gRNA were incubated for 10 min at room temperature to assemble active complexes. Titrating amounts of Cas9 RNP was added to 6-FAM labelled DNA target and incubated for 10 min at 37° C. Reactions were resolved on a 10% TBE polyacrylamide gel and imaged using a Typhoon Laser Gel Scanner (GE Healthcare).

FIG. 21 (a) illustrates the assay used the measure the melting temperature of the naked heterduplex gRNA-DNA target, in the absence of Cas9; (b) illustrates the results of this assay. As shown in this figure, melting temperature was increased by incorporation of modified nucleic acids on both on- and off-target sequences.

FIG. 22 illustrates single-molecule (sm)FRET analysis of Cas9 complexed to on-target DNA in the presence of unmodified or BNA-modified gRNA. a) Scheme outlining the single-molecule fluorescent set-up used. b) Histogram outlining the population distribution of Cas9 molecules in either the low- or high-FRET state (low state corresponds to open conformation, high to fully zipped conformation). c) Representative trace showing the transition between high and low FRET energy states. d) Average dwell time of both low- and high-FRET states for unmodified and BNA-modified gRNAs. This data show that BNA-modification prevents transition of Cas9 into the fully-zipped conformation necessary for cleavage in the case of the off-target sequence, but allows it to proceed as normal in the case of the on-target WAS sequence.

FIG. 23 illustrates in vitro time course cleavage assays using WAS on and off-target sequences. 15 nM Cas9 and 15 nM gRNA were pre-complexed by incubating at room temperature for 10 min, after which on- and off-target DNA was added to a final concentration of 5 nM. Reactions were performed at 37° C. and stopped via MinElute Purification Kit (Qiagen). Cleavage products were resolved on a 1% TBE agarose gel and processed with ImageJ. This figure shows that Cas9 cleavage is slowed when using LNA/BNA modified gRNAs, as compared to the unmodified counterparts.

FIG. 24 illustrates in vitro time course cleavage assays using EMX1 on and off-target sequences. This figure shows that Cas9 cleavage is slowed when using LNA/BNA modified gRNAs, as compared to the unmodified counterparts (cleavage on off-target sequences is fully blocked—even after extended incubation periods). 15 nM Cas9 and 15 nM gRNA were pre-complexed by incubating at room temperature for 10 min, after which on- and off-target DNA was added to a final concentration of 5 nM. Reactions were performed at 37° C. and stopped via MinElute Purification Kit (Qiagen). Cleavage products were resolved on a 1% TBE agarose gel and processed with ImageJ.

FIG. 25 graphically illustrates the regions where incorporation of BNA into the gRNA showed the most dramatic effects on improving selectivity versus areas which had a lesser effect (or unable to determine).

FIG. 26 shows distributions of mutations for pre-selection (black) and post-selection (grey) libraries following in vitro high-throughput specificity profiling using either the WAS or EMX1 target sequence. In vitro selections were performed using 200 nM pre-selection library comprising ˜1012 potential off-target sequences and 1000 nM or 100 nM Cas9 complexed with 1000 nM or 100 nM unmodified (light grey) or BNA-modified (dark grey) gRNA. Pre- and post-selection libraries were subject to high-throughput sequencing on an Illumina MiSeq platform.

FIG. 27 shows heat maps showing DNA cleavage specificity scores following in vitro specificity profiling of ˜10¹² potential off-target sites using Cas9 complexed with unmodified (top) or BNA-modified (bottom) crRNAs targeting the WAS sequence (listed below each heatmap). Specificity scores of 1.0 (black) correspond to 100% enrichment for, while scores of −1.0 (white) correspond to 100% enrichment against a specific base-pair at a specific position along the target sequence. Black boxes denote the intended nucleotide at each position.

FIG. 28 shows heat maps showing DNA cleavage specificity scores following in vitro specificity profiling of ˜10¹² potential off-target sites using Cas9 complexed with unmodified (top) or BNA-modified (bottom) crRNAs targeting the EMX1 sequence (listed below each heatmap). Specificity scores of 1.0 (black) correspond to 100% enrichment for, while scores of −1.0 (white) correspond to 100% enrichment against a specific base-pair at a specific position along the target sequence. Black boxes denote the intended nucleotide at each position. Experiments shown were performed using 200 nM pre-selection library and 100 nM Cas9 RNP complex. Specificity scores were calculated using the formula: positive specificity score=(frequency of base pair at position[post-selection]−frequency of base pair at position[pre-selection])/(1−frequency of base pair at position[pre-selection]) and negative specificity score=(frequency of base pair at position[post-selection]−frequency of base pair at position[pre-selection])/(frequency of base pair at position[pre-selection]).

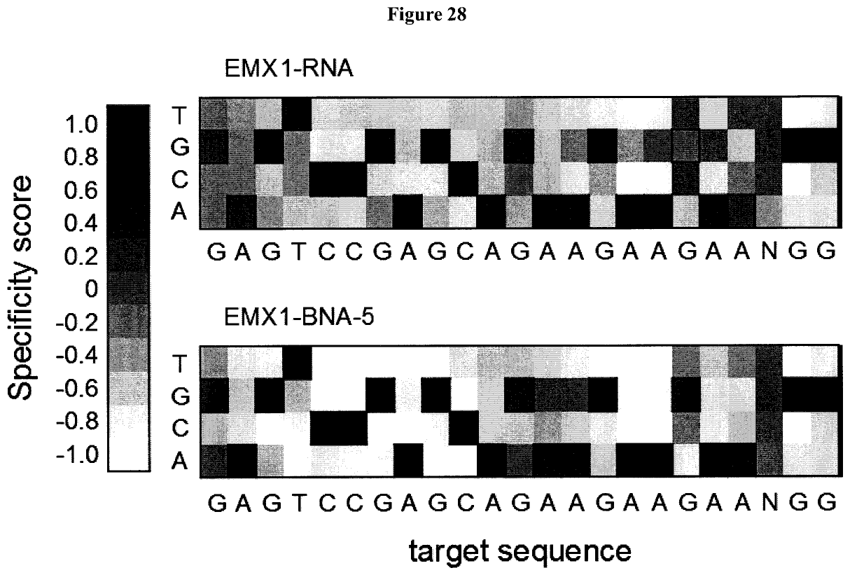

FIG. 29 shows a bar graph illustrating the quantitative differences in specificity score at each position along the 20 nucleotide Cas9 target site and 2 nucleotide PAM (of which of N of NGG is excluded) between the unmodified and BNA-modified crRNA for the WAS target.

FIG. 30 is a bar graph illustrating the quantitative differences in specificity score at each position along the 20 nucleotide Cas9 target site and 2 nucleotide PAM (of which of N of NGG is excluded) between the unmodified and BNA-modified crRNA for the EMX1 target.

FIG. 31 shows distributions of mutations are shown for pre-selection (black) and post-selection (grey) libraries following in vitro high-throughput specificity profiling using either the WAS or EMX1 target sequence.

FIG. 32 shows a heat map showing DNA cleavage specificity scores following in vitro specificity profiling of ˜10¹² potential off-target sites using Cas9 complexed with an LNA-modified crRNA targeting the WAS sequence (listed below the heat map).

FIG. 33 shows a heat map showing DNA cleavage specificity scores following in vitro specificity profiling of ˜10¹² potential off-target sites using Cas9 complexed with an LNA-modified crRNA targeting the EMX1 sequence (listed below the heat map).

FIG. 34 shows a bar graph illustrating the quantitative differences in specificity score at each position along the 20 nucleotide Cas9 target site and 2 nucleotide PAM (of which of N of NGG is excluded) between the unmodified and LNA-modified crRNA for the WAS target.

FIG. 35 shows a bar graph illustrating the quantitative differences in specificity score at each position along the 20 nucleotide Cas9 target site and 2 nucleotide PAM (of which of N of NGG is excluded) between the unmodified and LNA-modified crRNA for the EMX1 target.

FIG. 36 illustrates the structures of certain unmodified and modified nucleic acids: RNA, DNA, 2′-O-methyl RNA, 2′-O-methyl RNA phosphonoacetate, 2′,4′-BNA (also referred to herein as “LNA”) and 2′,4′-BNA^{NC}[NMe] (also referred to herein as “BNA”).

FIG. 37 shows a diagram outlining the sequences of unmodified and BNA-modified crRNAs targeting the EMX1 locus.

FIG. 38 shows a diagram outlining the sequences of unmodified and methyl RNA-modified crRNAs targeting the EMX1 locus used in this study.

FIG. 39 shows a diagram outlining the sequences of unmodified and 2′O methyl phosphonoacetate RNA-modified crRNAs targeting the EMX1 locus used in this study.

FIG. 40 shows a diagram outlining the sequences of unmodified and DNA-modified crRNAs targeting the EMX1 locus used in this study. DNA-modified nucleic acids are indicated by grey highlighting.

FIG. 41 shows heat maps showing DNA cleavage specificity scores following in vitro specificity profiling of ˜10¹² potential off-target sites using Cas9 complexed with BNA-modified crRNAs targeting the EMX1 sequence (listed below each heatmap).

FIG. 42 shows heat maps showing DNA cleavage specificity scores following in vitro specificity profiling of ˜10¹² potential off-target sites using Cas9 complexed with 2′-O-methyl RNA-modified crRNAs targeting the EMX1 sequence (listed below each heatmap).

FIG. 43 shows heat maps showing DNA cleavage specificity scores following in vitro specificity profiling of ˜10¹² potential off-target sites using Cas9 complexed with 2′-O-methyl RNA phosphonoacetate-modified crRNAs targeting the EMX1 sequence (listed below each heatmap).

FIG. 44 shows heat maps showing DNA cleavage specificity scores following in vitro specificity profiling of ˜10¹² potential off-target sites using Cas9 complexed with DNA-modified crRNAs targeting the EMX1 sequence (listed below each heatmap).

FIG. 45 shows bar graphs demonstrating the quantitative difference in specificity scores at each position along the 20 nucleotide target site and 2 nucleotide PAM (N of NGG excluded), between the unmodified and BNA-modified crRNA for EMX1 target sequences.

FIG. 46 shows bar graphs demonstrating the quantitative difference in specificity scores at each position along the 20 nucleotide target site and 2 nucleotide PAM (N of NGG excluded), between the unmodified and 2′-O-methyl RNA-modified crRNA for EMX1 target sequences.

FIG. 47 shows bar graphs demonstrating the quantitative difference in specificity scores at each position along the 20 nucleotide target site and 2 nucleotide PAM (N of NGG excluded), between the unmodified and 2′-O-methyl RNA phosphonoacetate-modified crRNA for EMX1 target sequences.

FIG. 48 shows bar graphs demonstrating the quantitative difference in specificity scores at each position along the 20 nucleotide target site and 2 nucleotide PAM (N of NGG excluded), between the unmodified and DNA-modified crRNA for EMX1 target sequences.

### DETAILED DESCRIPTION

Unless defined otherwise, all technical and scientific terms used herein have the same meaning as commonly understood by one of ordinary skill in the art to which this invention belongs.

Clustered regularly interspaced short palindromic repeats (CRISPR) and CRISPR-associated proteins (Cas) make up the CRISPR-Cas system. CRISPR systems have been divided into two major classes based on differences in their components and mechanisms of action. RNA-guided target cleavage in Class 1 systems (types I, III, and IV) requires a large complex of several effector proteins. In the Class 2 systems (type II, putative types V and VI), only one CRISPR-Cas protein, an RNA-guided endonuclease, for example Cas9 in type II and Cpf1 (CRISPR from Prevotella and Francisella-1) in type V, is required to mediate cleavage of invading genetic material (Wang, H. et al. (2016) CRISPR/Cas9 in Genome Editing and Beyond, Annu. Rev. Biochem. 85: 227-264, and references cited therein).

As used herein, the term “guide RNA” refers to the RNA that guides the CRISPR-Cas protein (or similar CRISPR/Cas system) to a selected target nucleic acid sequence on a target nucleic acid molecule, where the guide RNA hybridizes with and the CRISPR-Cas protein binds to, cleaves, or otherwise modulates the selected target nucleic acid sequence. The guide RNA may bear additional chemical modifications in addition to those described herein, including, but not limited to those described in: Hendel, A. et al. (2015) Chemically modified guide RNAs enhance CRISPR-Cas genome editing in human primary cells. Nat. Biotechnol. 33:985-989.

In some embodiments, there is provided a method of increasing specificity of binding of a CRISPR-Cas protein-guide RNA complex to a selected target nucleic acid sequence, the method comprising:
(a) contacting a nucleic acid molecule comprising the selected target nucleic acid sequence with the complex comprising the class 2 CRISPR-Cas protein and the guide RNA,
(b) wherein the guide RNA comprises a complementarity region at the 5′ end of the guide RNA that binds to a complementary strand of the selected target nucleic acid sequence,
(c) wherein the guide RNA complementarity region comprises at least one modified nucleic acid and wherein the guide RNA complementarity region binds and directs the class 2 CRISPR-Cas protein to the selected target nucleic acid sequence.

### CRISPR-Cas Proteins

The term “class 2 CRISPR-Cas protein” will be understood by those of skill in the art to refer to a Cas wild-type protein derived from a class 2 CRISPR-Cas system, homologs (e.g. orthologues) thereof, and variants (evolved or engineered) thereof. Variants of Cas proteins include, but are not limited to, Cas proteins that have been modified to reduce or eliminate nuclease activity (for example, a dCas9 or a dCpf1), Cas proteins that have been mutated in order to reduce off-target effects, and synthetic versions of Cas proteins, or versions of Cas9 with improved therapeutic or research purposes (improved nuclease resistance, smaller size, etc).

In some embodiments, the class 2 CRISPR-Cas protein comprises a Cas protein selected from Cas9, Cpfl, C2cl, C2c2 (also known as CRISPR-Cas effector Cas13a), and C2c3 proteins, and variants thereof.

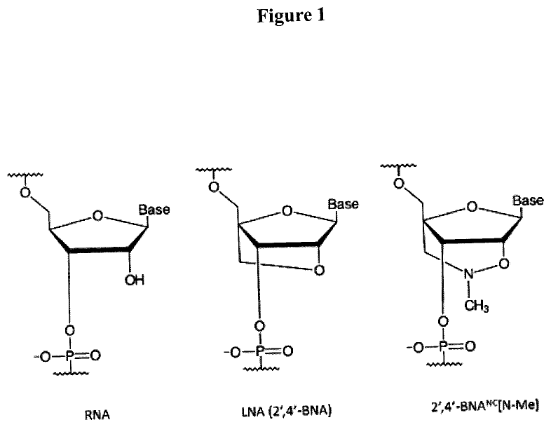

In some embodiments, the class 2 CRISPR-Cas protein is selected from a Cas9 protein, and variants thereof. A large variety of Cas9 proteins exist in different bacterial type II CRISPR systems. These Cas9 nucleases range from about 900 to 1,600 amino acids (AA) in three subclasses: type II-A, type II-B, and type II-C (see Wang, H. et al. (2016) CRISPR/Cas9 in Genome Editing and Beyond, Annu. Rev. Biochem. 85: 227-264, and references cited therein). Non-limiting exemplary species that the Cas9 protein may be from include Streptococcus pyogenes (Sp; the most commonly used Cas9 for genome engineering, having a simple PAM of NGG, or a weaker NAG, where N is any nucleotide), Staphylococcus aureus (Sa; with NNGRRT (where R is an A or G) as its PAM), Neisseria meningitidis (Nm; PAM=NNNNGATT), Streptococcus thermophilus 1 (St1; PAM=NNAGAAW, where W is an A or T), and other orthologues known to those of skill in the art—for example, Chylinski et al. classified Cas9 proteins from a large group of bacteria (RNA Biology (2013) 10:5, 1-12), and a large number of Cas9 proteins are listed in Supplementary FIG. 1 and Supplementary Table 1 thereof, as well as WO2017/173054 to INTELLIA THERAPEUTICS, INC. See also Ran, F. A, et al. Nature (2015) 520(7546): 186-191, including all Supplemental Data; Fonfara, I, et al. Nucleic Acids Research 42(4):2577-2590 (2014), including all Supplemental Data.

As used herein the term “variant” should be taken to mean the exhibition of qualities that have a pattern that deviates from what occurs in nature. The terms “non-naturally occurring” or “engineered” are used interchangeably and indicate the involvement of the hand of man. The terms, when referring to nucleic acid molecules or polypeptides mean that the nucleic acid molecule or the polypeptide is at least substantially free from at least one other component with which they are naturally associated in nature and as found in nature.

Variants of Cas9 proteins include those that have been modified to reduce or eliminate nuclease activity. To enable sequence specific genomic regulation, nuclease-deactivated Cas9 (dCas9) has been engineered, and can be fused to a variety of effectors, such as transcriptional activators, repressors, and epigenetic modifiers (see, for example, Wang, H. et al. (2016) CRISPR/Cas9 in Genome Editing and Beyond, Annu. Rev. Biochem. 85: 227-264, and references cited therein). dCas9-fusion protein have been used to carry out other operations and for other applications (e.g. histone acetylation/methylation, deacetylation, DNA demethylation/methylation, transcription activation (e.g. VP64 activation domain), repression (e.g. KREB repressor domains), linking activation/repressor domains, various fluorophores for imaging, CRISPRa/CRISPRi, epigenetic editing, base editing (through attachment to DNA modifying domains), and attachment of other effectors and tags). In addition, inactive Cas9 and Cas13 (RNA-targeting enzyme described below), have been fused to base editing enzymes to directly perform gene editing in the absence of a homologous repair template (Gaudelli, N. M. et al. (2017) Programmable base editing of A•T to G•C in genomic DNA without DNA cleavage. Nature. doi:10.1038/nature24644) (Abudayyeh O. O. et al. (2017) RNA targeting with CRISPR-Cas13. Nature. doi:10.1038/nature24049).

Other Cas9 variants include Cas9 proteins that have been mutated in order to reduce off-target effects, such as those disclosed in WO 2016205613 A1 to THE BROAD INSTITUTE INC. et al. and in U.S. Pat. No. 9,512,446 B1 to The General Hospital Corporation. Other variants of Cas9 and its orthologues with improved specificity include eSPCas9 (Slaymaker I M, et al. (2016) Rationally engineered Cas9 nucleases with improved specificity. Science 351(6268):84-88) and SpCas9-HF (Kleinstiver B P, et al. (2016) High-fidelity CRISPR-Cas9 nucleases with no detectable genome-wide off-target effects. Nature 529(7587):490-495), and HypaCas9 (Chen, J. S. et al. (2017) Enhanced proofreading governs CRISPR-Cas9 targeting accuracy. Nature. 550: 407-410).

Still other Cas9 variants include variants in which only one nuclease domain is functional, resulting a nickase (nCas9) that is capable of introducing a single-stranded break (a “nick”) into the target sequence (Wang, H. et al. (2016) CRISPR/Cas9 in Genome Editing and Beyond, Annu. Rev. Biochem. 85: 227-264, and references cited therein).

A paired Cas9 nickase system involving fusion proteins (Guilinger J P, Thompson D B, & Liu D R (2014) Fusion of catalytically inactive Cas9 to FokI nuclease improves the specificity of genome modification. Nat Biotechnol 32(6):577-582) has also been used in order to reduce off-target effects.

In addition, Cas9-like synthetic proteins are known in the art (see, for example, U.S. Published Patent Application No. 2014-0315985, published Oct. 23, 2014) and are included in the scope of some embodiments of the present invention.

In some embodiments, the class 2 CRISPR-Cas protein is selected from a Cpfl protein, and variants thereof. Cpfl from Francisella novicida 1/112 has been characterized and found to have features distinct from Cas9 (Zetsche et al. (2015) Cpfl Is a Single RNA-Guided Endonuclease of a Class 2 CRISPR-Cas System, Zetsche et al. Cell 163, 759-71). Cpfl is a single RNA-guided endonuclease lacking tracrRNA, utilizes a T-rich protospacer-adjacent motif (PAM), and cleaves DNA via a staggered DNA double-stranded break. Sequence analysis has revealed that Cpf1 contains only a RuvC-like domain and lacks the HNH nuclease domain found in Cas9 (Zetsche et al. (2015) Cpfl Is a Single RNA-Guided Endonuclease of a Class 2 CRISPR-Cas System, Zetsche et al. Cell 163, 759-71). Variants of Cpf1 include, but are not limited to, Cpfl proteins that have been modified to reduce or eliminate nuclease activity (e.g. dCpf1), Cpfl proteins that have been mutated in order to reduce off-target effects, and synthetic or engineered/evolved versions of Cpfl proteins.

Above-noted WO2017/173054 to INTELLIA THERAPEUTICS, INC. discloses a number of CRISPR-Cas systems, including a variety of class 2 CRISPR-Cas proteins and variants thereof. Reference is also made to US 2017/0283831 A1 to The Broad Institute Inc. et al., which provides teaching around as well as an extensive list of articles and patent documents relating to delivery of a CRISPR-Cas protein complex and uses of an RNA guided endonuclease in cells and organisms. See also Shmakov, S. et al (2017) “Diversity and evolution of class 2 CRISPR-Cas systems” Nature Reviews Microbiology, Vol. 15, p. 169 and Supplementary Information.

Any of the class 2 CRISPR-Cas proteins disclosed in these or other references cited herein, or otherwise known to those of skill in the art, may be used in the methods described herein.

In some embodiments, the class 2 CRISPR-Cas protein is selected from Cas9, dCas9, nCas9, Cpfl, C2cl, C2c2, and C2c3 proteins, and variants or homologs thereof. In some embodiments, the class 2 CRISPR-Cas protein is Cas9. In yet some embodiments, the Cas9 protein is S. pyogenes Cas9. In still yet some embodiments, the Cas9 protein is an engineered variant of Cas9, such as eSpCas9, Cas9-HF1, or HypaCas9.

In some embodiments, the class 2 CRISPR-Cas protein is fused to an effector domain, thus forming a fusion protein; optionally, wherein the class 2 CRISPR-Cas protein lacks nuclease activity. In some embodiments, the fusion protein is functional (i.e. carries out an activity such as a function on target sequence, histones, etc.). In some embodiments, the fusion protein serves as a marker and may be non-functional. In yet some embodiments, the effector domain is a transcriptional activator, a repressor, a DNA methyltransferase, a histone methyl/acetyl transferase, a histone deacetylase, an enzyme capable of modifying DNA or RNA (e.g. base editors), or a fluorescent or tagging protein.

In some embodiments, the class 2 CRISPR-Cas protein has nuclease activity, and the method increases specificity of cleavage of the selected target nucleic acid sequence by the class 2 CRISPR-Cas protein.

In some embodiments, the selected target nucleic acid sequence is a DNA sequence. In some embodiments, the selected target nucleic acid sequence is a RNA sequence. It has recently been demonstrated that the class 2 type VI RNA-guided RNA-targeting CRISPR-Cas effector Cas13a (previously known as C2c2) can be engineered for mammalian cell RNA knockdown and binding (Abudayyeh O. O. et al. Nature. 2017 Oct. 12; 550(7675):280-284).

In some embodiments, the selected target nucleic acid sequence is immediately 5′ of a protospacer adjacent motif (PAM). Those of skill in the art will appreciate that the selected target nucleic acid sequence could be located in an alternate location for a specific class 2 CRISPR-Cas protein variant.

### Guide RNAs

In some embodiments, the guide RNA may comprise two RNA molecules—a first RNA molecule comprising a CRISPR-RNA (crRNA), and a second RNA molecule comprising a transactivating crRNA (tracrRNA). As known to those of skill in the art, the first and second RNA molecules may form a RNA duplex via the base pairing between the hairpin on the crRNA and the tracrRNA. As noted above, the crRNA contains an RNA sequence complementary to the selected target nucleic acid sequence. The tracrRNA acts as a bridge between the class 2 CRISPR-Cas protein (such as in the case of Cas 9). In other embodiments, the guide RNA may comprise a single RNA molecule and is known as a “single guide RNA” or “sgRNA”. In some embodiments, the sgRNA may comprise a crRNA covalently linked to a tracrRNA, such as via a linker. In some embodiments, the sgRNA is a Cas9 sgRNA capable of mediating RNA-guided nucleic acid binding and/or cleavage by a Cas9 protein. In some embodiments, the sgRNA is a Cpfl sgRNA capable of mediating RNA-guided nucleic acid binding and/or cleavage by a Cpfl protein. In certain embodiments, the guide RNA comprises a crRNA and tracrRNA sufficient for forming an active complex with a Cas9 protein and mediating RNA-guided nucleic acid binding and/or cleavage. In certain embodiments, the guide RNA comprises a crRNA sufficient for forming an active complex with a Cpfl protein and mediating RNA-guided nucleic acid binding and/or cleavage. In some embodiments, the guide RNA is used to direct RNA cleavage or editing by Cas13.

### Bridged Nucleic Acids

One embodiment of a modified nucleic acid comprises a bridged nucleic acid. As used herein, the term “bridged nucleic acid” will be understood to mean a nucleic acid having a structure wherein the degree of freedom of the nucleic acid is restricted through an intramolecular bond or crosslink.

First generation bridged nucleic acids, or locked nucleic acids (LNAs), comprise conformationally-restricted RNA nucleotides in which the 2′ oxygen in the ribose forms a covalent bond to the 4′ carbon, inducing N-type (C3′-endo) sugar puckering and preference for an A-form helix (You Y, Moreira B G, Behlke M A, & Owczarzy R (2006) Design of LNA probes that improve mismatch discrimination. Nucleic Acids Res 34(8):e60) (FIG. 1). LNAs display improved base stacking and thermal stability compared to RNA, resulting in highly efficient binding to complementary nucleic acids and improved mismatch discrimination (You Y, Moreira B G, Behlke M A, & Owczarzy R (2006) Design of LNA probes that improve mismatch discrimination. Nucleic Acids Res 34(8):e60; Vester B & Wengel J (2004) LNA (locked nucleic acid): high-affinity targeting of complementary RNA and DNA. Biochemistry 43(42):13233-13241), as well as nuclease resistance (Vester B & Wengel J (2004) LNA (locked nucleic acid): high-affinity targeting of complementary RNA and DNA. Biochemistry 43(42):13233-13241). They have been successfully used in numerous applications ranging from SNP detection assays (Vester B & Wengel J (2004) LNA (locked nucleic acid): high-affinity targeting of complementary RNA and DNA. Biochemistry 43(42):13233-13241) to siRNA (Elmen J, et al. (2005) Locked nucleic acid (LNA) mediated improvements in siRNA stability and functionality. Nucleic Acids Res 33(1):439-447). Recently, N-methyl substituted bridged nucleic acids (2′,4′BNA^{NC}[N-Me]) (FIG. 1) were designed to improve upon the original first generation LNA scaffold by introducing more conformational flexibility for DNA binding, even greater nuclease resistance due to steric bulk, and reduced cellular toxicity (Rahman S M, et al. (2008) Design, synthesis, and properties of 2′,4′-BNA(NC): a bridged nucleic acid analogue. J Am Chem Soc 130(14):4886-4896).

The incorporation of bridged nucleic acids at specific positions within a crRNA can be used to improve Cas9 DNA cleavage specificity by blocking the transition from the open conformation to the zipped conformation on off-target DNA sites. This finding demonstrates a new use for this recently developed synthetic nucleic acid technology, and it is expected that other current bridged nucleic acids (e.g. ENA (Varghese O P, et al. (2006) Conformationally constrained 2′-N,4′-C-ethylene-bridged thymidine (aza-ENA-T): synthesis, structure, physical, and biochemical studies of aza-ENA-T-modified oligonucleotides. J Am Chem Soc 128(47):15173-15187)) and future synthetic nucleic acids can be exploited to solve problems in enzyme specificity, with a goal to improve the specificity and safety of genome-editing agents for a wide-variety of experimental and clinical applications. In addition, other chemical modifications, not employing a 2′-4′ linkage may mimic the effects on the open/zipped conformation transition, making them analogous. Such other modifications include DNA or 2′OMe PAC.

A number of bridged nucleic acids are known to those of skill in the art and/or are available from commercial sources. In some embodiments, the bridged nucleic acid can be selected from those set forth in Table 1 below:

TABLE 1

| Name | Reference | Structure |
|---|---|---|
| 2′-O,3′-C- Methyleneuridine | Wengel, Jesper. ″Synthesis of 3′-C- and 4′-C-Branched Oligodeoxynucleotides and the Development of Locked Nucleic Acid (LNA).″ Acc. Chex. Res. 32 (1999): 301- 310. | 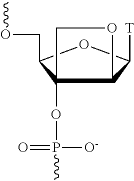 |
| 2′,4′-ENA 2′-O,4′-C- ethylene bridged nucleic acid | Mitsuoka, Yasunori, et al. ″A bridged nucleic acid, 2′,4′-BNA^{COC}: synthesis of fully modified oligonucleo- tides bearing thymine, 5- methylcytosine, adenine and guanine 2′,4′-BNA^{COC} monomers and RNA- | 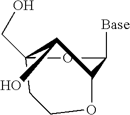 |
| | selective nucleic-acid | |
| | recognition.″ Nucleic Acids | |
| | Research 37. (2009): 1225- | |
| | 1238. | |
| 2′-O,3′-C- Methylenecytidine | Obika, Satoshi, et al. ″Synthesis of 2′-O,4′-C- Methyleneuridine and -cytidine. Novel Bicyclic Nucleosides Having a Fixed C₃,-endo Sugar Puckering.″ Tetrahedron Letters 38 (1997): 8735- | 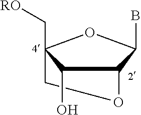 |
| | 8738. | |
| 2′-O,4′-C- Methyleneuridine | Obika, Satoshi, et al. ″Synthesis of 2′-O,4′-C- Methyleneuridine and -cytidine Novel Bicyclic Nucleosides Having a FixedC₃,-endo Sugar Puckering.″ Tetrahedron Letters 38 (1997): 8735- | 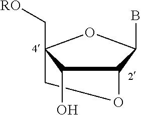 |
| | 8738. | |
| 2′,4′-BNA-1- isoquinolone | Hari, Yoshiyuki, et al. ″Selective recognition of CG interruption by 2′,4′- BNA having 1-isoquinolone as a nucleobase in a pyrimidine motif triplex formation.″ Tetrahedron 59. (2003): 5123-5128. | 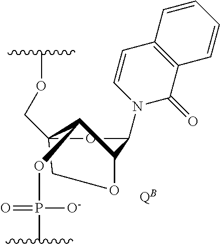 [SMILES: *OC[C@@]12CO[C@H]([C@H](n3ccc4ccccc4c3=O)O1)[C@@H]2OP(*)(=O)[O-]] |
| 2′,4′-BNA-2- pyridone | Obika, Satoshi, et al. ″A 2′,4′-Bridged Nucleic Acid Containing 2-Pyridone as a Nucleobase: Efficieny Recognition of a C.G Interruption by Triplex Formation with a Pyrimidine Motif.″ Angew. Chem. Int. Ed. 40. (2001): 2079-2081. | 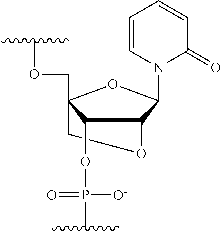 [SMILES: *OC[C@@]12COC([C@H](n3ccccc3=O)O1)[C@H]2OP(*)(=O)[O-]] |
| 2′,4′-BNA-TeNA | Mitsuoka, Yasunori, et al. ″Triazole- and Tetrazole- Bridged Nucleic Acids: Synthesis, Duplex Stability, Nuclease Resistance, and in vitro and in vivo Antisense Potency.″ J. Org. Chem. 82. (2016): 12-24. | 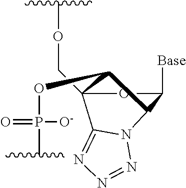 |
| 2′,4′-BNA-TrNA | Mitsuoka, Yasunori, et al. ″Triazole- and Tetrazole- Bridged Nucleic Acids: Synthesis, Duplex Stability, Nuclease Resistance, and in vitro and in vivo Antisense Potency.″ J. Org. Chem. 82. (2016): 12-24. | 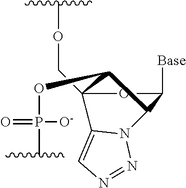 |
| 2′,4′-BNA^{COC} | Mitsuoka, Yasunori, et al. ″A bridged nucleic acid, 2′,4′-BNA^{COC}: synthesis of fully modified oligonucleo- tides bearing thymine, 5- methylcytosine, adenine and guanine 2′,4′-BNA^{COC} monomers and RNA- | 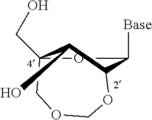 |
| | selective nucleic-acid | |
| | recognition.″ Nucleic Acids | |
| | Research 37. (2009): 1225- | |
| | 1238. | |
| 2′,4′-BNA^{NC} [NBn] | Rahman, S.M. Abdur, et al. ″Design, Synthesis, and Properties of 2′,4′-BNA^{NC}: A Bridged Nucleic Acid Analogue.″ J. Am. Chem. Soc. 14. (2008): 4886-4896. | 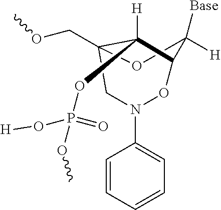 |
| 2′,4′-BNA^{NC} [NH] | Rahman, S.M. Abdur, et al. ″Design, Synthesis, and Properties of 2′,4′-BNA^{NC}: A Bridged Nucleic Acid Analogue.″ J. Am. Chem. Soc. 14. (2008): 4886-4896. | 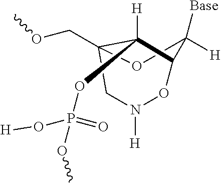 |
| 2′,4′-BNA^{NC} [NMe] | Rahman, S.M. Abdur, et al. ″Design, Synthesis, and Properties of 2′,4′-BNA^{NC}: A Bridged Nucleic Acid Analogue.″ J. Am. Chem. Soc. 14. (2008): 4886-4896. | 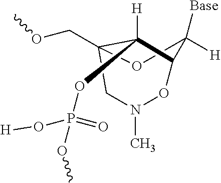 |
| 3′-amino-2′,4′- BNA | Obika, Satoshi, et al. ″Synthesis and properties of 3′-amino-2′,4′-BNA, a bridged nucleic acid with a N3′→P5′ phosphoramidate linkage.″ Bioorg. Med. Chem. 16 (2008): 9230- 9237. | 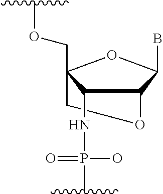 |
| 3′-O,4′-C- methyleneuridine | Obika, Satoshi, et al. ″Preparation and properties of 2′,5′-linked oligonucleotide analogues containing 3′-O,4′-C- methyleneribonucleosides.″ Bioorg. Med. Chem. Lett. 4. (1999): 515-518. | 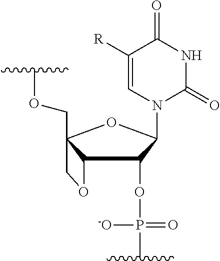 |
| AmNA | Mitsuoka, Yasunori, et al. ″Sulfonamide-Bridged Nucleic Acid: Synthesis, High RNA Selective Hybridization, and High Nuclease Resistance.″ Org. Lett. 21. (2014): 5640-5643. | 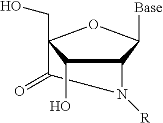 |
| DpNA (3,4-dihydro-2H- pyran bridge moiety) | Shrestha, Ajaya R, et al. ″Synthesis and Properties of a Bridged Nucleic Acid with a Perhydro-1,2-oxazin- 3-one Ring.″ J. Org. Chem. 24. (2011): 9891-9899. | 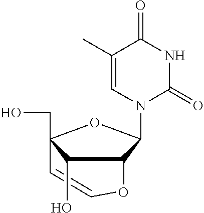 [SMILES: Cc1cn([C@@H]2O[C@@]3(CO)C=COC2[C@H]3O)c(=O)[nH]c1=O] |
| EoNA | Hari, Yoshiyuki, et al. ″Synthesis and Properties of 2′,-O,4′-C-Ethyleneoxy Bridged 5-Methyl- uridine.″ Org. Lett. 14. (2013): 3702-3705. | 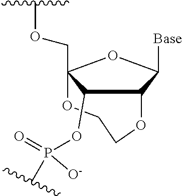 |
| GuNA (Guanidine Bridged Nucleic Acid) | Shrestha, Ajaya R, et al. ″Guanidine bridged nucleic acid (GuNA): an effect of cationic bridged nucleic acid on DNA binding affinity.″ Chem. Commun. 50. (2014): 575-577. | 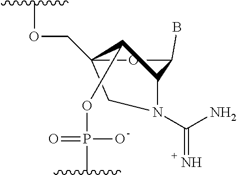 |
| HxNA | Shrestha, Ajaya R, et al. ″Synthesis and Properties of a Bridged Nucleic Acid with a Perhydro-1,2-oxazin- 3-one Ring.″ J. Org. Chem. 24. (2011): 9891-9899. | 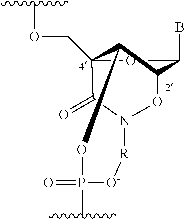 |
| PrNA | Mitsuoka, Yasunori, et al. ″A bridged nucleic acid, 2′,4′-BNA^{COC}: synthesis of fully modified oligonucleo- tides bearing thymine, 5- methylcytosine, adenine and guanine 2’,4’-BNA^{COC} monomers and RNA- | 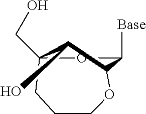 |
| | selective nucleic-acid | |
| | recognition.″ Nucleic Acids | |
| | Research 37. (2009): 1225- | |
| | 1238. | |
| scpBNA (2′-O,4′-C-spiro- cycloprepylene bridged nucleic acid) | Yamaguchi, Takao, et al. ″Synthesis and properties of 2′-O,4′-C-spirocyclo- propylene bridged nucleic acid (scpBNA), an analogue of 2′,4′-BNA/LNA bearing a cyclopropane ring.″ Chem. | 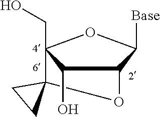 |
| | Commun. 51. (2015): 9737- | |
| | 9740. | |
| Six-membered AmNA | Mitsuoka, Yasunori, et al. ″Sulfonamide-Bridged Nucleic Acid: Synthesis, High RNA Selective Hybridization, and High Nuclease Resistance.″ Org. Lett. 21. (2014): 5640- 5643. | 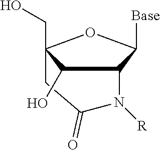 |
| SuNA | Mitsuoka, Yasunori, et al. ″Sulfonamide-Bridged Nucleic Acid: Synthesis, High RNA Selective Hybridization, and High Nuclease Resistance.″ Org. Lett. 21. (2014): 5640-5643. | 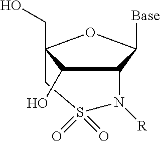 |
| Urea-BNA | Mitsuoka, Yasunori, et al. ″Sulfonamide-Bridged Nucleic Acid: Synthesis, High RNA Selective Hybridization, and High Nuclease Resistance.″ Org. Lett. 21. (2014): 5640-5643. | 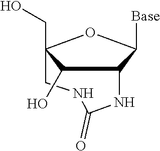 |
| α-L-LNA | Sørensen, Mads D, et al. ″α-L-ribo-Configured Locked Nucleic Acid (α- L-LNA): Synthesis and Properties.″ J. Am. Chem. Soc. 124. (2002): 2164-2176. | 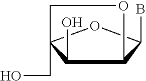 |
| bicyclo-DNA | Bolli, M, et al. ″Bicylco- DNA: a Hoogsteen-selective pairing system.″ Chem. Biol. 3. (1996): 197-206. | 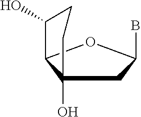 |
| 5-methyl- 2′-O,4′-C- methyleneuridine | Obika, Satoshi, et al. ″Synthesis and properties of 3′-amino-2′,4′-BNA, a bridged nucleic acid with a N3′→P5′ phosphoramidate linkage″. Bioorg. Med. Chem. 16. (2008): 9230- 9237. | 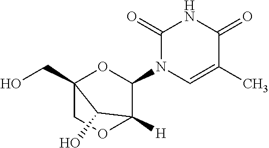 [SMILES: Cc1cn([C@@H]2O[C@@]3(CO)CO[C@@H]2[C@@H]3O)c(=O)[nH]c1=O] |
| 5-bromo- 2′-O,4′-C- methyleneuridine | Obika, Satoshi, et al. ″Synthesis and properties of 3′-amino-2′,4′-BNA, a bridged nucleic acid with a N3′→P5′ phosphoramidate linkage″. Bioorg. Med. Chem. 16. (2008): 9230- 9237. | 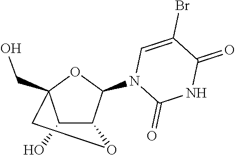 [SMILES: O=c1[nH]c(=O)n([C@@H]2O[C@@]3(CO)CO[C@@H]2[C@@H]3O)cc1Br] |
| 3′-O-benzyl-5′- O-mesyl-5-methyl- 2′-O,4′-C- methyleneuridine | Obika, Satoshi, et al. ″Synthesis and properties of 3′-amino-2′,4′-BNA, a bridged nucleic acid with a N3′→P5′ phosphoramidate linkage″. Bioorg. Med. Chem. 16. (2008): 9230- 9237. | 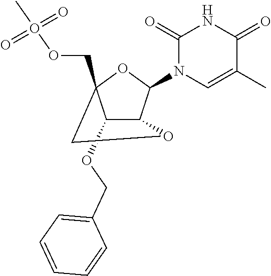 |
| Benzylidene acetal-type bridged nucleic acids (BA-BNAs) | Kodama, T. ″Synthesis and Characterization of Benzylidene Acetal-Type Bridged Nucleic Acids (BA-BNA).″ Curr. Protoc. Nucleic Acid Chem. 58. (2014): 1-22. | 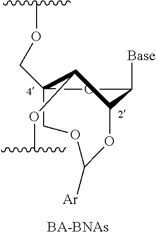 |

In some embodiments, the bridged nucleic acid may comprise

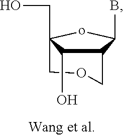

or

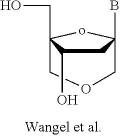

or another bridged nucleic acid described in Takeshi Imanishi and Satoshi Obika, “BNAs: novel nucleic acid analogs with a bridged sugar moiety” Chemical Communications 2002 (Issue 16).

In some embodiments, the bridged nucleic acid is a 2′,4′-bridged nucleic acid—i.e. the bridged nucleic acid comprises a bridge incorporated at the 2′-, 4′-position of the sugar ring. A number of the bridged nucleic acids appearing in Table 1 above include a bridge incorporated at the 2′-, 4′-position of the sugar ring.

As noted above, bridged nucleic acids can be obtained from commercial sources. For example, BioSynthesis Inc. is a commercial source of bridged nucleic acids and has published a number of known 2′,4′-bridged nucleic acids, as shown below:

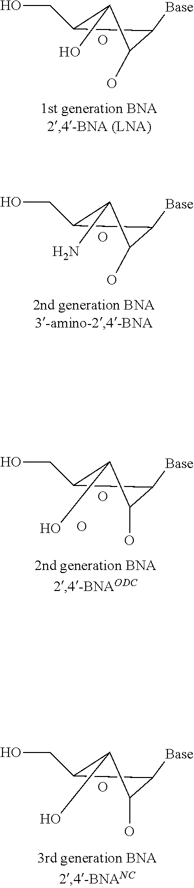

U.S. Pat. No. 6,770,748 B2 to Takeshi Imanishi also discloses bridged nucleic acids that can be incorporated into an oligonucleotide or polynucleotide, having the general formula:

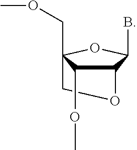

U.S. Pat. No. 6,770,748 B2 to Takeshi Imanishi defines B as being a pyrimidine or purine nucleic acid base, or an analogue thereof.

Other bridged nucleic acids are taught in U.S. Pat. Nos. 8,153,365, 8,080,644, 7,060,809, 7,084,125, 7,060,809, 7,053,207, 6,670,461, 6,436,640, 6,316,198 to Exiqon A/S.

Still other bridged nucleic acids that can be incorporated into an oligonucleotide or polynucleotide are taught in U.S. Pat. No. 7,427,672 B2 to Takeshi Imanishi, having the general formula:

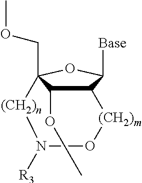

wherein, according to embodiments defined in U.S. Pat. No. 7,427,672 B2 to Takeshi Imanishi, R₃ represents a hydrogen atom, an alkyl group (such as straight chain or branched chain alkyl group having 1 to 20 carbon atoms), an alkenyl group (such as straight chain or branched chain alkenyl group having 2 to 20 carbon atoms), a cycloalkyl group (such as a cycloalkyl group having 3 to 10 carbon atoms), an aryl group (such as a monovalent substituent having 6 to 14 carbon atoms which remains after removing one hydrogen atom from an aromatic hydrocarbon group, e.g. phenyl), an aralkyl group (such as an alkyl group having 1 to 6 carbon atoms which has been substituted by an aryl group), an acyl group (such as alkylcarbonyl groups), a sulfonyl group (e.g. alkyl or aryl substituted), and m denotes an integer of 0 to 2, and n denotes an integer of 1 to 3.

In some embodiments, the at least one bridged nucleic acid used in the methods, guide RNA, kits, and complexes described herein is independently selected from any of the bridged nucleic acids described or referred to herein.

In some embodiments, the bridged nucleic acid is independently selected from:

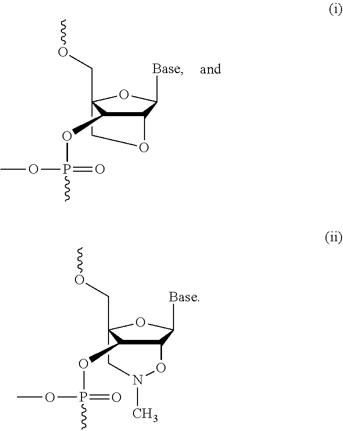

It will be understood to those of skill in the art that the base in the bridged nucleic acid is a pyrimidine or purine nucleic acid base, and can be thymine, uracil, cytosine, adenine, guanine, or derivatives/analogues thereof. Analogues of pyrimidine or purine nucleic acids are known to those of skill in the art, such as those outlined in references cited herein, for example U.S. Pat. No. 6,770,748 B2 or U.S. Pat. No. 7,427,672 B2 to Takeshi Imanishi, and references cited therein.

In some embodiments, the complementarity region at the 5′ end of the guide RNA comprises from about 16 to about 22 nucleotides. In some embodiments, the complementarity region at the 5′ end of the guide RNA comprises about 20 nucleotides. In yet some embodiments, the guide RNA comprises 3 or 4 bridged nucleic acids located between positions 4 and 17 or 15 and 20 from the 5′ end of the guide RNA. In still yet some embodiments, the bridged nucleic acids are positioned adjacent to one another. In some embodiments, the bridged nucleic acids are positioned in alternating positions relative to one another (spaced apart by a single unmodified nucleic acid). In yet some embodiments, the bridged nucleic acids are located between positions 9 and 14 from the 5′ end of the guide RNA.

In some embodiments, the target nucleic acid molecule comprises an off-target nucleic acid sequence, wherein the off-target nucleic acid sequence comprises at least one nucleotide that differs from the selected target nucleic acid sequence at a mismatch position, wherein the at least one modified nucleic acid is located on the guide RNA at a position corresponding, adjacent or proximal to the mismatch position.

In some embodiments, the guide RNA comprises (i) a crRNA or a tracrRNA, or (ii) a crRNA and a tracrRNA, or (iii) a single guide RNA.

In some embodiments of the methods described herein, the step of contacting the nucleic acid molecule comprising the selected target nucleic acid sequence with the complex comprising the class 2 CRISPR-Cas protein and the guide RNA occurs in vitro. In some embodiments, the step of contacting the nucleic acid molecule comprising the selected target nucleic acid sequence with the complex comprising the class 2 CRISPR-Cas protein and the guide RNA occurs in vivo.

In some embodiments, a guide RNA is provided, wherein the guide RNA comprises a complementarity region at the 5′ end of the guide RNA that binds to a complementary strand of a selected target nucleic acid sequence within a nucleic acid molecule, wherein the guide RNA comprises at least one modified nucleic acid within the complementarity region. In some embodiments, the selected target nucleic acid sequence is immediately 5′ of a protospacer adjacent motif (PAM). In some embodiments, the at least one modified nucleic acid is a bridged nucleic acid, and is independently selected from any of the bridged nucleic acids outlined above or a nucleic acid analogue that results in a similar conformational transition state with the enzyme (e.g. similar effects on specificity observed with DNA or 2′OMe PAC). In some embodiments, the at least one bridged nucleic acid independently selected from a 2′,4′-bridged nucleic acid. In still yet some embodiments, the at least one bridged nucleic acid is independently selected from:

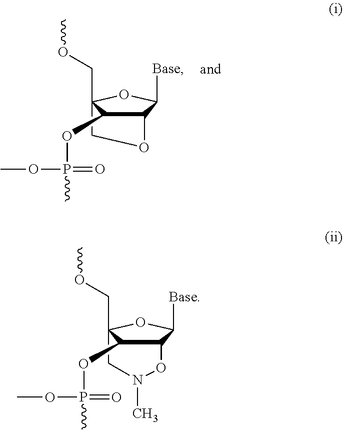

### Other Modified Nucleic Acids

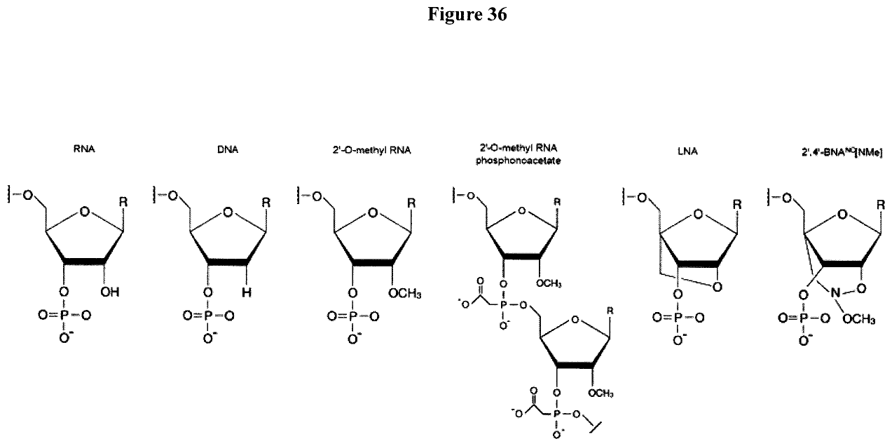

As well as the bridged nucleic acids described above, in some embodiments, the modified nucleic acid may comprise a modified nucleic acid shown in FIG. 36, or a conformationally similar variant thereof. The modified nucleic acid may comprise a deoxyribonucleic acid (DNA), 2′-O-methyl RNA, or a 2′-O-methyl RNA phosphonoacetate.

In some embodiments, the guide RNA binds to a complementary strand of a selected target nucleic acid sequence within a nucleic acid molecule, wherein the nucleic acid molecule is DNA. In some embodiments, the nucleic acid molecule is RNA. In some embodiments, the complementarity region at the 5′ end of the guide RNA comprises from about 16 to about 22 nucleotides. In some embodiments, the complementarity region comprises about 20 nucleotides. In some embodiments, the guide RNA comprises 3 or 4 modified nucleic acids located between positions 4 and 17 or positions 15 to 20 (inclusive) from the 5′ end of the guide RNA. In some embodiments, the modified nucleic acids are positioned adjacent to one another. In some other embodiments, the modified nucleic acids are positioned in alternating positions relative to one another (separated by a single unmodified nucleic acid). In some embodiments, the modified nucleic acids are located between positions 9 and 14 from the 5′ end of the guide RNA.

In some embodiments, the nucleic acid molecule comprises an off-target nucleic acid sequence, wherein the off-target nucleic acid sequence comprises at least one nucleotide that differs from the selected target nucleic acid sequence at a mismatch position, wherein the at least one modified nucleic acid is located on the guide RNA at a position corresponding, adjacent or proximal to the mismatch position. In some embodiments, the guide RNA comprises (i) a crRNA and/or a tracrRNA, or (ii) a single guide RNA.

In some embodiments, the guide RNA retains the ability to form a complex with a class 2 CRISPR-Cas protein, which may include any such protein described or referred to herein. In some embodiments, the class 2 CRISPR-Cas protein is selected from Cas9, Cpfl, C2cl, C2c2, and C2c3 proteins, and variants or homologs thereof. In some embodiments, the class 2 CRISPR-Cas protein is Cas9. In yet some embodiments, the Cas9 protein is S. pyogenes Cas9. In some embodiments, the Cas9 protein is an engineered variant of Cas9, such as eSpCas9, Cas9-HF1, or HypaCas9. In some embodiments, the class 2 CRISPR-Cas protein is fused to an effector domain, thus forming a fusion protein; optionally, wherein the class 2 CRISPR-Cas protein lacks nuclease activity. In some embodiments, the fusion protein is functional (i.e. carries out an activity such as a function on target sequence, histones, etc.). In some embodiments, the fusion protein is non-functional and serves as a marker. In yet some embodiments, the effector domain is a transcriptional activator, a repressor, a DNA methyltransferase, a histone methyl/acetyl transferase, a histone deacetylase, an enzyme capable of modifying DNA or RNA (e.g. base editors), or a fluorescent or tagging protein. In some embodiments, the class 2 CRISPR-Cas protein has nuclease activity.

In some embodiments, there is provided a kit comprising a guide RNA as described herein and a class 2 CRISPR-Cas protein as described herein. The kit may optionally include instructions for use.

In some embodiments, there is provided a complex comprising: a class 2 CRISPR-Cas protein; and a guide RNA comprising a complementarity region at the 5′ end of the guide RNA that binds to a complementary strand of a selected target nucleic acid sequence within a nucleic acid molecule, wherein the guide RNA comprises at least one bridged nucleic acid within the complementarity region. In some embodiments, the selected target nucleic acid sequence is immediately 5′ of a protospacer adjacent motif (PAM). In some embodiments, the nucleic acid molecule is DNA. In yet some embodiments, the nucleic acid molecule is RNA. In yet some embodiments, the nucleic acid molecule comprises an off-target nucleic acid sequence, wherein the off-target nucleic acid sequence comprises at least one nucleotide that differs from the selected target nucleic acid sequence at a mismatch position, wherein the at least one bridged nucleic acid is located on the guide RNA at a position corresponding, adjacent or proximal to the mismatch position. The class 2 CRISPR-Cas protein in such complex can be any of the class 2 CRISPR-Cas proteins as defined above. Likewise, the guide RNA in such complex can be any of the guide RNAs as defined above, incorporating any of the modified nucleic acids as defined above.

As noted above, the step of contacting the nucleic acid molecule comprising the selected target nucleic acid sequence with the complex comprising the class 2 CRISPR-Cas protein and the guide RNA can occur in vitro or in vivo. A number of delivery methods are known to the skilled worker for use in modified nucleic acid-modified guide RNA applications, both for cellular and in vivo applications. These include, but are not limited to, the following:

A. Cationic Lipid-Based Vectors:

Lipofectamine RNAiMAX, Lipofectamine 2000 (LF2K), Lipofectamine 3000 (LF3K), Lipofectamine MessengerMAX, TurboFect, and Xfect (see, for example, Zuris, J., Thompson, D., Shu, Y., Guilinger, J., Bessen, J., Hu, J., Maeder, M., Joung, J., Chen, Z. and Liu, D. (2014). Cationic lipid-mediated delivery of proteins enables efficient protein-based genome editing in vitro and in vivo. Nature Biotechnology, 33(1), pp. 73-80).

Lipofectamine CRISPRMAX (see, for example, Yu, X., Liang, X., Xie, H., Kumar, S., Ravinder, N., Potter, J., de Mollerat du Jeu, X. and Chesnut, J. (2016). Improved delivery of Cas9 protein/gRNA complexes using lipofectamine CRISPRMAX. Biotechnology Letters, 38(6), pp. 919-929).

B. Cationic Polymer-Based Vectors:

Polyethyleneimine (PEI) (see, for example, Wightman L., Kircheis R., Rössler V., Carotta S., Ruzicka R., Kursa M., Wagner E. Different behavior of branched and linear polyethylenimine for gene delivery in vitro and in vivo. J. Gene Med. 2011; 3:362-372. doi: 10.1002/jgm.187; and Sun, W. et al. Self-assembled DNA nanoclews for the efficient delivery of CRISPR-Cas9 for genome editing. Angew. Chem. Int. Ed. 54, 12029-12033 (2015)).

C. Electroporation:

Lonza Nucleofector—(see, for example, Liu, J., Gaj, T., Yang, Y., Wang, N., Shui, S., Kim, S., Kanchiswamy, C., Kim, J. and Barbas, C. (2015). Efficient delivery of nuclease proteins for genome editing in human stem cells and primary cells. Nature Protocols, 10(11), pp. 1842-1⁸⁵⁹.

Neon Electroporation System (see, for example, Liang, X., Potter, J., Kumar, S., Zou, Y., Quintanilla, R., Sridharan, M., Carte, J., Chen, W., Roark, N., Ranganathan, S., Ravinder, N. and Chesnut, J. (2015). Rapid and highly efficient mammalian cell engineering via Cas9 protein transfection. Journal of Biotechnology, 208, pp. 44-53).

D. Cell Penetrating Peptide (CPP):

GFP-tagged Cas9 (see, for example, Zuris, J., Thompson, D., Shu, Y., Guilinger, J., Bessen, J., Hu, J., Maeder, M., Joung, J., Chen, Z. and Liu, D. (2014). Cationic lipid-mediated delivery of proteins enables efficient protein-based genome editing in vitro and in vivo. Nature Biotechnology, 33(1), pp. 73-80).

CPP-conjugated Cas9 and gRNA (see, for example, Ramakrishna, S., Kwaku Dad, A., Beloor, J., Gopalappa, R., Lee, S. and Kim, H. (2014). Gene disruption by cell-penetrating peptide-mediated delivery of Cas9 protein and guide RNA. Genome Research, 24(6), pp. 1020-1027).

Co-incubation of Cas9:gRNA and tetramethylrhodamine TAT dimer (dfTAT) (see, for example, Erazo-Oliveras, A., Najjar, K., Dayani, L., Wang, T., Johnson, G. and Pellois, J. (2014). Protein delivery into live cells by incubation with an endosomolytic agent. Nature Methods, 11(8), pp. 861-867).

E. Nanoparticles:

Gold nanoparticles (see, for example, Mout, R., Ray, M., Yesilbag Tonga, G., Lee, Y., Tay, T., Sasaki, K. and Rotello, V. (2017). Direct Cytosolic Delivery of CRISPR/Cas9-Ribonucleoprotein for Efficient Gene Editing. ACS Nano, 11(3), pp. 2452-2458).

7C1 nanoparticles (see, for example, Platt R J, Chen S, Zhou Y, Yim M J, Swiech L, Kempton H R, Dahlman J E, Parnas O, Eisenhaure T M, Jovanovic M, Graham D B, Jhunjhunwala S, Heidenreich M, Xavier R J, Langer R, Anderson D G, Hacohen N, Regev A, Feng G, Sharp P A, Zhang F. Cell. 2014 Oct. 9; 159(2):440-55).

F. Microinjection:

Single-cell embryonic microinjection (see, for example, Shao, Y., Guan, Y., Wang, L., Qiu, Z., Liu, M., Chen, Y., Wu, L., Li, Y., Ma, X., Liu, M. and Li, D. (2014). CRISPR/Cas-mediated genome editing in the rat via direct injection of one-cell embryos. Nature Protocols, 9(10), pp. 2493-2512).

Hydrodynamic injection (see, for example, Yin, H. et al. Genome editing with Cas9 in adult mice corrects a disease mutation and phenotype. Nat. Biotechnol. 32, 551-553 (2014)).

G. Mechanical Deformation:

Microfluidic devices (see, for example, Han, X. et al. CRISPR-Cas9 delivery to hard-to-transfect cells via membrane deformation. Sci. Adv. 1, e1500454 (2015)).

H. iTOP:

Combination hypertonicity-induced micropinocytosis and treatment with the transduction compound propanebetaine (see, for example, D'Astolfo, D. S. et al. Efficient intracellular delivery of native proteins. Cell 161, 674-690 (2015)).

I. Incorporation into Viral Particles:

Artificial incorporation into viral particles during assembly, or via electroporation or chemical manipulation. Once inside, viral particles expressing both Cas9 and the modified gRNA can be delivered to cells to perform genome editing.

As well, use and delivery of Cas9 is described in detail in Wang, H. et al. (2016) CRISPR/Cas9 in Genome Editing and Beyond, Annu. Rev. Biochem. 85: 227-264, and references cited therein.

### Applications

The methods of increasing specificity of binding of a class 2 CRISPR-Cas protein-guide RNA complex to a selected target nucleic acid sequence by incorporating at least one modified nucleic acid within the complementarity region of the guide RNA, as well as the class 2 CRISPR-Cas protein-guide RNA complexes, guide RNAs, and kits described herein, can be used in a wide variety of applications and can be used as a tool to study to study Cas based system dynamics, mechanism, or structure. The methods, complexes, guide RNAs, and kits described herein can be used to improve the editing of all genomes, including but not limited to mammalian, plant, bacterial, archaea, etc., for cleavage of DNA in vitro, or cleavage of DNA in cells either for the purpose of gene knockout or gene knock-in via homologous recombination, non-homologous end-joining (NHEJ) or other mechanisms. Other uses include as a therapeutic to treat human embryonic cells with improved specificity though gene editing, as a therapeutic to treat human somatic cell disorders through delivery into multiple cells, uses in agriculture for the specific engineering of livestock, plants, etc., uses in ecological engineering or for use in gene drive technology, for the modification of cancer cell lines (e.g. HEK293, U2OS, K562), model organisms (mice, rats, flies, nematodes, plants, salamanders, frogs, monkeys, humans), biotechnology applications (rice, wheat, tobacco, sorghum), modification of bacteria/viruses/fungi, pathogenic or non-pathogenic, use in organoids, human embryonic stem cells (hESC), induced pluripotent stem cells (iPSCs). Various applications of Cas9 systems are outlined in Wang, H. et al. (2016) CRISPR/Cas9 in Genome Editing and Beyond, Annu. Rev. Biochem. 85: 227-264, and references cited therein, and it is expected that the methods, guide RNAs, complexes, and kits described herein would be useful in any of these applications.

The methods of increasing specificity may also apply to applications involving inactive (dCas9), nickase (nCas9) or otherwise modified variants of Cas9 fused to effector domains for modulating transcription, performing base editing, or other functions previously described (see Wang, H. et al. (2016) CRISPR/Cas9 in Genome Editing and Beyond, Annu. Rev. Biochem. 85: 227-264, and references cited therein).

### Examples

To gain a better understanding of the invention described herein, the following examples are set forth. It should be understood that these examples are for illustrative purposes only. Therefore, they should not limit the scope of this invention in any way.

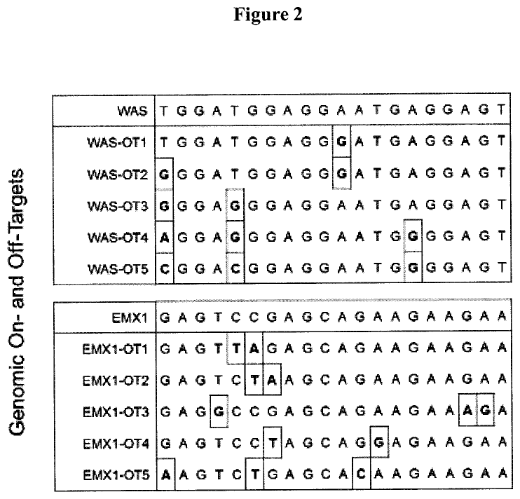

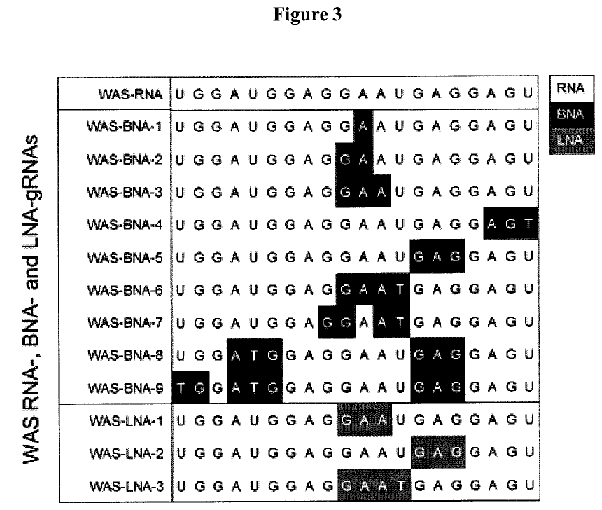

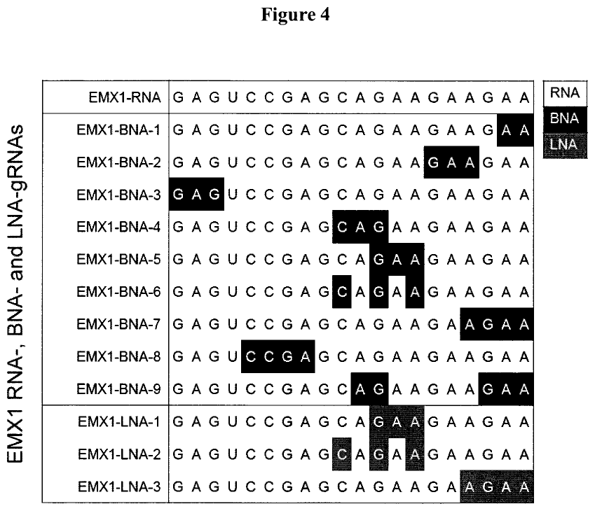

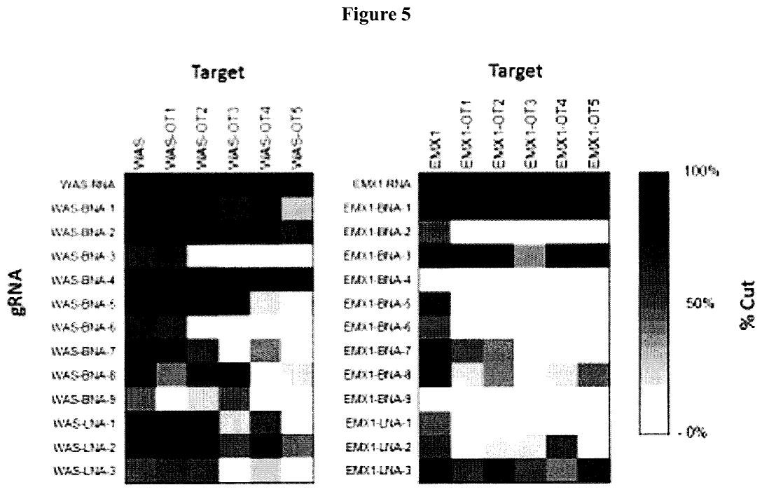

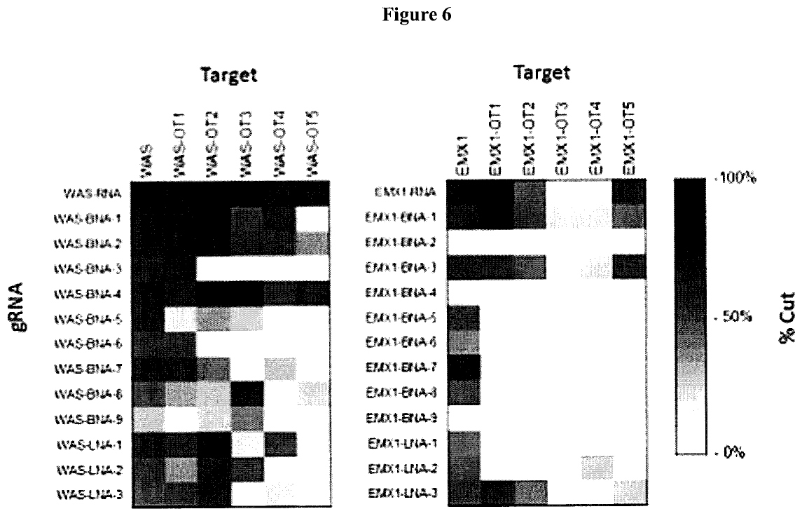

To test if incorporation of BNA^{NC}[N-Me] in crRNAs could be used to improve Cas9 cleavage specificity, we selected two previously characterized crRNAs directed towards the WAS and EMX1 genes, for which in vitro and cellular off-target sites have been identified (FIG. 2—off-target sites), and designed variants with BNA^{NC} substitutions (i.e. 2′,4′-BNA^{NC}[N-Me] as shown in FIG. 1; referred to herein as “BNA^{NC}” and also abbreviated as “BNA” in the Figures and description, for convenience) and some with LNA substitutions (i.e. 2′,4′-BNA in FIG. 1; referred to herein and in the Figures as “LNA”). See FIG. 3 and FIG. 4. Previous work has demonstrated that local mismatch discrimination can be improved in RNA-DNA hybrids when LNAs are incorporated in the vicinity of mismatches, with an LNA triplet centered on the mismatch yielding the best results (You Y, Moreira B G, Behlke M A, & Owczarzy R (2006) Design of LNA probes that improve mismatch discrimination. Nucleic Acids Res 34(8):e60). Therefore, we generated a series of 8 crRNAs with substitutions of 1, 2, 3 or 4 BNA^{NC}s (or in pairs) corresponding to the key mismatch positions of the five most abundant cellular off-target sites of the WAS and EMX1 crRNAs respectively as well first-generation LNA versions of three of these for comparison (FIG. 3 and FIG. 4). Using an in vitro cleavage assay, we screened the ability of Cas9 to cleave these previously described on- and off-target sequences (Wang X, et al. (2014) Unbiased detection of off-target cleavage by CRISPR-Cas9 and TALENs using integrase defective lentiviral vectors. Nat. Biotechnol. 33(2):175-178; Kim D, et al. (2016) Genome-wide target specificities of CRISPR-Cas9 nucleases revealed by multiplex Digenome-seq. Genome Res. 26:405-415) using the original crRNAs and these BNA^{NC-} and LNA-substituted crRNAs (BNA-1-8, LNA1-3) at two different doses. For the WAS gene set, we found that substitution of the centrally located GAA triplet in positions 9-11 of the crRNA with BNA^{NC} (WAS-BNA-3) greatly diminished off-target cleavage on all but 1 off-target sequences. Interestingly, the off-target sequence that was unaffected contained a single A-G mismatch located within the substituted triplet. crRNAs containing substitutions of only 1 or 2 BNA^{NC}s, and substitutions in flanking positions within the crRNA demonstrated more heterogeneous effects on specificity. crRNAs containing LNA triplets mirrored the effects of BNA^{NC}s, but improved specificity to a lesser extent (FIG. 5 and FIG. 6) A similar trend was observed for the EMX1 gene set, at both low and high doses of RNP. Remarkably, BNA^{NC} substitutions at positions 12-14 of the EMX1 crRNA (EMX-BNA-5) abolished cleavage on all five off-target sites tested, while only marginally decreasing on-target cleavage activity. In this case, EMX1-LNA-1 also abolished all off-target cleavage activity, and behaved similarly to EMX-BNA-6, its corresponding counterpart (FIG. 5 and FIG. 6).

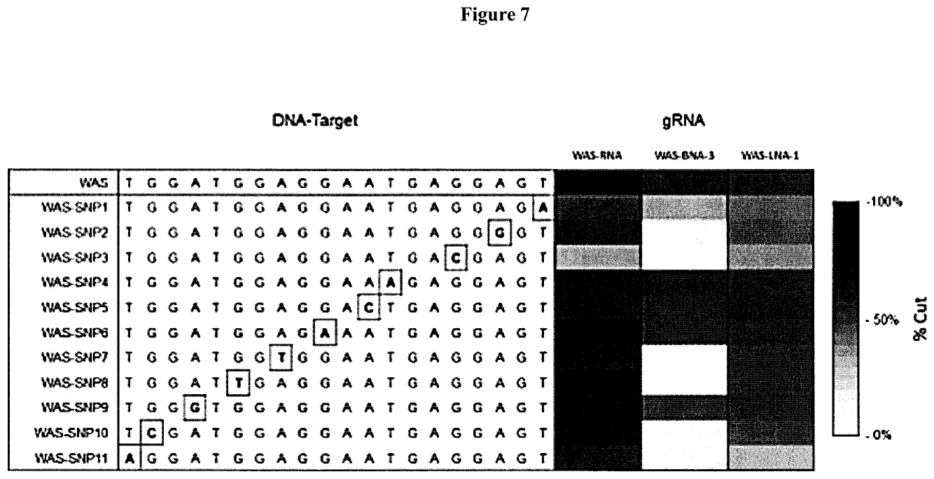

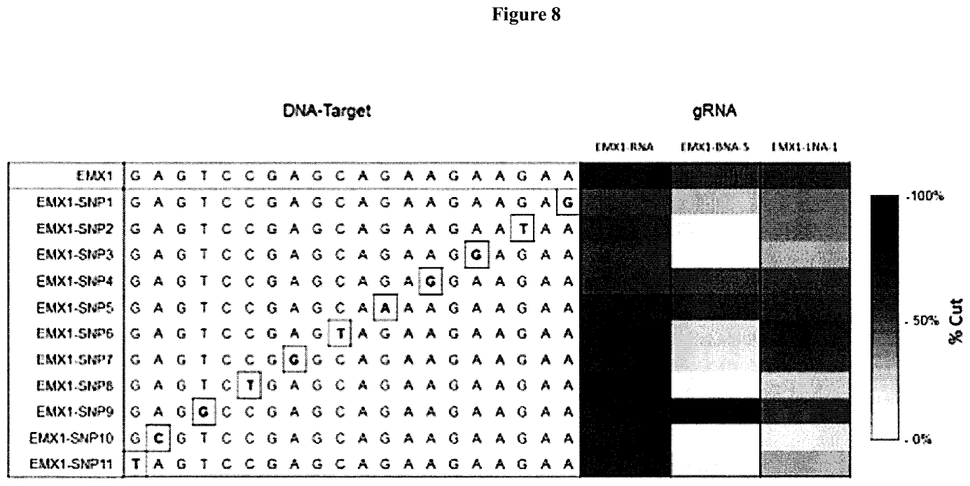

The Cas9 system is generally not effective in resolving single nucleotide polymorphisms (SNPs) targeted by the PAM-distal portion of guide sequence (Jiang W, Bikard D, Cox D, Zhang F, & Marraffini L A (2013) RNA-guided editing of bacterial genomes using CRISPR-Cas systems. Nat Biotechnol 31(3):233-239). Based on our finding that BNA^{NC}-substituted crRNAs can improve specificity, we speculated that they might improve Cas9 SNP discrimination. To test this hypothesis, we generated a series of target sequences corresponding to the WAS and EMX1 sites bearing individual mutations at 2 bp intervals (see Appendix 1) and assayed their ability to be cleaved in vitro by Cas9 using either the unmodified crRNA, or the most-specific BNA^{NC}-substituted crRNA. For the WAS target, we found that WAS-BNA-3 dramatically improved SNP discrimination up to 10-fold at both PAM-proximal and PAM-distal regions of the target sequence, relative to the control (FIG. 7). Similarly, for the EMX1 target, EMX1-BNA-5 displayed improved specificity towards sequences bearing mutations in the PAM-proximal and especially PAM-distal regions, compared to the unmodified crRNA (FIG. 8).

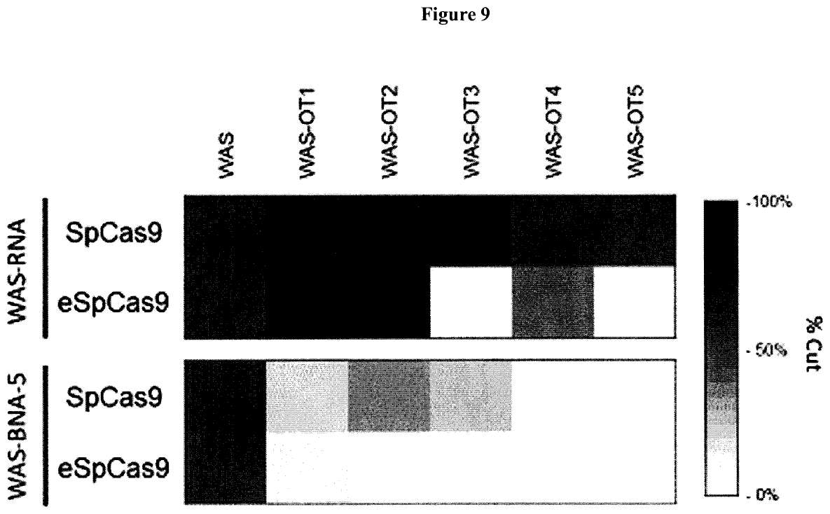

Recently, engineered variants of Cas9 displaying improved specificity have been developed (Slaymaker I M, et al. (2016) Rationally engineered Cas9 nucleases with improved specificity. Science 351(6268):84-88; Kleinstiver B P, et al. (2016) High-fidelity CRISPR-Cas9 nucleases with no detectable genome-wide off-target effects. Nature 529(7587):490-495). To examine if the BNA^{NC}-substituted crRNAs could be used in conjunction with these variants to further improve specificity, we first profiled the activity of eSpCas9, a Cas9 variant with substitutions that reduce interactions with the non-complementary DNA strand (Slaymaker I M, et al. (2016) Rationally engineered Cas9 nucleases with improved specificity. Science 351(6268):84-88), using our WAS and EMX1 on- and off-target sequences. For the WAS gene set, we found that eSpCas9 reduced cleavage of several off-target sequences in vitro, but had little effect on others (WAS-OT1, 2, or 4 (FIG. 9)). In contrast, eSpCas9 abolished all off-target cleavage of the EMX1 sequences using the unmodified crRNA (not shown). Next, we repeated the cleavage assay of the WAS on- and off-target sequences using eSpCas9 in combination with the WAS-BNA-5 crRNA. We selected this modified crRNA since its specificity improvements were complementary to those of eSpCas9 with the unmodified crRNA. Strikingly, we found that the combination was synergistic, resulting in elimination of virtually all off-target activity (WAS-OT1 was still cleaved, but at a reduced level) (FIG. 9). These results suggest that bridged nucleic acid-modified crRNAs can be used to further boost the specificity of enhanced Cas9 variants.

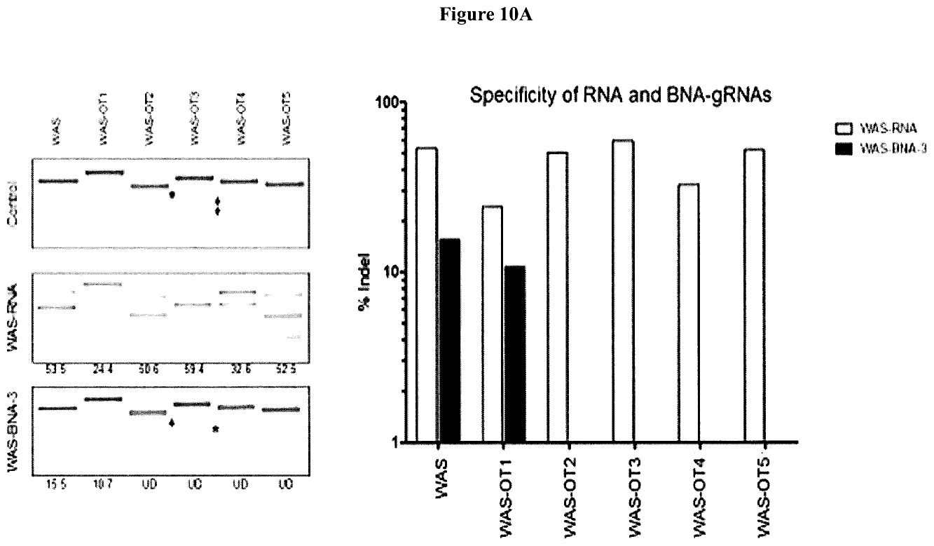

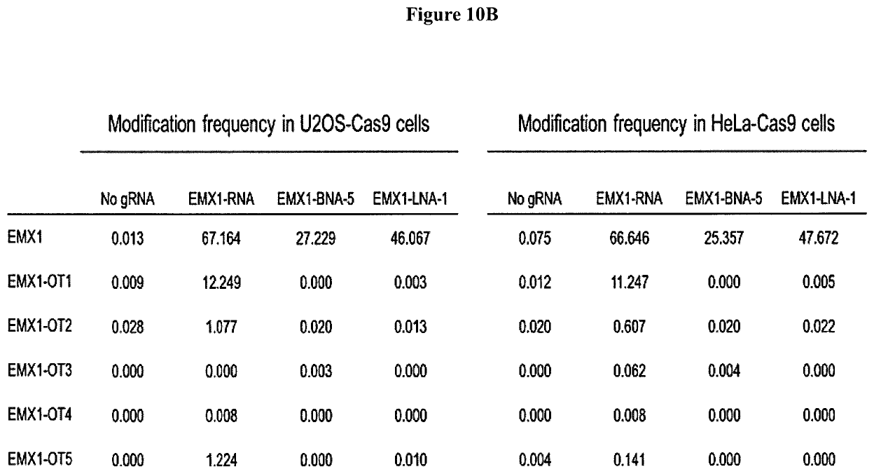

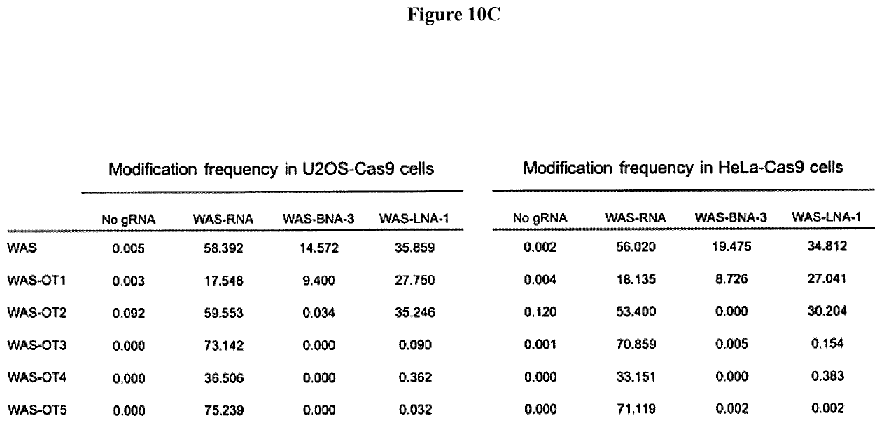

To test if the enhanced cleavage specificity of the BNA^{NC}-modified crRNAs observed in vitro translates into improved specificity in cells, we delivered either unmodified or BNA^{NC}- or LNA-modified crRNAs targeting the WAS and EMX1 loci at various doses into 293T cells stably expressing Cas9. T7 endonuclease assay results revealed that BNA^{NC}-modified crRNAs are active in cells, although higher molar concentrations are required to match the on-target modification rates of unmodified crRNAs (FIG. 10A). Using targeted high-throughput sequencing we compared indel modification of the on-target site and the 5 most abundant cellular off-target sites between unmodified and BNA^{NC}- or LNA-modified crRNAs, for both the WAS and EMX1 targets (FIGS. 10B and 10C). For the EMX1 target, we found that both EMX1-BNA5 and EMX-LNA-1 greatly reduced off-target cleavage compared to the unmodified crRNA (FIG. 10B). Similar results, mimicking our in vitro findings, were observed in the case of the WAS gene set (FIG. 10C). Importantly, while the LNA-modified crRNAs showed high on-target activity in cells (comparable to that of the unmodified crRNA), they showed weaker specificity improvements in the case of the WAS gene set, in agreement with our in vitro results FIG. 6. Collectively, these data establish bridged nucleic acid-modification of crRNAs as a new strategy to improve Cas9 DNA cleavage specificity in cells.

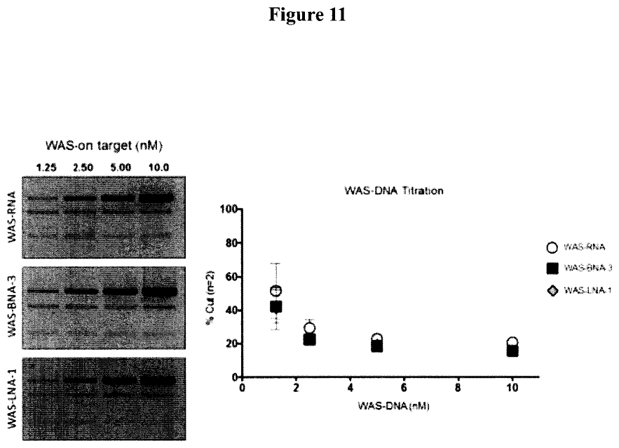

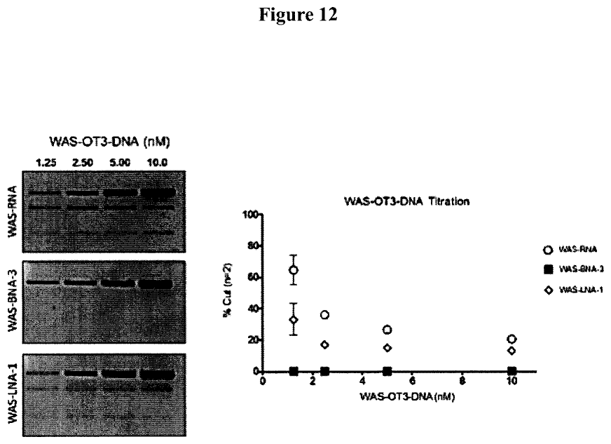

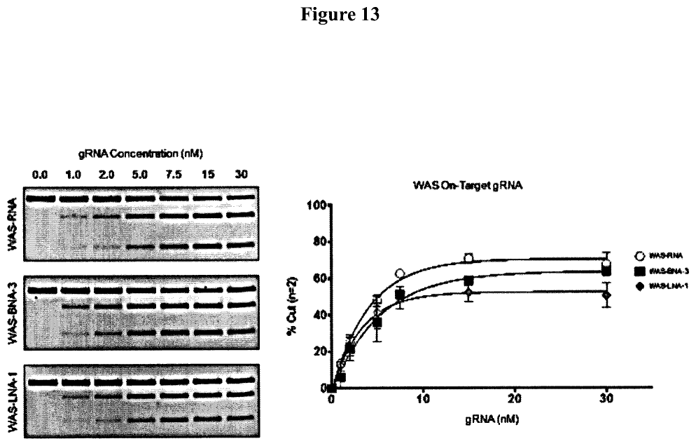

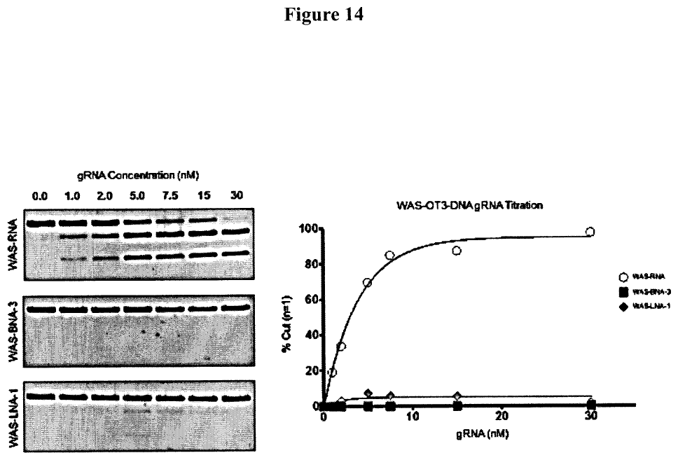

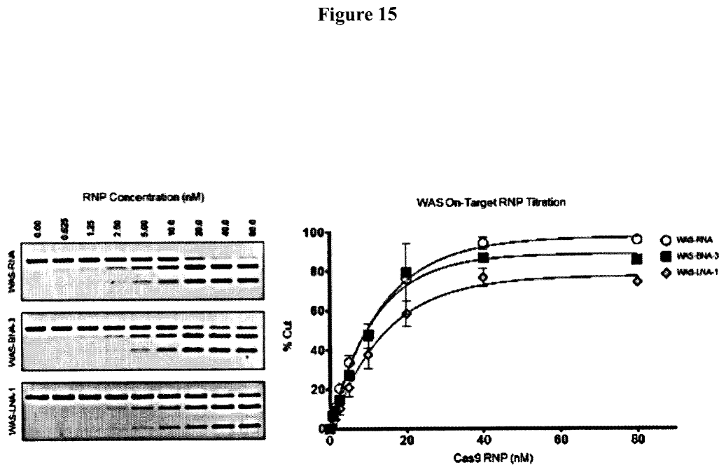

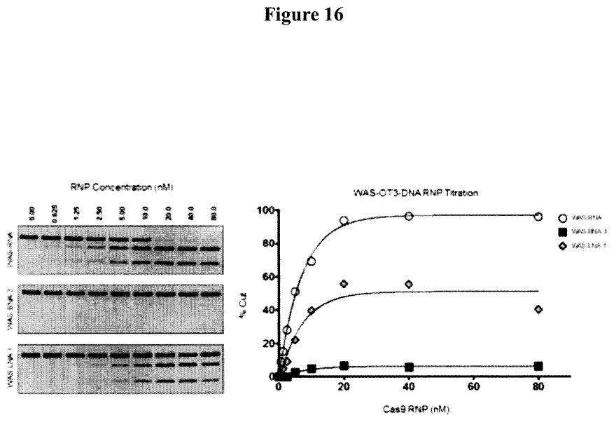

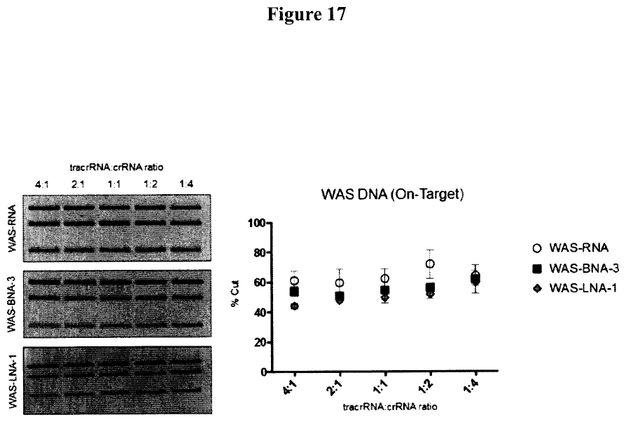

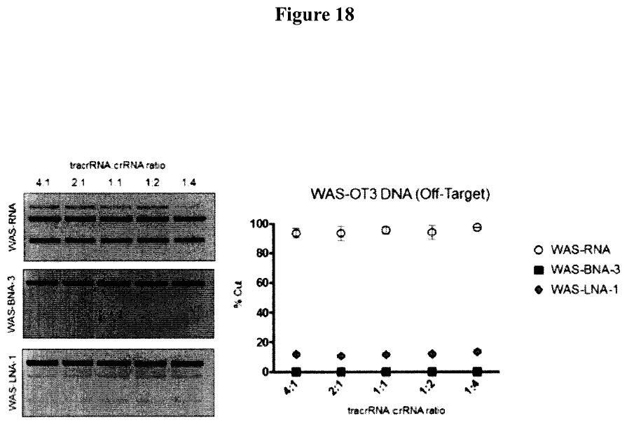

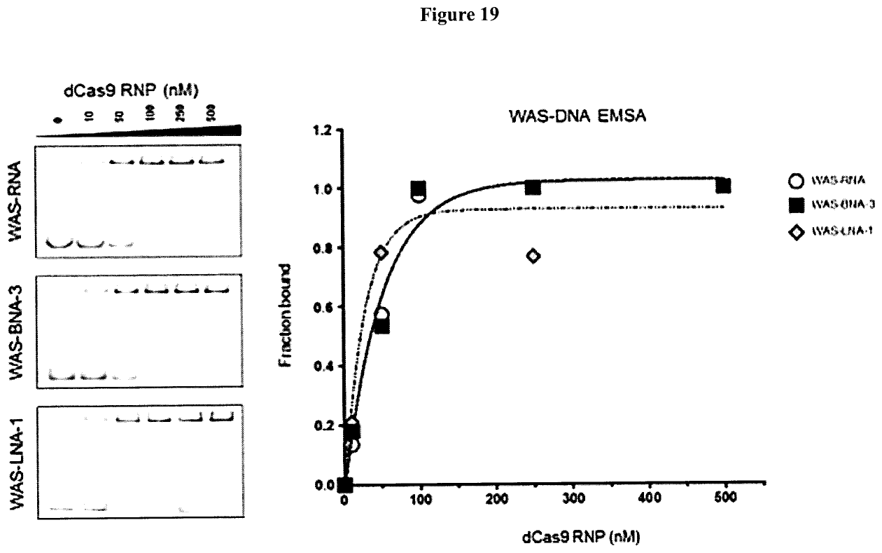

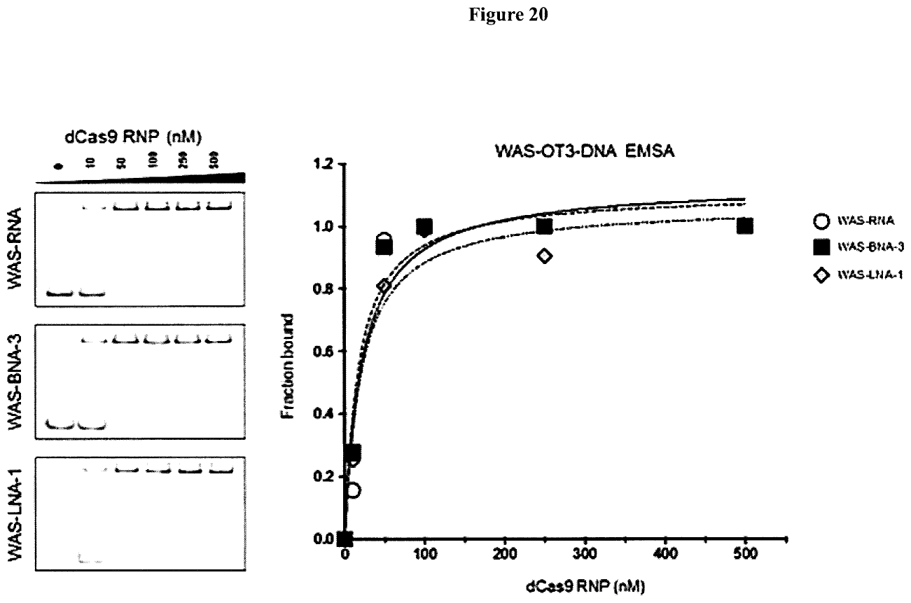

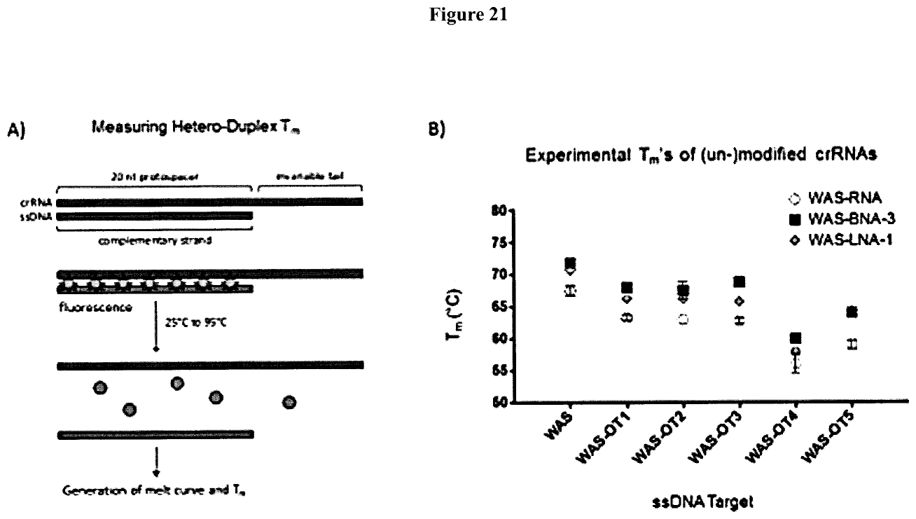

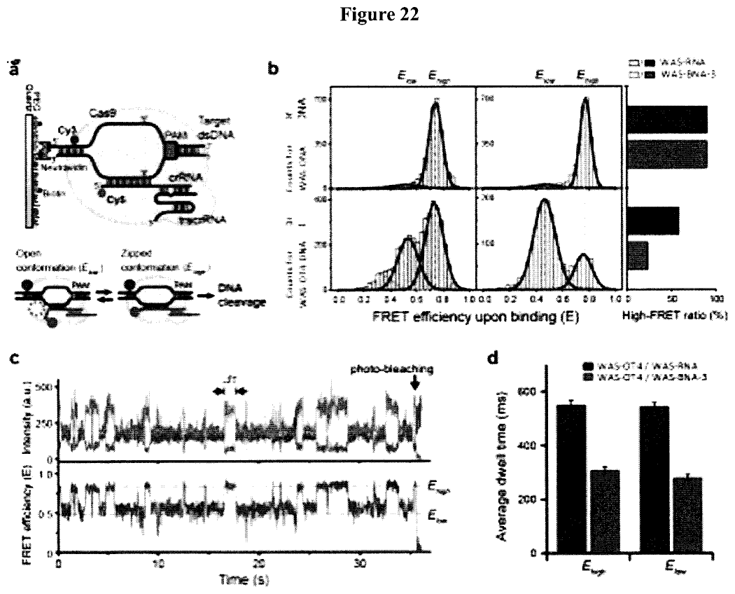

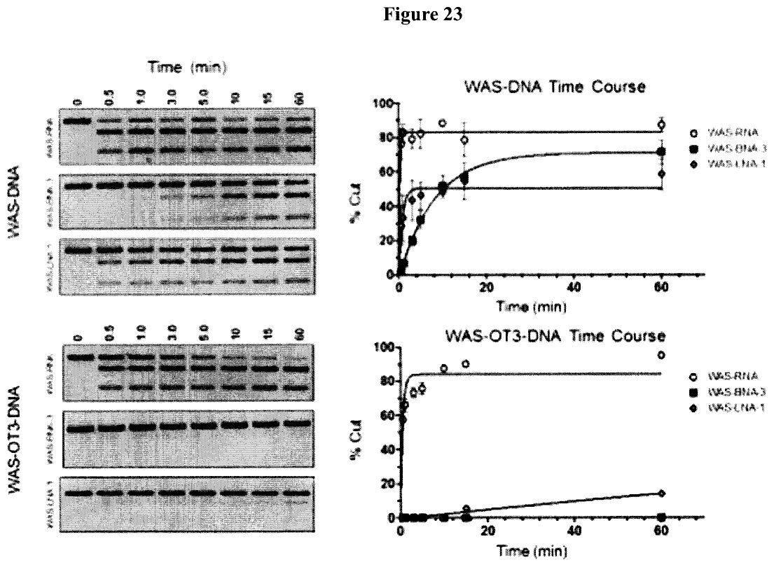

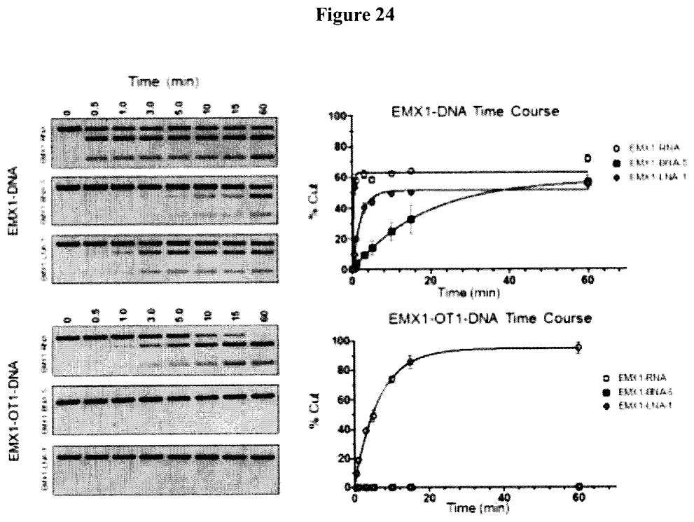

Global activity of the LNA and BNA(^{NC})-modified crRNAs were comparable to that of the unmodified crRNA over a wide range of DNA concentrations, gRNA concentrations, and RNP doses (FIG. 11 and FIG. 12 and FIG. 13 and FIG. 14 and FIG. 15 and FIG. 16). Five distinct stages in the Cas9 cleavage reaction can be resolved: tracRNA/crRNA loading, binding of the RNP to target DNA, DNA melting and PAM-proximal hybridization (“open conformation”), complete R-loop formation (“zipped conformation”), and structural rearrangement of the nuclease domain leading to cleavage (Lim Y, et al. (2016) Structural roles of guide RNAs in the nuclease activity of Cas9 endonuclease. Nat Commun 7:13350). To identify the mechanism underlying the improved specificity of BNA^{NC}-modified crRNAs, we studied their effect on each of these stages. Titrations of tracrRNA/crRNA revealed that both LNA and BNA^{NC}-modified crRNAs bind Cas9 similarly to their unmodified counterparts (FIG. 17 and FIG. 18). Moreover, electrophoretic mobility assay (EMSA) experiments showed no changes in binding to on- or off-target DNA sequences between Cas9 RNPs containing either unmodified or LNA or BNA^{NC}-modified crRNAs (FIG. 19 and FIG. 20). Next, we next sought to study changes in crRNA/DNA hybridization. We found that crRNA/DNA target hybridization and stability (Tₘ) in the absence of Cas9 was slightly increased by incorporation of BNA^{NC} or LNA bases for both on- and off-target sequences (FIG. 21). To monitor changes in hybridization in the presence of Cas9 (FIG. 22), we employed a previously described single-molecule fluorescence resonance energy transfer (FRET) technique (Lim Y, et al. (2016) Structural roles of guide RNAs in the nuclease activity of Cas9 endonuclease. Nat Commun 7:13350). Using this technology, we were able to resolve 3 distinct crRNA/target DNA conformations present after equilibrium, consisting of bound, partially zipped (“open”), and fully-zipped states. Interestingly, we identified an increase in dwell time in the open conformation occurring on both on- and off-target sequences using WAS-BNA-3 (relative to control) (FIG. 22). Kinetic assays confirmed a slower cleavage rate of both on- and off-target sequences using this BNA^{NC}-modified crRNA, but that this was not the case for LNA-modified crRNAs (FIG. 23 and FIG. 24). Consistent with improved specificity, we found that the proportion of molecules in the productive zipped state was decreased on off-target sequences using WAS-BNA-3 compared to the unmodified crRNA; this parameter was unaffected in the case of the on-target WAS sequence. Taken together, these results indicate that specificity improvements imparted by BNA¹ incorporation likely stem from delayed reaction kinetics coupled to an impaired ability to form a productive zipped conformation on off-target sequences. Moreover, the delayed reaction kinetics observed with the BNA^{NC}-modified crRNAs could explain the lower on-target cleavage activity observed in cells but not observed in vitro.

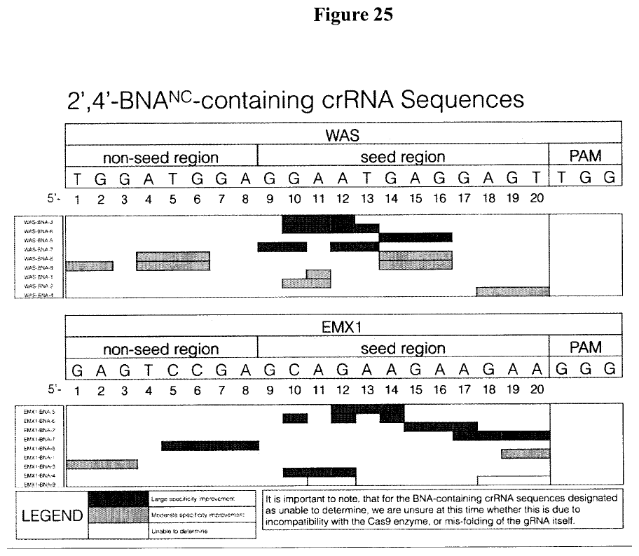

It is interesting to speculate about the interactions between Cas9 and BNA^{NC} bases that result in a reduced ability to form a productive zipped conformation on off-target sequences. Structural studies have shown that R-loop formation begins through hybridization of a ˜10 bp ‘seed’ sequence (bases 11-20 from the 5′ end) on the crRNA which is pre-ordered for interrogation as an A-form helix (Jiang F, Zhou K, Ma L, Gressel S, & Doudna J A (2015) STRUCTURAL BIOLOGY. A Cas9-guide RNA complex preorganized for target DNA recognition. Science 348(6242):1477-1481). Extensive hydrogen bonding between Cas9 and the phosphates and 2′ hydroxyl groups of the seed nucleotides enforces this helical conformation, and Cas9 is highly sensitive to mismatches within this region (Jiang F, Zhou K, Ma L, Gressel S, & Doudna J A (2015) STRUCTURAL BIOLOGY. A Cas9-guide RNA complex preorganized for target DNA recognition. Science 348(6242):1477-1481). In contrast, the PAM-distal region of the crRNA is maintained in a more disordered and mobile conformation by helical domain III (as compared to helical domain I) (Jiang F & Doudna J A (2017) CRISPR-Cas9 Structures and Mechanisms. Annu Rev Biophys 46:505-529). Because of this higher flexibility, Cas9 is more tolerant towards mismatches in this area of the guide sequence (Jiang F & Doudna J A (2017) CRISPR-Cas9 Structures and Mechanisms. Annu Rev Biophys 46:505-529). Locked nucleic acids adopt A-form helical conformations (Vester B & Wengel J (2004) LNA (locked nucleic acid): high-affinity targeting of complementary RNA and DNA. Biochemistry 43(42):13233-13241). Since the most general specificity improvements arise from BNA^{NC}-substitutions in the middle of the crRNA sequence (between the seed and PAM-distal regions) (FIG. 25), without being bound by theory, it is possible that BNA^{NC}s serve to functionally extend the length of the ordered A-form seed sequence, improving mismatch discrimination especially in the PAM-distal region.

Alternatively, the enhanced off-target discrimination of BNA^{NC}-modified crRNAs could be due formation of crRNA/off-target DNA hybridization geometries that can no longer be spatially accommodated by Cas9. Our data with LNA support this model, as demonstrated when replacement of BNA bases in WAS-BNA-3 and EMX1-BNA-5 with LNA bases also improves Cas9 specificity, but to a lesser extent (FIG. 5 and FIG. 6). This finding has several important implications. First, since LNA bases are in fact more conformationally restricted then BNA^{NC} bases (Rahman S M, et al. (2008) Design, synthesis, and properties of 2′,4′-BNA(^{NC}): a bridged nucleic acid analogue. J Am Chem Soc 130(14):4886-4896) (which are in turn more restricted than standard RNA bases), it is likely that the specificity improvements are not driven exclusively by a conformational mechanism. Furthermore, it suggests that other features of the BNA^{NC} bases, such as their larger steric bulk or chemical substituents (e.g. the nitrogen atom) likely play a role in improving mismatch discrimination. For example, increased steric bulk could induce geometric distortions that prevent accommodation of nearby mismatched base pairs, preventing transition to a zipped conformation. Furthermore, the nitrogen atom within the BNA^{NC} base has been reported to lower repulsion between negatively charged phosphate backbones on opposing DNA strands (Rahman S M, et al. (2008) Design, synthesis, and properties of 2′,4′-BNA(^{NC}): a bridged nucleic acid analogue. J Am Chem Soc 130(14):4886-4896), which could also impact hybridization. Further structural and crystallographic work will be needed to fully elucidate the details of the mechanism.

Methods

Chemical Reagents and Oligonucleotides. All chemicals were purchased from Sigma Aldrich. DNA oligonucleotides and tracrRNA were purchased from Integrated DNA Technologies (IDT). Unmodified crRNA and crRNA containing bridged nucleic acids were purchased from BioSynthesis Inc., while crRNA containing locked nucleic acids (LNAs) were purchased from Exiqon. eSpCas9 nuclease was purchased from Sigma Aldrich.

CRISPR/Cas9 Sequences:

67-mer tracrRNA:
[SEQ ID NO 1]
AGCAUAGCAAGUUAAAAUAAGGCUAGUCCGUUAUCAACUUGAAAAAG
UGGCACCGAGUCGGUGCUUU

36-mer crRNA:
16-mer tail of cRNA—GUUUUAGAGCUAUGCU—[SEQ ID NO 2]. This is the invariable 3′ tail used for the crRNAs; the 20 nt upstream sequence being dependent upon the target.

WAS crRNA:
[SEQ ID NO 3]
UGGAUGGAGGAAUGAGGAGUGUUUUAGAGCUAUGCU
EMX1 crRNA:
[SEQ ID NO 4]
GAGUCCGAGCAGAAGAAGAAGUUUUAGAGCUAUGCU

FIG. 3 shows the 20 nt upstream sequences for the WAS-BNA and WAS-LNA analogues. e.g. crRNA for WAS-BNA-1: 5′-UGGAUGGAGGAAUGAGGAGUGUUUUAGAGCUAUGCU-3′(wherein the bolded “A” at position 11 indicates a BNA substitution [SEQ ID NO 5]. In other words, FIG. 3 illustrates the first 20 nt from the 5′ end of each crRNA, and each crRNA then has the same invariable 3′ tail, which is SEQ ID NO 2.

FIG. 4 shows the 20 nt upstream sequences for the EMX-BNA and EMX-LNA analogues. e.g. crRNA for EMX-BNA-1: 5′-GAGUCCGAGCAGAAGAAGAAGUUUUAGAGCUAUGCU-3′(wherein the bolded “A's” at positions 19 and 20 indicates BNA substitutions [SEQ ID NO 6]. In other words, FIG. 4 illustrates the first 20 nt from the 5′ end of each crRNA, and each crRNA then has the same invariable 3′ tail, which is SEQ ID NO 2.

Plasmids/cloning. On- and off-target sequences were cloned into the XbaI and HindIII sites of pUC19 to generate in vitro cleavage assay plasmid templates. Cas9 expressed from pET-NLS-Cas9-6×His (Addgene #62934) was used for all in vitro cleavage assay experiments. Site directed mutagenesis of pET-NLS-Cas9n-6×His (D10A) was performed using the Q5 Site Directed Mutagenesis Kit (NEB) to generate a pET-NLS-dCas9-6×His (D10A/H840A) expression construct. dCas9 expressed from pET-NLS-dCas9-6×His (D10A/H840A) was used for all electromobility shift assay (EMSA) experiments.

Expression and purification of S. pyogenes Cas9. E. coli Rosetta 2 cells were transformed with a plasmid encoding the S. pyogenes cas9 gene fused to an N-terminal 6×His-tag and NLS (Addgene #62934). The resulting bacterial strain was used to inoculate 5 mL of Luria-Bertani (LB) broth containing 50 μg/mL carbenicillin at 37° C. overnight. The cells were diluted 1:100 into the same growth medium and grown at 37° C. until an OD₆₀₀ of 0.6 was reached. The culture was incubated at 16° C. for 30 min after which isopropyl-ß-D-1-thiogalactopyranoside (IPTG) was added to a final concentration of 0.5 mM to induce Cas9 expression. After 16 h, cells were collected by centrifugation for 15 min at 2700×g and re-suspended in lysis buffer (20 mM Tris-Cl, pH 8.0, 250 mM NaCl, 5 mM imidazole, pH 8.0, 1 mM PMSF). The solution was incubated on ice for 30 min before proceeding. The cells were further lysed by sonication (30 s pulse-on and 60 s pulse-off for 7.5 min at 60% amplitude) with soluble lysate being obtained by centrifugation at 30 000×g for 30 min. The cell lysate containing Cas9 was injected into a HisTrap FF Crude column (GE Healthcare) attached to an AKTA Start System (GE Healthcare) and washed with wash buffer (20 mM Tris-Cl, pH 8.0, 250 mM NaCl, 10 mM imidazole, pH 8.0) until UV absorbance reached a baseline. Cas9 was eluted in elution buffer (20 mM Tris-Cl, pH 8.0, 250 mM NaCl, 250 mM imidazole, pH 8.0) in a single step. Eluted Cas9 was exchanged to storage buffer (20 mM HEPES-KOH, pH 7.5, 500 mM NaCl, 1 mM DTT) while being concentrated in a 100 kDa centrifugal filter (Pall). Concentrated Cas9 was flash-frozen in liquid nitrogen and stored in aliquots at −80° C. dCas9 was purified as described above.

In-vitro Cas9 cleavage assay. Plasmid templates for PCR were generated through ligation of annealed oligonucleotide WAS/EMX1 targets (Wang X, et al. (2014) Unbiased detection of off-target cleavage by CRISPR-Cas9 and TALENs using integrase defective lentiviral vectors. Nat. Biotechnol. 33(2):175-178) (Kim D, et al. (2016) Genome-wide target specificities of CRISPR-Cas9 nucleases revealed by multiplex Digenome-seq. Genome Res. 26:405-415) into HindIII/XbaI double-digested pUC19 (AddGene). On- and off-target substrate DNAs (referenced above) were generated through PCR with the plasmid templates and pUC19_fwd and pUC19_rev primers, then purified with QIAquick PCR Purification Kit (Qiagen). Equimolar amounts of tracrRNA (IDT) and crRNA (BioSynthesis) were heated at 95° C. for 10 min, and cooled to 25° C. over the course of 1 h to prepare guide RNAs (gRNAs). gRNAs containing BNA- and LNA-cRNAs were prepared as described above. 5 nM substrate DNAs were incubated with 150 nM Cas9 and 150 nM gRNA, or 15 nM Cas9 and 15 nM gRNA for 1 h at 37° C. in Cas9 cleavage buffer (5% glycerol, 0.5 mM EDTA, 1 mM DTT, 2 mM MgCl₂, 20 mM HEPES pH 7.5, 100 mM KCl), then purified with the MinElute PCR Purification Kit (Qiagen). Cleavage products were resolved on a 1% agarose gel and imaged on an Amersham Imager 600 (GE Healthcare). Cleavage assays using eSpCas9 (Sigma Aldrich) were performed as described above.

Electrophoretic mobility shift assay (EMSA). To prepare the 6-FAM labelled DNA substrate, target and non-target strands were mixed in a 1.5:1 molar ratio, incubated at 95° C. for 5 min, then cooled to 25° C. over the course of 1 h. DNA substrates were diluted to a working concentration of 200 nM in binding buffer (20 mM HEPES, pH 7.5, 250 mM KCl, 2 mM MgCl₂, 0.01% Triton X-100, 0.1 mg mL⁻¹ bovine serum albumin, 10% glycerol). gRNAs were prepared as described for in vitro cleavage assays. Nuclease-deficient Cas9 (dCas9) was incubated with gRNA in a 1:1 molar ratio for 10 min at 25° C. in binding buffer to form the ribonucleoprotein (RNP) complex. 50 nM substrate was incubated with 0, 10, 50, 100, 250 and 500 nM RNP for 10 min at 37° C. in binding buffer. Reactions were resolved on a 10% TBE polyacrylamide gel supplemented with 2 mM MgCl₂ in 1×TBE buffer supplemented with 2 mM MgCl₂ and imaged on a Typhoon laser gel scanner (GE Healthcare). EMSAs using BNA-containing gRNAs were performed as described above.

crRNA/Target DNA melting temperature measurement. Equimolar amounts of crRNA and complementary single-stranded DNA were mixed in duplex buffer to a final concentration of 2 μM. SYBR Green I was added to a final concentration of 1×. The solution was moved to a CFX96 Real Time System (BioRad) and incubated for 5 min at 95° C., then cooled to 25° C. at 0.1° C./s to anneal the DNA/RNA heteroduplex. The heteroduplex was then heated at 0.1° C./s to 95° C. with SYBR Green I fluorescence being measured every cycle to generate a melt-curve.

Cell culture. 293T cells were cultured in high glucose DMEM media with pyruvate (Gibco) supplemented with 10% FBS+1×pen/strep+1× glutamine (Gibco). U2OS-Cas9 cells were cultured in high glucose DMEM media with pyruvate (Gibco) supplemented with 10% FBS+5 μg/mL blasticidin S HCl (Gibco).

Generation of Cas9 Stable Cells. lentiCas9-Blast (Addgene #52962) viral particles were purchased from Addgene. On the day of infection, cells were trypsanized, counted and diluted to a working concentration of 50 000 cells/mL in DMEM-complete media supplemented with 10 μg/mL polybrene. Viral particles were serially diluted down to 1:500 from the original stock (2.5×10⁵ Tu/mL), with 500 μL of each dilution added to the corresponding wells of a 6-well plate. 1 mL of cell suspension was added to each well and incubated at 37° C. and 5% CO₂. 48 h after infection, selection was performed using DMEM-complete supplemented with 10 μg/mL Blasticidin S HCl (Gibco). After selection, cells stably expressing Cas9 were maintained in DMEM-complete containing 5 μg/mL Blasticidin S HCl.

Cationic Lipid Transfection of Cas9 RNPs. 293T cells were plated 24 h prior so that they would be 70% confluent at the time of transfection. Lipofectamine CRISPRMAX Cas9 Transfection Reagent (Thermo Fisher) was used for all RNP cationic lipid transfections. Active Cas9 RNP complexes were assembled as described for in vitro cleavage assays. Cas9 RNPs were incubated with CRISPRMAX according to the manufacturer's instructions, then added to the cells to a final concentration of 10 nM.

Cationic Lipid Transfection of gRNA into Stable Cell Lines. Cells stably expressing Cas9 were transfected with RNAiMAX and gRNA according to the manufacturers' instructions to a final concentration of 30 nM. Experiments involving BNA- and LNA-gRNA were performed as described above.

T7 Endonuclease I Assay:

Genomic DNA (gDNA) from transfected cells was extracted using a DNeasy kit (Qiagen) 48 h after transfection according to the manufacturer's instructions and was quantified using a NanoPhotometer NP80 (Implen) spectrophotometer. Amplicon specific primer pairs and 100 ng of gDNA was used to PCR amplify the desired target site, then purified with the QIAquick PCR Purification Kit (Qiagen). T7 endonuclease I (T7E1) digestion of the PCR products was performed as described by the manufacturer (NEB).

Next Generation Sequencing of Amplicons:

100 ng genomic DNA isolated from cells from each treatment (control, RNA Cas9 RNP, BNA Cas9 RNP and LNA Cas9 RNP) were amplified by PCR with 10 s 72° C. extension for 35 cycles with primers (target)_fwd and (target) rev and 2×Q5 Hot Start High Fidelity Master Mix in Q5 Reaction Buffer (NEB). PCR products were gel purified via MinElute Gel Purification Kit (Qiagen). Purified PCR product was amplified by PCR with primers N### and S### for 7 cycles with 2×Q5 Hot Start High Fidelity Master Mix in Q5 Reaction Buffer (NEB). Amplified control and treated DNA pools were purified with the GeneRead Size Selection Kit (Qiagen), quantified with the Qubit 2.0 Fluorometer (ThermoFisher), pooled in a 1:1 ratio and subjected to paired-end sequencing on an Illumina MiSeq.

Single-Molecule Measurement:

The following was adapted from Lim, Y. et al. (2016). Structural roles of guide RNAs in the nuclease activity of Cas9 endonuclease. Nature Communications. 7:1-8.

Coverslips and quartz glasses were passivated by polyethylene glycol to prevent samples from nonspecific binding on the glass surface. All imaging was performed at 37° C. with the following buffer composition: 100 mM NaCl, 50 mM Tris-HCl pH 7.9, 10 mM MgCl₂, 1 mM DTT and 0.1 mg ml-1 BSA. For smFRET experiments for

FRET histograms, the oxygen scavenger (2.7 U ml-1 of pyranose oxidase (Sigma-Aldrich), 7.5 U ml-1 of catalase (Sigma-Aldrich) and 0.4% (w/v) of b-D-glucose) and the triplet quencher (2 mM Trolox) were applied to the buffer to prevent the organic fluorophores from severe photo-fatigue. The FRET histograms were obtained from the images after 30 min incubation of Cas9:gRNA (2 nM Cas9, 30 nM gRNAs) with DNA. In case of experiments for single-molecule time traces, imaging was performed at room temperature in the same condition with the aforementioned description except for the oxygen scavenging system (1 mg ml-1 of glucose oxidase (Sigma-Aldrich), 0.04 mg ml-1 of catalase (Sigma-Aldrich) and 0.8% (w/v) of b-D-glucose) and the addition of 5% (v/v) of glycerol. The time traces were acquired intermittently during the incubation (from 0 min to 30 min) of Cas9:gRNA with DNA. In all single-molecule measurements, we constructed a flow chamber by assembling a microscope slide and a coverslip with double-sided tape and sealing with epoxy. We adopted rounded-holes on the slide as the inlet and outlet of solution exchange.

Library for High-Throughput Profiling:

Generation of pre-selection libraries for in vitro high-throughput specificity profiling experiments were performed as previously described. Briefly, 10 pmol of WAS or EMX1 lib oligonucleotides (listed below) were circularized through incubation with 100 units of CircLigase II ssDNA Ligase (Epicenter) in a total reaction volume of 20 μL for 16 h at 60° C. in 1× CircLigase II Reaction Buffer. The reaction was heat inactivated by incubation at 85° C. for 10 min. 5 pmol of the crude circular ssDNA was converted into concatemeric pre-selection libraries with the illustra TempliPhi Amplification Kit (GE Healthcare) according to the manufacturer's protocol. Concatemeric pre-selection libraries were quantified with the Qubit 2.0 Fluorometer. All pre-selection libraries used for high-throughput specificity profiling (slides 1-10 and 16-23) were generated using this protocol.

In Vitro High-Throughput Specificity Profiling:

High-throughput specificity profiling of unmodified and modified crRNAs was performed as previously described. Briefly, 200 nM of concatemeric pre-selection libraries were incubated with 1000 nM Cas9 and 1000 nM gRNA or 100 nM Cas9 and 100 nM gRNA in Cas9 cleavage buffer (NEB) for 20 min at 37° C. Pre-selection libraries were also separately incubated with 2 U of BspMI (NEB) in NEBuffer 3.1 for 1 h at 37° C. Cas9-digested and BspMI-digested library members were purified with the QiaQuick PCR Purification Kit (Qiagen) and ligated to 10 pmol adaptor1/2(#) (post-selection) or lib adapter 1/lib adapter 2 (pre-selection) with 1000 U of T4 DNA Ligase (NEB) in NEB T4 DNA Ligase Reaction Buffer for 16 h at room temperature. Adapter ligated DNA was purified using the QiaQuick PCR Purification Kit (Qiagen) and PCR amplified for 19-24 cycles with Q5 Hot Start High-Fidelity DNA Polymerase (NEB) in Q5 Reaction Buffer using primers PE2 short/sel PCR (post-selection) or primers lib seq PCR/lib fwd PCR (pre-selection). PCR products were gel purified and quantified using a Qubit 2.0 Fluorometer (ThermoFisher) and subject to single-read sequencing on an Illumina MiSeq. Pre-selection and post-selection sequencing data were analyzed as previously described. High-throughput specificity profiling experiments shown on slides 1-10 and 16-23 were performed using the above protocol.

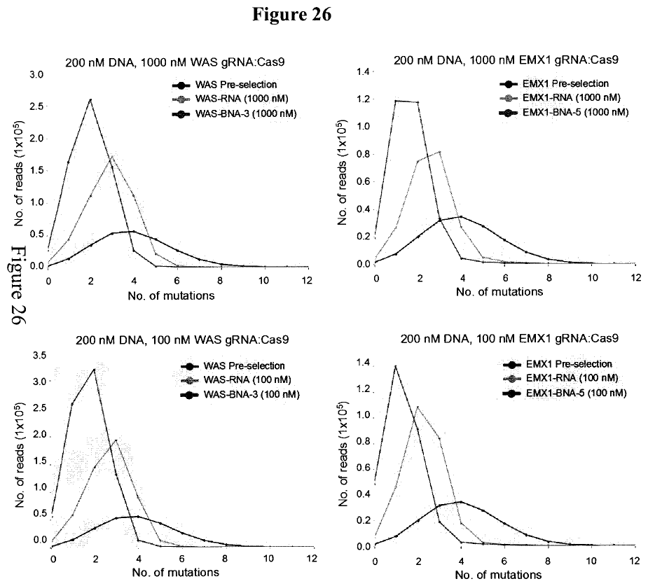

FIG. 26 demonstrates the ability of BNA-modified gRNAs to globally reduce in vitro off-target cleavage from a library of over 1012 potentially off-target sequences. The effect is most pronounced specifically with sequences showing ≥3 mismatches (relative to the on-target sequence).

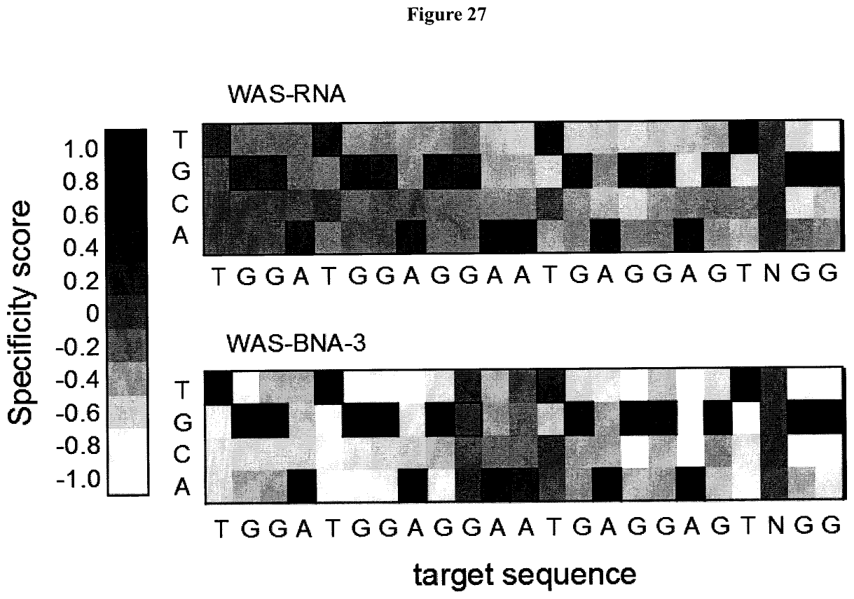

Experiments were performed using 200 nM pre-selection library WAS and 100 nM Cas9 RNP complex. Specificity scores were calculated using the formula: positive specificity score=(frequency of base pair at position[post-selection]−frequency of base pair at position[pre-selection])/(1−frequency of base pair at position[pre-selection]) and negative specificity score=(frequency of base pair at position[post-selection]−frequency of base pair at position[pre-selection])/(frequency of base pair at position[pre-selection. FIG. 27 demonstrates that BNA-modification of crRNAs improves specificity of the crRNA in multiple positions in the target WAS sequence (most significantly in the areas no overlapping with BNA substitutions—ie. adjacent to where the BNAs were incorporated).

Experiments were performed using 200 nM pre-selection library EMX1 and 100 nM Cas9 RNP complex. Specificity scores were calculated using the formula: positive specificity score=(frequency of base pair at position[post-selection]−frequency of base pair at position[pre-selection])/(1−frequency of base pair at position[pre-selection]) and negative specificity score=(frequency of base pair at position[post-selection]−frequency of base pair at position[pre-selection])/(frequency of base pair at position[pre-selection]).

This figure demonstrates that BNA-modification of crRNAs improves specificity of the crRNA in multiple positions in the target EMX1 sequence (most significantly in the areas no overlapping with BNA substitutions—ie. Adjacent to where the BNAs were incorporated).

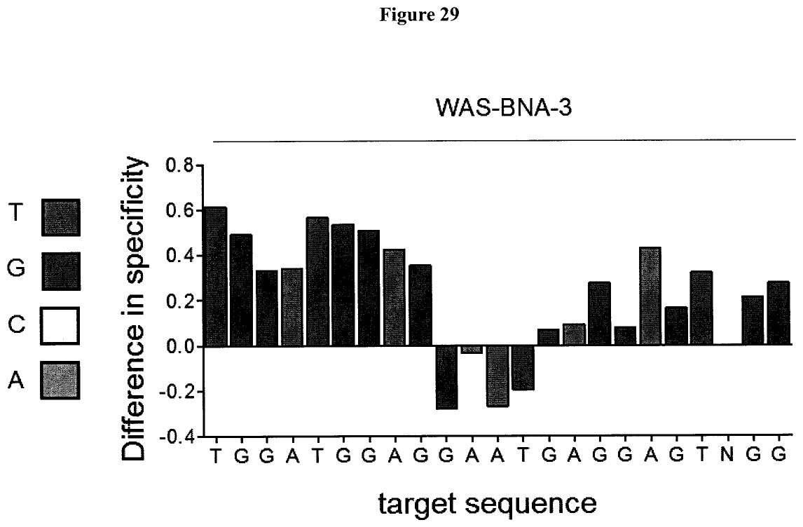

FIG. 29 shows a bar graph illustrating the quantitative differences in specificity score at each position along the 20 nucleotide Cas9 target site and 2 nucleotide PAM (of which of N of NGG is excluded) between the unmodified and BNA-modified crRNA for the WAS target. A score of zero indicates no change in specificity. Differences in specificity were calculated as: specificity score (BNA)−specificity score (RNA). Experiments shown were performed using 200 nM pre-selection library and 100 nM Cas9 RNP complex. This figure demonstrates that BNA-modification of crRNAs improves specificity of the crRNA in multiple positions in the target sequence (most significantly in the areas no overlapping with BNA substitutions—ie. Adjacent to where the BNAs were incorporated).

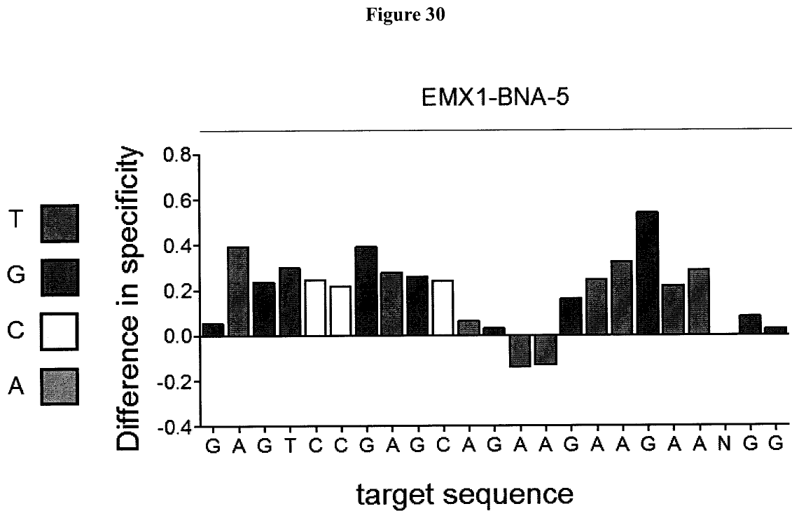

FIG. 30 is a bar graph illustrating the quantitative differences in specificity score at each position along the 20 nucleotide Cas9 target site and 2 nucleotide PAM (of which of N of NGG is excluded) between the unmodified and BNA-modified crRNA for the EMX1 target. A score of zero indicates no change in specificity. Differences in specificity were calculated as: specificity score (BNA)—specificity score (RNA). Experiments shown were performed using 200 nM pre-selection library and 100 nM Cas9 RNP complex. This figure demonstrates that BNA-modification of crRNAs improves specificity of the crRNA in multiple positions in the target sequence (most significantly in the areas no overlapping with BNA substitutions—ie. Adjacent to where the BNAs were incorporated).

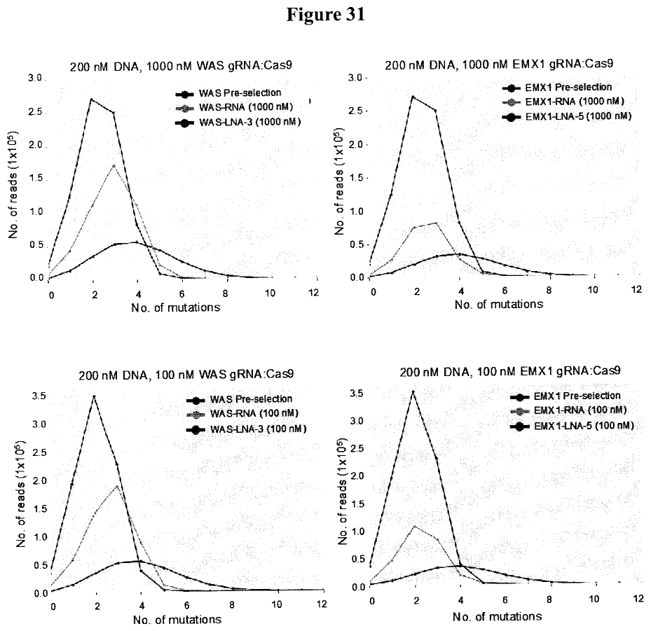

FIG. 31 shows distributions of mutations are shown for pre-selection (black) and post-selection (grey) libraries following in vitro high-throughput specificity profiling using either the WAS or EMX1 target sequence. In vitro selections were performed using 200 nM pre-selection library comprising ˜10¹² potential off-target sequences and 1000 nM or 100 nM Cas9 complexed with 1000 nM or 100 nM unmodified (light grey) or LNA-modified (dark grey) gRNA. Pre- and post-selection libraries were subject to high-throughput sequencing on an Illumina MiSeq platform. This figure demonstrates the ability of LNA-modified gRNAs to globally reduce in vitro off-target cleavage from a library of over 10¹² potentially off-target sequences. The effect is most pronounced specifically with sequences showing ≥3 mismatches (relative to the on-target sequence).

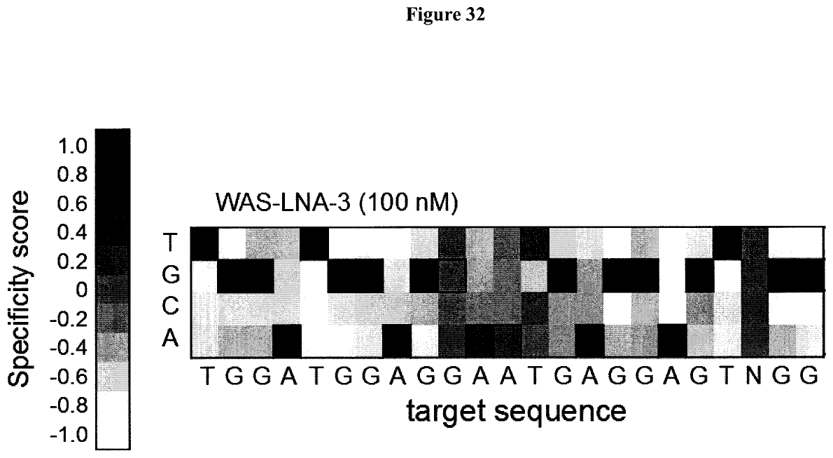

FIG. 32 shows a heat map showing DNA cleavage specificity scores following in vitro specificity profiling of ˜10¹² potential off-target sites using Cas9 complexed with an LNA-modified crRNA targeting the WAS sequence (listed below the heat map). Specificity scores of 1.0 (black) correspond to 100% enrichment for, while scores of −1.0 (white) correspond to 100% enrichment against a specific base-pair at a specific position along the target sequence. Black boxes denote the intended nucleotide at each position. Experiments were performed using 200 nM pre-selection library and 100 nM Cas9 RNP complex. Specificity scores were calculated using the formula: positive specificity score=(frequency of base pair at position[post-selection]−frequency of base pair at position[pre-selection])/(1−frequency of base pair at position[pre-selection]) and negative specificity score=(frequency of base pair at position[post-selection]−frequency of base pair at position[pre-selection])/(frequency of base pair at position[pre-selection]). This figure demonstrates that LNA-modification of crRNAs improves specificity of the crRNA in multiple positions in the target sequence (most significantly in the areas no overlapping with LNA substitutions—ie. Adjacent to where the LNAs were incorporated).

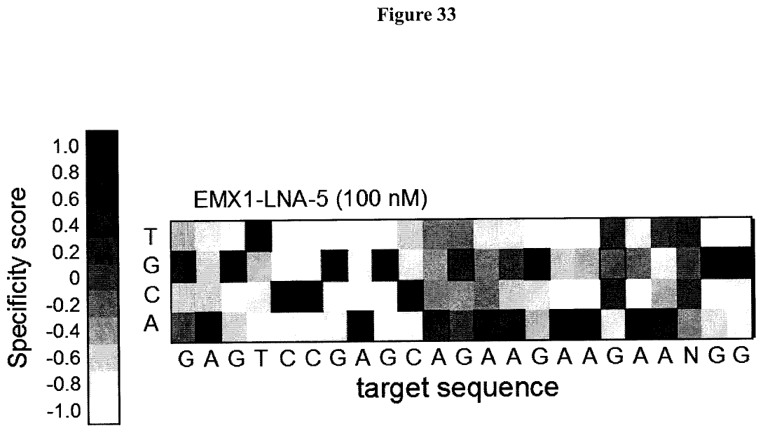

FIG. 33 shows a heat map showing DNA cleavage specificity scores following in vitro specificity profiling of ˜10¹² potential off-target sites using Cas9 complexed with an LNA-modified crRNA targeting the EMX1 sequence (listed below the heat map). Specificity scores of 1.0 (black) correspond to 100% enrichment for, while scores of −1.0 (white) correspond to 100% enrichment against a specific base-pair at a specific position along the target sequence. Black boxes denote the intended nucleotide at each position. Experiments were performed using 200 nM pre-selection library and 100 nM Cas9 RNP complex. Specificity scores were calculated using the formula: positive specificity score=(frequency of base pair at position[post-selection]−frequency of base pair at position[pre-selection])/(1−frequency of base pair at position[pre-selection]) and negative specificity score=(frequency of base pair at position[post-selection]−frequency of base pair at position[pre-selection])/(frequency of base pair at position[pre-selection]). This figure demonstrates that LNA-modification of crRNAs improves specificity of the crRNA in multiple positions in the target sequence (most significantly in the areas no overlapping with LNA substitutions—ie. Adjacent to where the LNAs were incorporated).

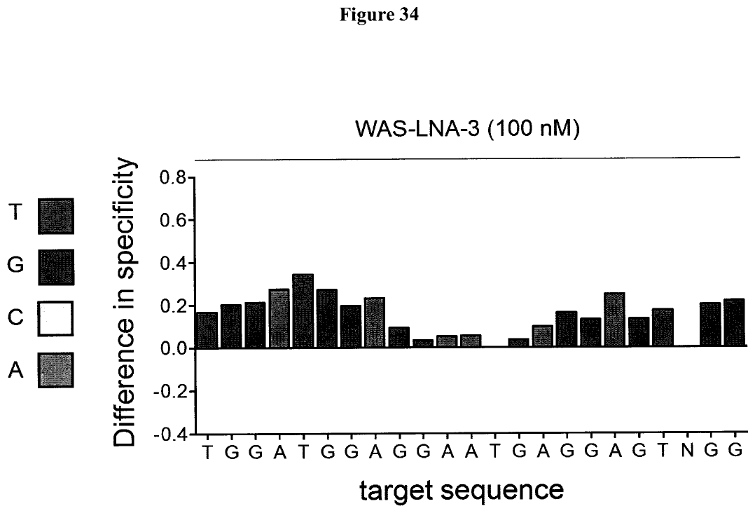

FIG. 34 shows a bar graph illustrating the quantitative differences in specificity score at each position along the 20 nucleotide Cas9 target site and 2 nucleotide PAM (of which of N of NGG is excluded) between the unmodified and LNA-modified crRNA for the WAS target. A score of zero indicates no change in specificity. Differences in specificity were calculated as: specificity score (LNA)−specificity score (RNA). Experiments shown were performed using 200 nM pre-selection library and 100 nM Cas9 RNP complex. This figure demonstrates that LNA-modification of crRNAs improves specificity of the crRNA in multiple positions in the target sequence (most significantly in the areas no overlapping with LNA substitutions—ie. Adjacent to where the LNAs were incorporated).

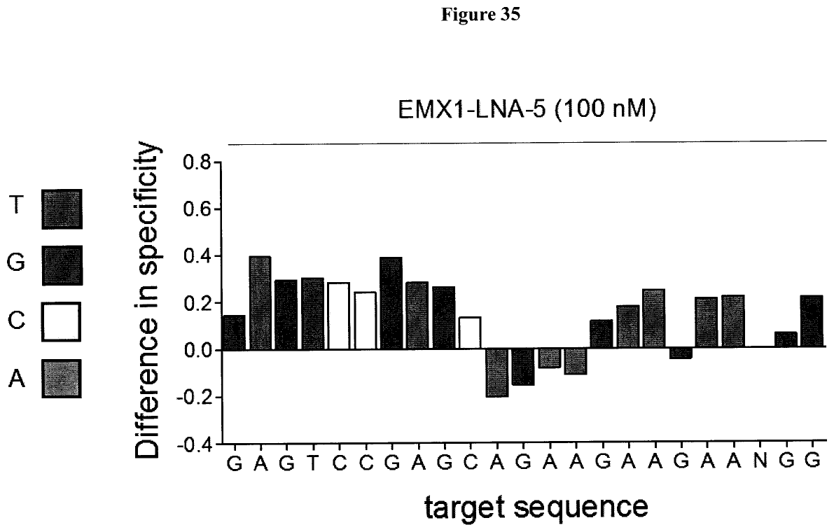

FIG. 35 shows a bar graph illustrating the quantitative differences in specificity score at each position along the 20 nucleotide Cas9 target site and 2 nucleotide PAM (of which of N of NGG is excluded) between the unmodified and LNA-modified crRNA for the EMX1 target. A score of zero indicates no change in specificity. Differences in specificity were calculated as: specificity score (LNA)−specificity score (RNA). Experiments shown were performed using 200 nM pre-selection library and 100 nM Cas9 RNP complex.

This figure demonstrates that LNA-modification of crRNAs improves specificity of the crRNA in multiple positions in the target sequence (most significantly in the areas no overlapping with LNA substitutions—ie. Adjacent to where the LNAs were incorporated).

BNA-Modified crRNAs

FIG. 36 illustrates the structures of RNA, DNA, 2′-O-methyl RNA, 2′-O-methyl RNA phosphonoacetate, 2′,4′-BNA (also referred to herein as “LNA”) and 2′,4′-BNA^{NC}[NMe](also referred to herein as “BNA”).

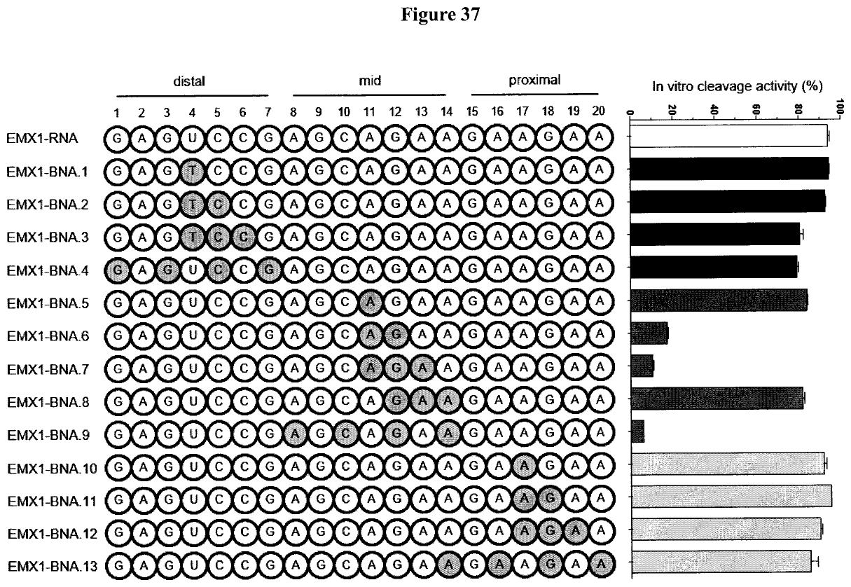

FIG. 37 shows a diagram outlining the sequences of unmodified and BNA-modified crRNAs targeting the EMX1 locus used in this study. BNA-modified nucleic acids are indicated by grey highlighting. In vitro cleavage assay results are shown to the right of the corresponding crRNA. 45 nM Cas9 and 45 nM unmodified or modified gRNA were pre-complexed for 10 min at 25° C. before addition of substrate DNA to a final concentration of 5 nM. Reactions were allowed to proceed for 1 h at 37° C. before purification via MinElute Purification Kit (Qiagen). Cleavage products were resolved on a 1% TBE agarose gel and processed with ImageJ. All experiments were performed in duplicate unless otherwise stated. This figure demonstrates the importance of BNA position along the crRNA as it relates to potential on-target activity.

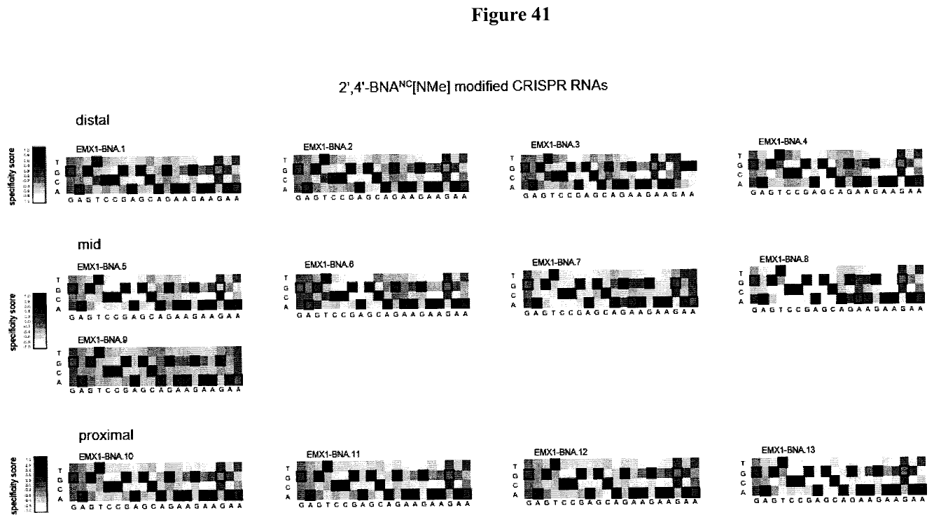

FIG. 41 shows heat maps showing DNA cleavage specificity scores following in vitro specificity profiling of ˜10¹² potential off-target sites using Cas9 complexed with BNA-modified crRNAs targeting the EMX1 sequence (listed below each heatmap). Specificity scores of 1.0 (black) correspond to 100% enrichment for, while scores of −1.0 (white) correspond to 100% enrichment against a specific base-pair at a specific position along the target sequence. Black boxes denote the intended nucleotide at each position. Experiments shown were performed using 5 nM pre-selection library and 30 nM Cas9 RNP complex. Specificity scores were calculated using the formula: positive specificity score=(frequency of base pair at position[post-selection]−frequency of base pair at position[pre-selection])/(1−frequency of base pair at position[pre-selection]) and negative specificity score=(frequency of base pair at position[post-selection]−frequency of base pair at position[pre-selection])/(frequency of base pair at position[pre-selection]).

This figure demonstrates that BNA-modification of crRNAs improves specificity of the crRNA in multiple positions in the target sequence (most significantly in the areas no overlapping with BNA substitutions—ie. Adjacent to where the BNAs were incorporated). Substitution of central or PAM-proximal positions have the most beneficial effects.

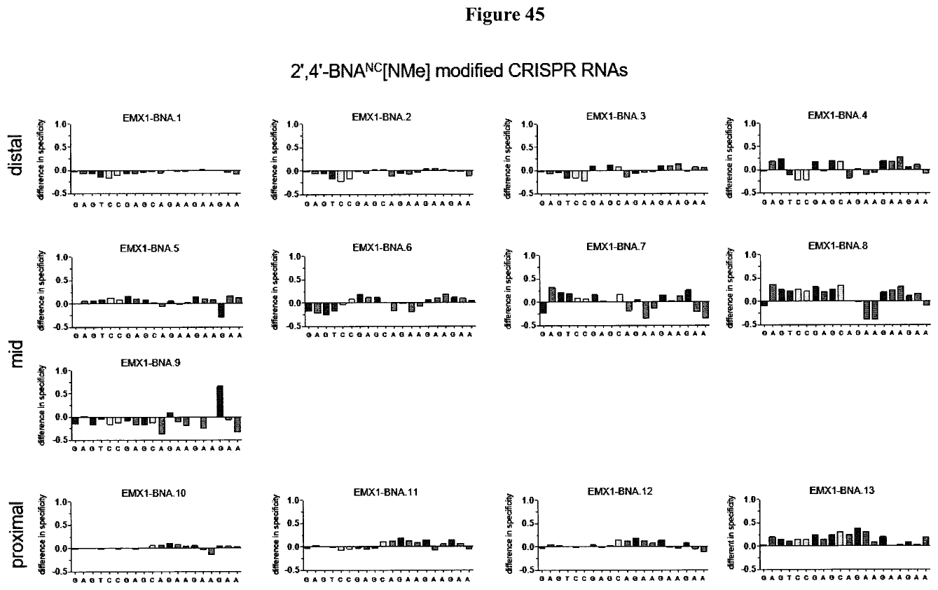

FIG. 45 shows bar graphs demonstrating the quantitative difference in specificity scores at each position along the 20 nucleotide target site and 2 nucleotide PAM (N of NGG excluded), between the unmodified and BNA-modified crRNA for EMX1 target sequences. A score of zero indicates no change in specificity. Differences in specificity were calculated as: specificity score(BNA)−specificity score(RNA). Experiments were performed using 5 nM pre-selection library and 30 nM Cas9 RNP complex.

This figure demonstrates that BNA-modification of crRNAs improves specificity of the crRNA in multiple positions in the target sequence (most significantly in the areas no overlapping with BNA substitutions—ie. Adjacent to where the BNAs were incorporated). Substitution of central or PAM-proximal positions have the most beneficial effects.

Methyl RNA-Modified crRNAs

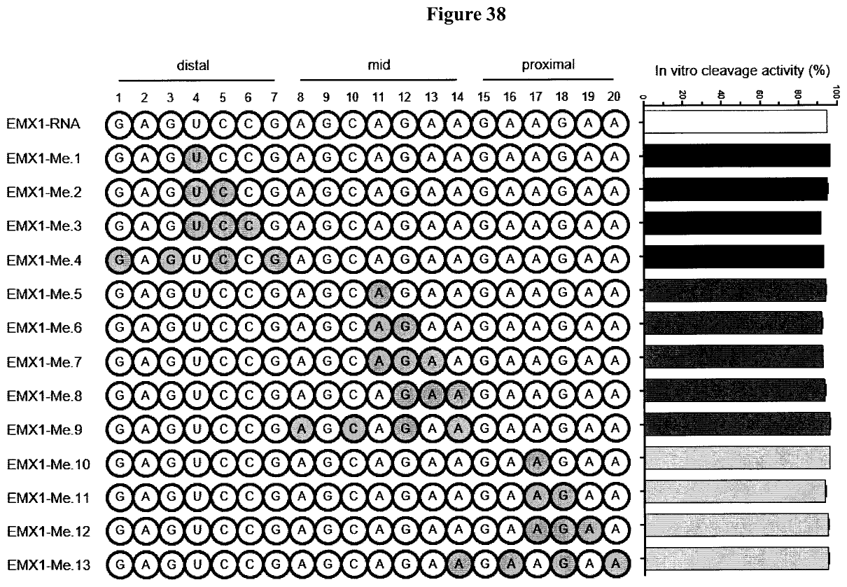

FIG. 38 shows a diagram outlining the sequences of unmodified and methyl RNA-modified crRNAs targeting the EMX1 locus used in this study. Methyl RNA-modified nucleic acids are indicated by grey highlighting. In vitro cleavage assay results are shown to the right of the corresponding crRNA. 45 nM Cas9 and 45 nM unmodified or modified gRNA were pre-complexed for 10 min at 25° C. before addition of substrate DNA to a final concentration of 5 nM. Reactions were allowed to proceed for 1 h at 37° C. before purification via MinElute Purification Kit (Qiagen). Cleavage products were resolved on a 1% TBE agarose gel and processed with ImageJ. All experiments were performed in duplicate unless otherwise stated. This figure demonstrates the importance of 2′OMe position along the crRNA as it relates to potential on-target activity.

Methyl Phosphonoacetate RNA-Modified crRNAs

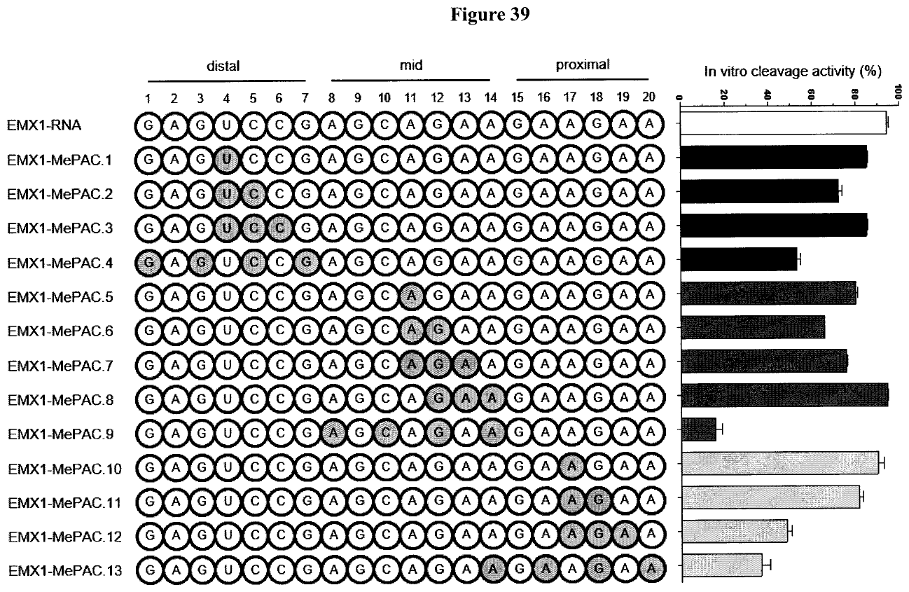

FIG. 39 shows a diagram outlining the sequences of unmodified and 2′O methyl phosphonoacetate RNA-modified crRNAs targeting the EMX1 locus used in this study. Methyl phosphonoacetate-modified nucleic acids are indicated by grey highlighting. In vitro cleavage assay results are shown to the right of the corresponding crRNA. 45 nM Cas9 and 45 nM unmodified or modified gRNA were pre-complexed for 10 min at 25° C. before addition of substrate DNA to a final concentration of 5 nM. Reactions were allowed to proceed for 1 h at 37° C. before purification via MinElute Purification Kit (Qiagen). Cleavage products were resolved on a 1% TBE agarose gel and processed with ImageJ. All experiments were performed in duplicate unless otherwise stated. This figure demonstrates the importance of 2′OMePAC position along the crRNA as it relates to potential on-target activity.

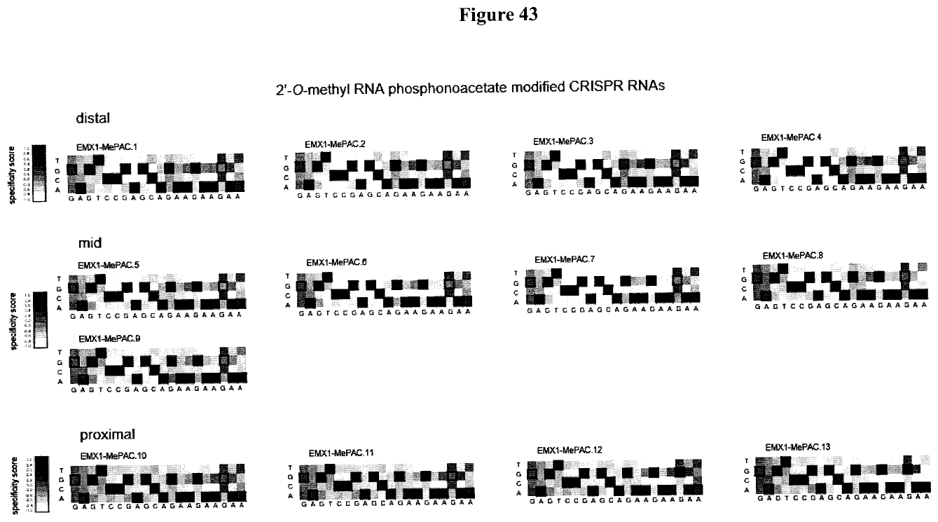

FIG. 43 shows heat maps showing DNA cleavage specificity scores following in vitro specificity profiling of ˜10¹² potential off-target sites using Cas9 complexed with 2′-O-methyl RNA phosphonoacetate-modified crRNAs targeting the EMX1 sequence (listed below each heatmap). Specificity scores of 1.0 (black) correspond to 100% enrichment for, while scores of −1.0 (white) correspond to 100% enrichment against a specific base-pair at a specific position along the target sequence. Black boxes denote the intended nucleotide at each position. Experiments shown were performed using 5 nM pre-selection library and 30 nM Cas9 RNP complex. Specificity scores were calculated using the formula: positive specificity score=(frequency of base pair at position[post-selection]−frequency of base pair at position[pre-selection])/(1−frequency of base pair at position[pre-selection]) and negative specificity score=(frequency of base pair at position[post-selection]−frequency of base pair at position[pre-selection])/(frequency of base pair at position[pre-selection]).

This figure demonstrates that 2′Ome PAC-modification of crRNAs improves specificity of the crRNA in multiple positions in the target sequence. Substitution of central or PAM-proximal positions have the most beneficial effects (some PAM distal also show improved specificity). Overall, positions 4-6, 11-13 (especially these, which overlap with the positions for the best BNA substitutions), and 14-20 show broad and global specificity improvement.

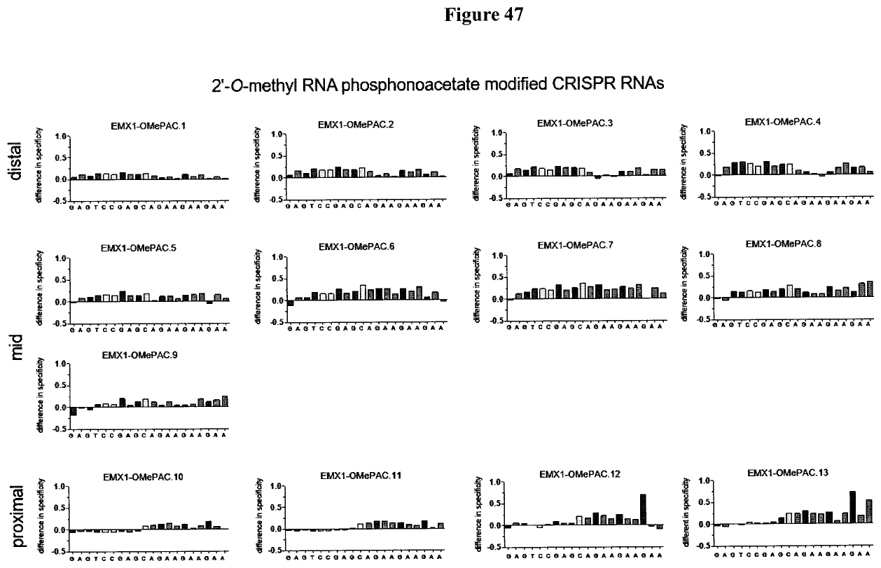

FIG. 47 shows bar graphs demonstrating the quantitative difference in specificity scores at each position along the 20 nucleotide target site and 2 nucleotide PAM (N of NGG excluded), between the unmodified and 2′-O-methyl RNA phosphonoacetate-modified crRNA for EMX1 target sequences. A score of zero indicates no change in specificity. Differences in specificity were calculated as: specificity score(2′-O-methyl RNA phosphonoacetate)−specificity score(RNA). Experiments were performed using 5 nM pre-selection library and 30 nM Cas9 RNP complex.

This figure demonstrates that 2′Ome PAC-modification of crRNAs improves specificity of the crRNA in multiple positions in the target sequence. Substitution of central or PAM-proximal positions have the most beneficial effects (some PAM distal also show improved specificity). Overall, positions 4-6, 11-13 (especially these, which overlap with the positions for the best BNA substitutions), and 14-20 show broad and global specificity improvement.

DNA-Modified crRNAs

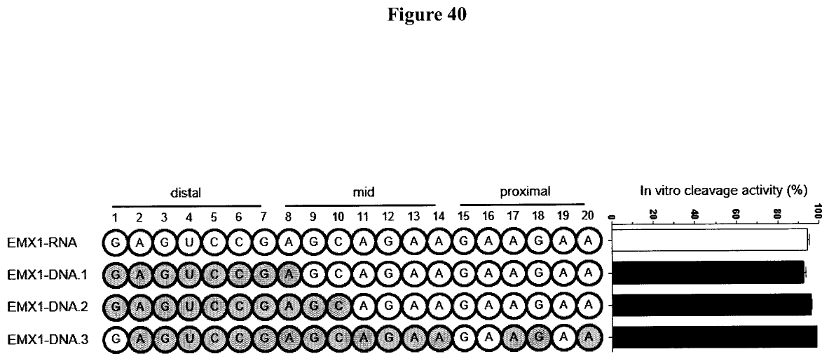

FIG. 40 shows a diagram outlining the sequences of unmodified and DNA-modified crRNAs targeting the EMX1 locus used in this study. DNA-modified nucleic acids are indicated by grey highlighting. In vitro cleavage assay results are shown to the right of the corresponding crRNA. 45 nM Cas9 and 45 nM unmodified or modified gRNA were pre-complexed for 10 min at 25° C. before addition of substrate DNA to a final concentration of 5 nM. Reactions were allowed to proceed for 1 h at 37° C. before purification via MinElute Purification Kit (Qiagen). Cleavage products were resolved on a 1% TBE agarose gel and processed with ImageJ. All experiments were performed in duplicate unless otherwise stated. This figure demonstrates the importance of DNA position along the crRNA as it relates to potential on-target activity.

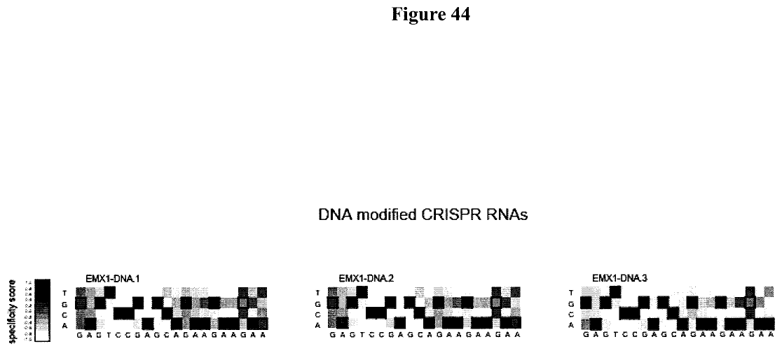

FIG. 44 shows heat maps showing DNA cleavage specificity scores following in vitro specificity profiling of ˜10¹² potential off-target sites using Cas9 complexed with DNA-modified crRNAs targeting the EMX1 sequence (listed below each heatmap). Specificity scores of 1.0 (black) correspond to 100% enrichment for, while scores of −1.0 (white) correspond to 100% enrichment against a specific base-pair at a specific position along the target sequence. Black boxes denote the intended nucleotide at each position. Experiments shown were performed using 5 nM pre-selection library and 30 nM Cas9 RNP complex. Specificity scores were calculated using the formula: positive specificity score=(frequency of base pair at position[post-selection]−frequency of base pair at position[pre-selection])/(1−frequency of base pair at position[pre-selection]) and negative specificity score=(frequency of base pair at position[post-selection]−frequency of base pair at position[pre-selection])/(frequency of base pair at position[pre-selection]).

This figure demonstrates that DNA-modification of crRNAs improves specificity of the crRNA in multiple positions in the target sequence Highly DNA-modified crRNAs show the best overall improvements (all acceptable positions previously shown to not disrupt on-target activity—Rueda et al., Nature Communications, 2017.

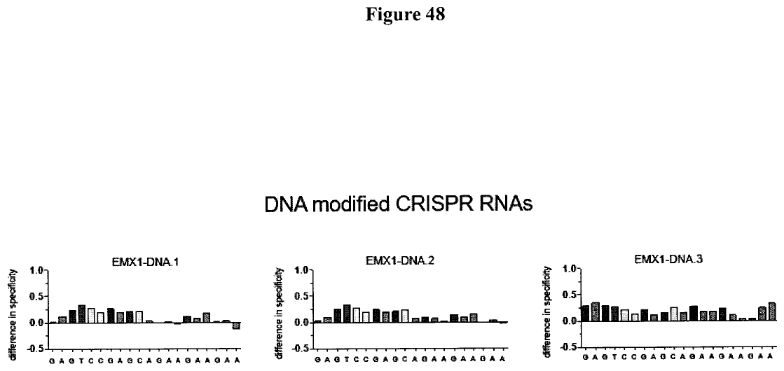

FIG. 48 shows bar graphs demonstrating the quantitative difference in specificity scores at each position along the 20 nucleotide target site and 2 nucleotide PAM (N of NGG excluded), between the unmodified and DNA-modified crRNA for EMX1 target sequences. A score of zero indicates no change in specificity. Differences in specificity were calculated as: specificity score(DNA)−specificity score(RNA). Experiments were performed using 5 nM pre-selection library and 30 nM Cas9 RNP complex.

This figure demonstrates that DNA-modification of crRNAs improves specificity of the crRNA in multiple positions in the target sequence Highly DNA-modified crRNAs show the best overall improvements (all acceptable positions previously shown to not disrupt on-target activity—Rueda et al., Nature Communications, 2017).

2′-O-Methyl RNA-Modified crRNAs

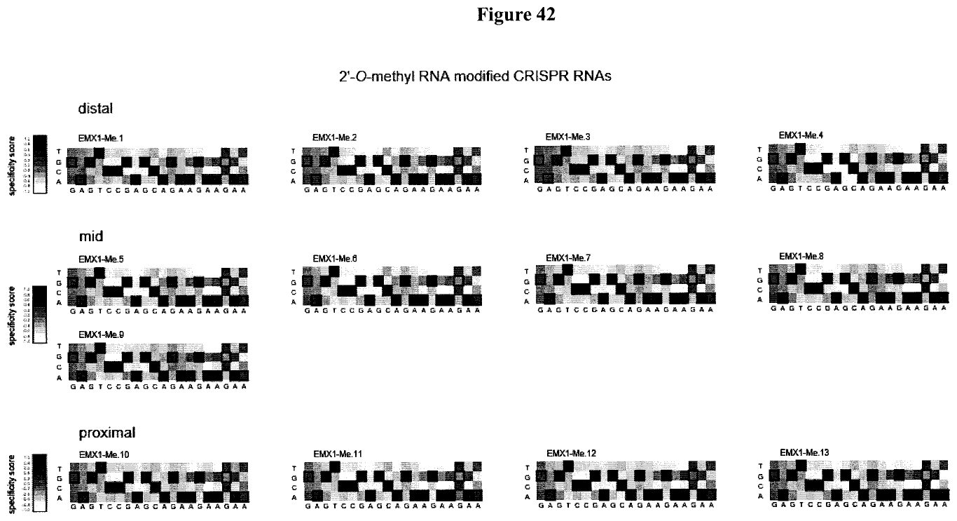

FIG. 42 shows heat maps showing DNA cleavage specificity scores following in vitro specificity profiling of ˜10¹² potential off-target sites using Cas9 complexed with 2′-O-methyl RNA-modified crRNAs targeting the EMX1 sequence (listed below each heatmap). Specificity scores of 1.0 (black) correspond to 100% enrichment for, while scores of −1.0 (white) correspond to 100% enrichment against a specific base-pair at a specific position along the target sequence. Black boxes denote the intended nucleotide at each position. Experiments shown were performed using 5 nM pre-selection library and 30 nM Cas9 RNP complex. Specificity scores were calculated using the formula: positive specificity score=(frequency of base pair at position[post-selection]−frequency of base pair at position[pre-selection])/(1−frequency of base pair at position[pre-selection]) and negative specificity score=(frequency of base pair at position[post-selection]−frequency of base pair at position[pre-selection])/(frequency of base pair at position[pre-selection]). This figure demonstrates that 2′Ome modification of crRNAs does not vastly improve Cas9 specificity (negative control).

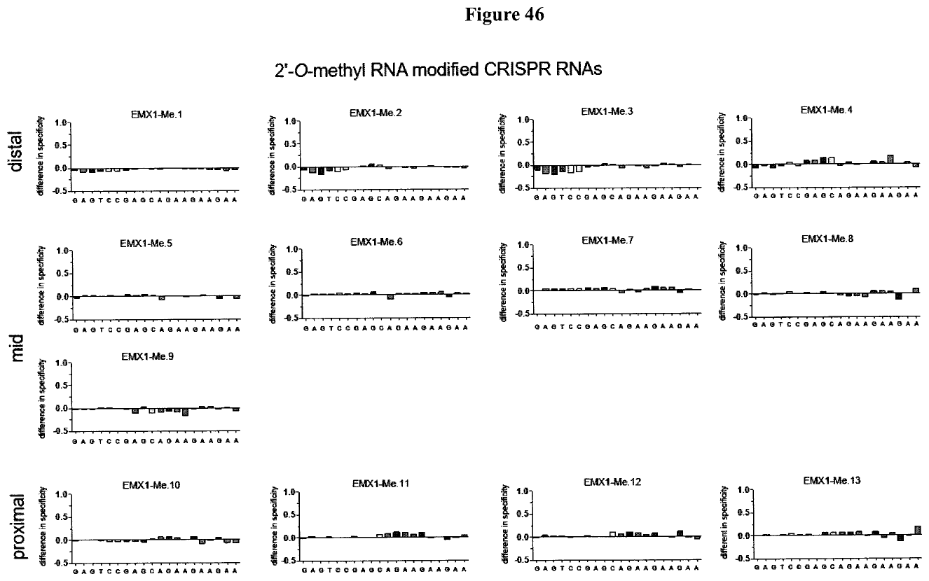

FIG. 46 shows bar graphs demonstrating the quantitative difference in specificity scores at each position along the 20 nucleotide target site and 2 nucleotide PAM (N of NGG excluded), between the unmodified and 2′-O-methyl RNA-modified crRNA for EMX1 target sequences. A score of zero indicates no change in specificity. Differences in specificity were calculated as: specificity score(2′-O-methyl RNA)−specificity score(RNA). Experiments were performed using 5 nM pre-selection library and 30 nM Cas9 RNP complex.

This figure demonstrates that 2′Ome modification of crRNAs does not vastly improve Cas9 specificity (negative control).

Oligonucleotides

The following sequences were ordered as ssDNA oligo, annealed together and ligated into the pUC19 plasmid backbone. All plasmids were ordered from AddGene (unless otherwise stated). All DNA oligonucleotides were ordered from Integrated DNA Technologies (IDT).

WAS_F
[SEQ ID NO 7]
GCCGAAGCTTCTTGGATGGAGGAATGAGGAGTGGGCTTCTAGAGGCC
WAS_R
[SEQ ID NO 8]
GGCCTCTAGAAGCCCACTCCTCATTCCTCCATCCAAGAAGCTTCGGC
WAS_SNP1_F
[SEQ ID NO 9]
GCCGAAGCTTCTTGGATGGAGGAATGAGGAGAGGGCTTCTAGAGGCC
WAS_SNP2_R
[SEQ ID NO 10]
GGCCTCTAGAAGCCCTCTCCTCATTCCTCCATCCAAGAAGCTTCGGC
WAS_SNP2_F
[SEQ ID NO 11]
GCCGAAGCTTCTTGGATGGAGGAATGAGGGGTGGGCTTCTAGAGGCC
WAS_SNP2_R
[SEQ ID NO 12]
GGCCTCTAGAAGCCCACCCCTCATTCCTCCATCCAAGAAGCTTCGGC
WAS_SNP3_F
[SEQ ID NO 13]
GCCGAAGCTTCTTGGATGGAGGAATGACGAGTGGGCTTCTAGAGGCC
WAS_SNP3_R
[SEQ ID NO 14]
GGCCTCTAGAAGCCCACTCGTCATTCCTCCATCCAAGAAGCTTCGGC
WAS_SNP4_F
[SEQ ID NO 15]
GCCGAAGCTTCTTGGATGGAGGAAAGAGGAGTGGGCTTCTAGAGGCC
WAS_SNP4_R
[SEQ ID NO 16]
GGCCTCTAGAAGCCCACTCCTCTTTCCTCCATCCAAGAAGCTTCGGC
WAS_SNP5_F
[SEQ ID NO 17]
GCCGAAGCTTCTTGGATGGAGGACTGAGGAGTGGGCTTCTAGAGGCC
WAS_SNP5_R
[SEQ ID NO 18]
GGCCTCTAGAAGCCCACTCCTCAGTCCTCCATCCAAGAAGCTTCGGC
WAS_SNP6_F
[SEQ ID NO 19]
GCCGAAGCTTCTTGGATGGAGAAATGAGGAGTGGGCTTCTAGAGGCC
WAS_SNP6_R
[SEQ ID NO 20]
GGCCTCTAGAAGCCCACTCCTCATTTCTCCATCCAAGAAGCTTCGGC
WAS_SNP7_F
[SEQ ID NO 21]
GCCGAAGCTTCTTGGATGGTGGAATGAGGAGTGGGCTTCTAGAGGCC
WAS_SNP7_R
[SEQ ID NO 22]
GGCCTCTAGAAGCCCACTCCTCATTCCACCATCCAAGAAGCTTCGGC
WAS_SNP8_F
[SEQ ID NO 23]
GCCGAAGCTTCTTGGATTGAGGAATGAGGAGTGGGCTTCTAGAGGCC
WAS_SNP8_R
[SEQ ID NO 24]
GGCCTCTAGAAGCCCACTCCTCATTCCTCAATCCAAGAAGCTTCGGC
WAS_SNP9_F
[SEQ ID NO 25]
GCCGAAGCTTCTTGGGTGGAGGAATGAGGAGTGGGCTTCTAGAGGCC
WAS_SNP9_R
[SEQ ID NO 26]
GGCCTCTAGAAGCCCACTCCTCATTCCTCCACCCAAGAAGCTTCGGC
WAS_SNP10_F
[SEQ ID NO 27]
GCCGAAGCTTCTTCGATGGAGGAATGAGGAGTGGGCTTCTAGAGGCC
WAS_SNP10_R
[SEQ ID NO 28]
GGCCTCTAGAAGCCCACTCCTCATTCCTCCATCGAAGAAGCTTCGGC
WAS_SNP11_F
[SEQ ID NO 29]
GCCGAAGCTTCTAGGATGGAGGAATGAGGAGTGGGCTTCTAGAGGCC
WAS_SNP11_R
[SEQ ID NO 30]
GGCCTCTAGAAGCCCACTCCTCATTCCTCCATCCTAGAAGCTTCGGC
EMX1_F
[SEQ ID NO 31]
GCCGAAGCTTCTGAGTCCGAGCAGAAGAAGAAGGGCTTCTAGAGGCC
EMX1_R
[SEQ ID NO 32]
GGCCTCTAGAAGCCCTTCTTCTTCTGCTCGGACTCAGAAGCTTCGGC
EMX1_SNP1_F
[SEQ ID NO 33]
GCCGAAGCTTCTGAGTCCGAGCAGAAGAAGAGGGGCTTCTAGAGGCC
EMX1_SNP2_R
[SEQ ID NO 34]
GGCCTCTAGAAGCCCCTCTTCTTCTGCTCGGACTCAGAAGCTTCGGC
EMX1_SNP2_F
[SEQ ID NO 35]
GCCGAAGCTTCTGAGTCCGAGCAGAAGAATAAGGGCTTCTAGAGGCC
EMX1_SNP2_R
[SEQ ID NO 36]
GGCCTCTAGAAGCCCTTATTCTTCTGCTCGGACTCAGAAGCTTCGGC
EMX1_SNP3_F
[SEQ ID NO 37]
GCCGAAGCTTCTGAGTCCGAGCAGAAGGAGAAGGGCTTCTAGAGGCC
EMX1_SNP3_R
[SEQ ID NO 38]
GGCCTCTAGAAGCCCTTCTCCTTCTGCTCGGACTCAGAAGCTTCGGC
EMX1_SNP4_F
[SEQ ID NO 39]
GCCGAAGCTTCTGAGTCCGAGCAGAGGAAGAAGGGCTTCTAGAGGCC
EMX1_SNP4_R
[SEQ ID NO 40]
GGCCTCTAGAAGCCCTTCTTCCTCTGCTCGGACTCAGAAGCTTCGGC
EMX1_SNP5_F
[SEQ ID NO 41]
GCCGAAGCTTCTGAGTCCGGGCAGAAGAAGAAGGGCTTCTAGAGGCC
EMX1_SNP5_R
[SEQ ID NO 42]
GGCCTCTAGAAGCCCTTCTTCTTCTGCCCGGACTCAGAAGCTTCGGC
EMX1_SNP6_F
[SEQ ID NO 43]
GCCGAAGCTTCTGAGTCCGAGTAGAAGAAGAAGGGCTTCTAGAGGCC
EMX1_SNP6_R
[SEQ ID NO 44]
GGCCTCTAGAAGCCCTTCTTCTTCTACTCGGACTCAGAAGCTTCGGC
EMX1_SNP7_F
[SEQ ID NO 45]
GCCGAAGCTTCTGAGTCCGGGCAGAAGAAGAAGGGCTTCTAGAGGCC
EMX1_SNP7_R
[SEQ ID NO 46]
GGCCTCTAGAAGCCCTTCTTCTTCTGCCCGGACTCAGAAGCTTCGGC
EMX1_SNP8_F
[SEQ ID NO 47]
GCCGAAGCTTCTGAGTCTGAGCAGAAGAAGAAGGGCTTCTAGAGGCC
EMX1_SNP8_R
[SEQ ID NO 48]
GGCCTCTAGAAGCCCTTCTTCTTCTGCTCAGACTCAGAAGCTTCGGC
EMX1_SNP9_F
[SEQ ID NO 49]
GCCGAAGCTTCTGAGGCCGAGCAGAAGAAGAAGGGCTTCTAGAGGCC
EMX1_SNP9_R
[SEQ ID NO 50]
GGCCTCTAGAAGCCCTTCTTCTTCTGCTCGGCCTCAGAAGCTTCGGC
EMX1_SNP10_F
[SEQ ID NO 51]
GCCGAAGCTTCTGCGTCCGAGCAGAAGAAGAAGGGCTTCTAGAGGCC
EMX1_SNP10_R
[SEQ ID NO 52]
GGCCTCTAGAAGCCCTTCTTCTTCTGCTCGGACGCAGAAGCTTCGGC
EMX1_SNP11_F
[SEQ ID NO 53]
GCCGAAGCTTCTTAGTCCGAGCAGAAGAAGAAGGGCTTCTAGAGGCC
EMX1_SNP11_R
[SEQ ID NO 54]
GGCCTCTAGAAGCCCTTCTTCTTCTGCTCGGACTAAGAAGCTTCGGC

The following primers were used to PCR amplify on- and off-target sequences for in vitro cleavage assays.

SEQUENCE LISTING

pUC19_fwd [SEQ ID NO 55] GCGACACGGAAATGTTGAATACTCAT
pUC19_rev [SEQ ID NO 56] CAGCGAGTCAGTGAGCGA
WAS_lib-
/5Phos/AACACANNNNC*C*NA*C*T*C*C*T*C*A*T*T*C*C*T*C*C*A*T*C*C*A*NN
NNACCTG CCGAGAACAC [SEQ ID NO 57]
EMX1_lib-
/5Phos/TCTTCTNNNNC*C*NT*T*C*T*T*C*T*T*C*T*G*C*T*C*G*G*A*C*T*C*NN
NNACCTGCCGAGTCTTCT [SEQ ID NO 58]
adaptor1(1) - AAT GAT ACG GCG ACC ACC GAG ATC TAC ACT CTT TCC CTA CAC
GAC GCT CTT CCG ATC TAC TGT [SEQ ID NO 59]
adaptor1(2) - AAT GAT ACG GCG ACC ACC GAG ATC TAC ACT CTT TCC CTA CAC
GAC GCT CTT CCG ATC TCT GAA [SEQ ID NO 60]
adaptor1(3) - AAT GAT ACG GCG ACC ACC GAG ATC TAC ACT CTT TCC CTA CAC
GAC GCT CTT CCG ATC TTG ACT [SEQ ID NO 61]
adaptor1(4) - AAT GAT ACG GCG ACC ACC GAG ATC TAC ACT CTT TCC CTA CAC
GAC GCT CTT CCG ATC TTG CAA [SEQ ID NO 62]
adaptor1(5) - AAT GAT ACG GCG ACC ACC GAG ATC TAC ACT CTT TCC CTA CAC
GAC GCT CTT CCG ATC TGC ATT [SEQ ID NO 63]
adaptor1(6) - AAT GAT ACG GCG ACC ACC GAG ATC TAC ACT CTT TCC CTA CAC
GAC GCT CTT CCG ATC TCA TGA [SEQ ID NO 64]
adaptor1(7) - AAT GAT ACG GCG ACC ACC GAG ATC TAC ACT CTT TCC CTA CAC
GAC GCT CTT CCG ATC TAT GCT [SEQ ID NO 65]
adaptor1(8) - AAT GAT ACG GCG ACC ACC GAG ATC TAC ACT CTT TCC CTA CAC
GAC GCT CTT CCG ATC TCT AGT [SEQ ID NO 66]
adaptor1(9) - AAT GAT ACG GCG ACC ACC GAG ATC TAC ACT CTT TCC CTA CAC
GAC GCT CTT CCG ATC TGC TAA [SEQ ID NO 67]
adaptor1(10) - AAT GAT ACG GCG ACC ACC GAG ATC TAC ACT CTT TCC CTA CAC
GAC GCT CTT CCG ATC TCA GTT [SEQ ID NO 68]
adaptor1(11) - AAT GAT ACG GCG ACC ACC GAG ATC TAC ACT CTT TCC CTA CAC
GAC GCT CTT CCG ATC TGT CAT [SEQ ID NO 69]
adaptor1(12) - AAT GAT ACG GCG ACC ACC GAG ATC TAC ACT CTT TCC CTA CAC
GAC GCT CTT CCG ATC TAC GTA [SEQ ID NO 70]
adaptor2(1) - ACA GTA GAT CGG AAG AGC GTC GTG TAG GGA AAG AGT GTA GAT
CTC GGT GG [SEQ ID NO 71]
adaptor2(2) TTC AGA GAT CGG AAG AGC GTC GTG TAG GGA AAG AGT GTA GAT
CTC GGT GG [SEQ ID NO 72]
adaptor2(3) AGT CAA GAT CGG AAG AGC GTC GTG TAG GGA AAG AGT GTA GAT
CTC GGT GG [SEQ ID NO 73]
adaptor2(4) TTG CAA GAT CGG AAG AGC GTC GTG TAG GGA AAG AGT GTA GAT
CTC GGT GG [SEQ ID NO 74]
adaptor2(5) AAT GCA GAT CGG AAG AGC GTC GTG TAG GGA AAG AGT GTA GAT
CTC GGT GG [SEQ ID NO 75]
adaptor2(6) TCA TGA GAT CGG AAG AGC GTC GTG TAG GGA AAG AGT GTA GAT
CTC GGT GG [SEQ ID NO 76]
adaptor2(7) AGC ATA GAT CGG AAG AGC GTC GTG TAG GGA AAG AGT GTA GAT
CTC GGT GG [SEQ ID NO 77]
adaptor2(8) ACT AGA GAT CGG AAG AGC GTC GTG TAG GGA AAG AGT GTA GAT
CTC GGT GG [SEQ ID NO 78]
adaptor2(9) TTA GCA GAT CGG AAG AGC GTC GTG TAG GGA AAG AGT GTA GAT
CTC GGT GG [SEQ ID NO 79]
adaptor2(10) AAC TGA GAT CGG AAG AGC GTC GTG TAG GGA AAG AGT GTA GAT
CTC GGT GG [SEQ ID NO 80]
adaptor2(11) ATG ACA GAT CGG AAG AGC GTC GTG TAG GGA AAG AGT GTA GAT
CTC GGT GG [SEQ ID NO 81]
adaptor2(12) TAC GTA GAT CGG AAG AGC GTC GTG TAG GGA AAG AGT GTA GAT
CTC GGT GG [SEQ ID NO 82]
PE2_short AAT GAT ACG GCG ACC ACC GA [SEQ ID NO 83]
WAS_sel PCR CAA GCA GAA GAC GGC ATA CGA GAT ACC TGC CGA GAA CAC A
[SEQ ID NO 84]
EMX1_sel PCR CAA GCA GAA GAC GGC ATA CGA GAT ACC TGC CGA GTC TTC T
[SEQ ID NO 85]
Lib adaptor 1 GAC GGC ATA CGA GAT [SEQ ID NO 86]
WAS lib adaptor 2 AAC AAT CTC GTA TGC CGT CTT CTG CTT G [SEQ ID NO 87]
EMX1 lib adaptor 2 TCT TAT CTC GTA TGC CGT CTT CTG CTT G [SEQ ID NO 88]
WAS lib seq PCR AAT GAT ACG GCG ACC ACC GAG ATC TAC ACT CTT TCC CTA
CAC GAC GCT CTT CCG ATC TNN NNA CCT ACC TGC CGA GAA CAC A [SEQ ID NO
89]
EMX1 lib seq PCR AAT GAT ACG GCG ACC ACC GAG ATC TAC ACT CTT TCC CTA
CAC GAC GCT CTT CCG ATC TNN NNA CCT ACC TGC CGA GTC TTC T [SEQ ID NO
90]
Lib fwd PCR CAA GCA GAA GAC GGC ATA CGA GAT [SEQ ID NO 91]
Modified CRISPR RNA Sequences
Unmodified crRNA
EMX1-RNA
rGrArGrUrCrCrGrArGrCrArGrArArGrArArGrArArGrUrUrUrUrArGrArGrCrUrArUrGrCrU
[SEQ ID NO 92]
BNA^{NC}-modified crRNA + N indicates BNA
EMX1-BNA. 1
rGrArG + UrCrCrGrArGrCrArGrArArGrArArGrArArGrUrUrUrUrArGrArGrCrUrArUrGrCrU
[SEQ ID NO 93]
EMX1-BNA. 2
rGrArG + U + CrCrGrArGrCrArGrArArGrArArGrArArGrUrUrUrUrArGrArGrCrUrArUrGrCrU
[SEQ ID NO 94]
EMX1-BNA. 3
rGrArG + U + C + CrGrArGrCrArGrArArGrArArGrArArGrUrUrUrUrArGrArGrCrUrArUrGrCrU
[SEQ ID NO 95]
EMX1-BNA. 4
+GrA + GrU + CrC + GrArGrCrArGrArArGrArArGrArArGrUrUrUrUrArGrArGrCrUrArUrGrCrU
[SEQ ID NO 96]
EMX1-BNA. 5
rGrArGrUrCrCrGrArGrC + ArGrArArGrArArGrArArGrUrUrUrUrArGrArGrCrUrArUrGrCrU
[SEQ ID NO 97]
EMX1-BNA. 6
rGrArGrUrCrCrGrArGrC + A + GrArArGrArArGrArArGrUrUrUrUrArGrArGrCrUrArUrGrCrU
[SEQ ID NO 98]
EMX1-BNA. 7
rGrArGrUrCrCrGrArGrC + A + G + ArArGrArArGrArArGrUrUrUrUrArGrArGrCrUrArUrGrCrU
[SEQ ID NO 99]
EMX1-BNA. 8
rGrArGrUrCrCrGrArGrCrA + G + A + ArGrArArGrArArGrUrUrUrUrArGrArGrCrUrArUrGrCrU
[SEQ ID NO 100]
EMX1-BNA. 9
rGrArGrUrCrCrG + ArG + CrA + GrA + ArGrArArGrArArGrUrUrUrUrArGrArGrCrUrArUrGrCrU
[SEQ ID NO 101]
EMX1-BNA. 10
rGrArGrUrCrCrGrArGrCrArGrArArGrA + ArGrArArGrUrUrUrUrArGrArGrCrUrArUrGrCrU
[SEQ ID NO 102]
EMX1-BNA. 11
rGrArGrUrCrCrGrArGrCrArGrArArGrA + A + GrArArGrUrUrUrUrArGrArGrCrUrArUrGrCrU
[SEQ ID NO 103]
EMX1-BNA. 12
rGrArGrUrCrCrGrArGrCrArGrArArGrA + A + G + ArArGrUrUrUrUrArGrArGrCrUrArUrGrCrU
[SEQ ID NO 104]
EMX1-BNA. 13
rGrArGrUrCrCrGrArGrCrArGrA + ArG + ArA + GrA + ArGrUrUrUrUrArGrArGrCrUrArUrGrCrU
[SEQ ID NO 105]
2′-O-methyl-modified crRNA (*N indicates 2′-O-methyl
EMX1-OME. 1
rGrArG*UrCrCrGrArGrCrArGrArArGrArArGrArArGrUrUrUrUrArGrArGrCrUrArUrGrCrU
[SEQ ID NO 106]
EMX1-OME. 2
rGrArG*U*CrCrGrArGrCrArGrArArGrArArGrArArGrUrUrUrUrArGrArGrCrUrArUrGrCrU
[SEQ ID NO 107]
EMX1-OME. 3
rGrArG*U*C*CrGrArGrCrArGrArArGrArArGrArArGrUrUrUrUrArGrArGrCrUrArUrGrCrU
[SEQ ID NO 108]
EMX1-OME. 4
*GrA*GrU*CrC*GrArGrCrArGrArArGrArArGrArArGrUrUrUrUrArGrArGrCrUrArUrGrCrU
[SEQ ID NO 109]
EMX1-OME. 5
rGrArGrUrCrCrGrArGrC*ArGrArArGrArArGrArArGrUrUrUrUrArGrArGrCrUrArUrGrCrU
[SEQ ID NO 110]
EMX1-OME. 6
rGrArGrUrCrCrGrArGrC*A*GrArArGrArArGrArArGrUrUrUrUrArGrArGrCrUrArUrGrCrU
[SEQ ID NO 111]
EMX1-OME. 7
rGrArGrUrCrCrGrArGrC*A*G*ArArGrArArGrArArGrUrUrUrUrArGrArGrCrUrArUrGrCrU
[SEQ ID NO 112]
EMX1-OME. 8
rGrArGrUrCrCrGrArGrCrA*G*A*ArGrArArGrArArGrUrUrUrUrArGrArGrCrUrArUrGrCrU
[SEQ ID NO 113]
EMX1-OME. 9
rGrArGrUrCrCrG*ArG*CrA*GrA*ArGrArArGrArArGrUrUrUrUrArGrArGrCrUrArUrGrCrU
[SEQ ID NO 114]
EMX1-OME. 10
rGrArGrUrCrCrGrArGrCrArGrArArGrA*ArGrArArGrUrUrUrUrArGrArGrCrUrArUrGrCrU
[SEQ ID NO 115]
EMX1-OME. 11
rGrArGrUrCrCrGrArGrCrArGrArArGrA*A*GrArArGrUrUrUrUrArGrArGrCrUrArUrGrCrU
[SEQ ID NO 116]
EMX1-OME. 12
rGrArGrUrCrCrGrArGrCrArGrArArGrA*A*G*ArArGrUrUrUrUrArGrArGrCrUrArUrGrCrU
[SEQ ID NO 117]
EMX1-OME. 13
rGrArGrUrCrCrGrArGrCrArGrA*ArG*ArA*GrA*ArGrUrUrUrUrArGrArGrCrUrArUrGrCrU
[SEQ ID NO 118]
2′-O-methyl-3′-phosphonoacetate modified crRNA (#N = 2′-O-methyl-3-
phosphonoacetate RNA)
EMX1-PAC. 1
rGrArG#UrCrCrGrArGrCrArGrArArGrArArGrArArGrUrUrUrUrArGrArGrCrUrArUrGrCrU
[SEQ ID NO 119]
EMX1-PAC. 2
rGrArG#U#CrCrGrArGrCrArGrArArGrArArGrArArGrUrUrUrUrArGrArGrCrUrArUrGrCrU
[SEQ ID NO 120]
EMX1-PAC. 3
rGrArG#U#C#CrGrArGrCrArGrArArGrArArGrArArGrUrUrUrUrArGrArGrCrUrArUrGrCrU
[SEQ ID NO 121]
EMX1-PAC. 4
#GrA#GrU#CrC#GrArGrCrArGrArArGrArArGrArArGrUrUrUrUrArGrArGrCrUrArUrGrCrU
[SEQ ID NO 122]
EMX1-PAC. 5
rGrArGrUrCrCrGrArGrC#ArGrArArGrArArGrArArGrUrUrUrUrArGrArGrCrUrArUrGrCrU
[SEQ ID NO 123]
EMX1-PAC. 6
rGrArGrUrCrCrGrArGrC#A#GrArArGrArArGrArArGrUrUrUrUrArGrArGrCrUrArUrGrCrU
[SEQ ID NO 124]
EMX1-PAC. 7
rGrArGrUrCrCrGrArGrC#A#G#ArArGrArArGrArArGrUrUrUrUrArGrArGrCrUrArUrGrCrU
[SEQ ID NO 125]
EMX1-PAC. 8
rGrArGrUrCrCrGrArGrCrA#G#A#ArGrArArGrArArGrUrUrUrUrArGrArGrCrUrArUrGrCrU
[SEQ ID NO 126]
EMX1-PAC. 9
rGrArGrUrCrCrG#ArG#CrA#GrA#ArGrArArGrArArGrUrUrUrUrArGrArGrCrUrArUrGrCrU
[SEQ ID NO 127]
EMX1-PAC. 10
rGrArGrUrCrCrGrArGrCrArGrArArGrA#ArGrArArGrUrUrUrUrArGrArGrCrUrArUrGrCrU
[SEQ ID NO 128]
EMX1-PAC. 11
rGrArGrUrCrCrGrArGrCrArGrArArGrA#A#GrArArGrUrUrUrUrArGrArGrCrUrArUrGrCrU
[SEQ ID NO 129]
EMX1-PAC. 12
rGrArGrUrCrCrGrArGrCrArGrArArGrA#A#G#ArArGrUrUrUrUrArGrArGrCrUrArUrGrCrU
[SEQ ID NO 130]
EMX1-PAC. 13
rGrArGrUrCrCrGrArGrCrArGrA#ArG#ArA#GrA#ArGrUrUrUrUrArGrArGrCrUrArUrGrCrU
[SEQ ID NO 131]
DNA-modified crRNA (dN indicates DNA)
EMX1-DNA. 1
dGdAdGdTdCdCdGdArGrCrArGrArArGrArArGrArArGrUrUrUrUrArGrArGrCrUrArUrGrCrU
[SEQ ID NO 132]
EMX1-DNA. 2
dGdAdGdTdCdCdGdAdGdCrArGrArArGrArArGrArArGrUrUrUrUrArGrArGrCrUrArUrGrCr
U [SEQ ID NO 133]
EMX1-DNA. 3
rGdAdGdTdCdCdGdAdGdCdAdGdAdArGrAdAdGrAdArGrUrUrUrUrArGrArGrCrUrArUrGr
CrU [SEQ ID NO 134]

This application includes a Sequence Listing which has been submitted in ASCII format and is hereby incorporated by reference in its entirety. Said ASCII copy was created on Nov. 2, 2018 and is named 21200720_1.txt, and has a size of 21 KB.

### Definitions and Interpretation

The description of the present invention has been presented for purposes of illustration and description, but it is not intended to be exhaustive or limited to the invention in the form disclosed. Many modifications and variations will be apparent to those of ordinary skill in the art without departing from the scope and spirit of the invention. Embodiments were chosen and described in order to best explain the principles of the invention and the practical application, and to enable others of ordinary skill in the art to understand the invention for various embodiments with various modifications as are suited to the particular use contemplated. To the extent that the following description is of a specific embodiment or a particular use of the invention, it is intended to be illustrative only, and not limiting of the claimed invention.

The corresponding structures, materials, acts, and equivalents of all means or steps plus function elements in the claims appended to this specification are intended to include any structure, material, or act for performing the function in combination with other claimed elements as specifically claimed.

References in the specification to “one embodiment”, “an embodiment”, etc., indicate that the embodiment described may include a particular aspect, feature, structure, or characteristic, but not every embodiment necessarily includes that aspect, feature, structure, or characteristic. Moreover, such phrases may, but do not necessarily, refer to the same embodiment referred to in other portions of the specification. Further, when a particular aspect, feature, structure, or characteristic is described in connection with an embodiment, it is within the knowledge of one skilled in the art to combine, affect or connect such aspect, feature, structure, or characteristic with other embodiments, whether or not such connection or combination is explicitly described. In other words, any element or feature may be combined with any other element or feature in different embodiments, unless there is an obvious or inherent incompatibility between the two, or it is specifically excluded.

It is further noted that the claims may be drafted to exclude any optional element. As such, this statement is intended to serve as antecedent basis for the use of exclusive terminology, such as “solely,” “only,” and the like, in connection with the recitation of claim elements or use of a “negative” limitation. The terms “preferably,” “preferred,” “prefer,” “optionally,” “may,” and similar terms are used to indicate that an item, condition or step being referred to is an optional (not required) feature of the invention.

The singular forms “a,” “an,” and “the” include the plural reference unless the context clearly dictates otherwise. The term “and/or” means any one of the items, any combination of the items, or all of the items with which this term is associated.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. A recited range (e.g., weight percents or carbon groups) includes each specific value, integer, decimal, or identity within the range and bounding the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc.

As will also be understood by one skilled in the art, all ranges described herein, and all language such as “up to”, “at least”, “greater than”, “less than”, “more than”, “or more”, and the like, include the number(s) recited and such terms refer to ranges that can be subsequently broken down into sub-ranges as discussed above.

### REFERENCES

All publications, patents and patent applications mentioned in this specification, and/or listed below, are indicative of the level of skill of those skilled in the art to which this invention pertains and are herein incorporated by reference to the same extent as if each individual publication, patent, or patent applications was specifically and individually indicated to be incorporated by reference.
Horvath P & Barrangou R (2010) CRISPR/Cas, the immune system of bacteria and archaea. Science 327(5962):167-170.
Jiang F & Doudna J A (2017) CRISPR-Cas9 Structures and Mechanisms. Annu Rev Biophys 46:505-529.
Sander J D & Joung J K (2014) CRISPR-Cas systems for editing, regulating and targeting genomes. Nat Biotechnol 32(4):347-355.
Joung J, et al. (2017) Genome-scale CRISPR-Cas9 knock out and transcriptional activation screening. Nat Protoc 12(4):828-863.
Cheong T C, Compagno M, & Chiarle R (2016) Editing of mouse and human immunoglobulin genes by CRISPR-Cas9 system. Nat Commun 7:10934.
Jinek M, et al. (2012) A programmable dual-RNA-guided DNA endonuclease inadaptive bacterial immunity. Science 337(6096):816-821.
Cong L, et al. (2013) Multiplex genome engineering using CRISPR/Cassystems. Science 339 (6121):819-823.
Jiang F, Zhou K, Ma L, Gressel S, & Doudna J A (2015) A Cas9-guide RNA complex pre organized for target DNA recognition. Science 348 (6242):1477-1481.
O'Geen H, Henry I M, Bhakta M S, Meckler J F, & Segal D J (2015) A genome-wide analysis of Cas9 binding specificity using ChIP-seq and targeted sequence capture. Nucleic Acids Res 43(6):3389-3404.
O'Geen H, Yu A S, & Segal D J (2015) How specific is CRISPR/Cas9 really? Curr Opin Chem Biol 29:72-78.
Xu X, Duan D, & Chen S J (2017) CRISPR-Cas9 cleavage efficiency correlates strongly with target-sg RNA folding stability: from physical mechanism to off-target assessment. Sci Rep 7(1):143.
Tycko J, Myer V E, & Hsu P D (2016) Methods for Optimizing CRISPR-Cas9 Genome Editing Specificity. Mol Cell 63(3):355-370.
Slaymaker I M, et al. (2016) Rationally engineered Cas9 nucleases with improved specificity. Science 351(6268):84-88.
Kleinstiver B P, et al. (2016) High-fidelity CRISPR-Cas9 nucleases with no detectable genome-wide off-target effects. Nature 529(7587):490-495.
Guilinger J P, Thompson D B, & Liu D R (2014) Fusion of catalytically inactive Cas9 to FokI nuclease improves the specificity of genome modification. Nat Biotechnol 32(6):577-582.
Zuris J A, et al. (2015) Cationic lipid-mediated delivery of proteins enables efficient protein-based genome editing in vitro and in vivo. Nat Biotechnol 33(1):73-80.
Fu Y, Sander J D, Reyon D, Cascio V M, & Joung J K (2014) Improving CRISPR-Cas nuclease specificity using truncated guide RNAs. Nat Biotechnol 32(3):279-284.
Hendel A, et al. (2015) Chemically modified guide RNAs enhance CRISPR-Cas genome editing in human primary cells. Nat Biotechnol 33(9):985-989.
Rahdar M, et al. (2015) Synthetic CRISPR RNA-Cas9-guided genome editing in human cells. Proc Natl Acad Sci USA 112(51):E7110-7117.

1. A method of increasing specificity of binding of a CRISPR-Cas protein to a target or off-target nucleic acid sequence, the method comprising:
contacting a target nucleic acid molecule comprising the target or off-target nucleic acid sequence with a complex comprising the CRISPR-Cas protein and a guide RNA, wherein the guide RNA comprises a complementarity region at the 5′ end of the guide RNA that binds to a complementary strand of the target nucleic acid sequence, wherein the guide RNA comprises at least one modified nucleic acid within the complementarity region, the at least one modified nucleic acid selected from the group consisting of a bridged nucleic acid, a deoxynucleic acid, and 2′-O-methyl RNA phosphonoacetate;
wherein the guide RNA complementarity region binds and directs the CRISPR-Cas protein to the target or off-target nucleic acid sequence.

2. The method of claim 1, wherein the selected target or off-target nucleic acid sequence is immediately 5′ of a protospacer adjacent motif (PAM).

3. The method of claim 1, wherein the at least one modified nucleic acid comprises a bridged nucleic acid.

4. The method of claim 3 wherein the bridged nucleic acid comprises a 2′,4′-bridged nucleic acid.

5. The method of claim 4, wherein the bridged nucleic acid is independently selected from:

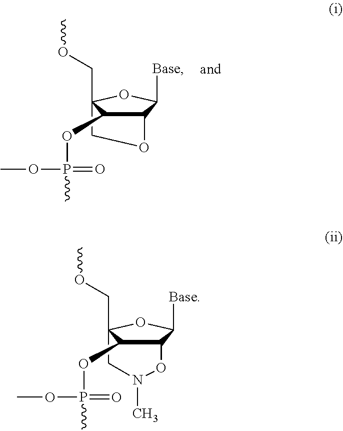

6. The method of claim 1, wherein the complementarity region at the 5′ end of the guide RNA comprises from about 16 to about 22 nucleic acids.

7. The method of claim 6, wherein the complementarity region at the 5′ end of the guide RNA comprises about 20 nucleic acids.

8. The method of claim 6, wherein the guide RNA comprises 3 or 4 modified nucleic acids located between positions 4 and 17 (inclusive) or between positions 14 and 20 (inclusive) from the 5′ end of the guide RNA.

9. The method of claim 8, wherein two, three or four of the modified nucleic acids are positioned adjacent to one another.

10. The method of claim 9, wherein two, three or four of the modified nucleic acids are each separated by a single non-modified nucleic acid.

11. The method of claim 8, wherein the modified nucleic acids are located between positions 10 and 15 (inclusive) from the 5′ end of the guide RNA.

12. The method of claim 8, wherein the modified nucleic acids are bridged nucleic acids, deoxynucleic acids, or 2′-O-methyl RNA phosphonoacetate nucleic acids.

13. (canceled)

14. (canceled)

15. The method of claim 1 wherein the target nucleic acid comprises an off-target nucleic acid sequence, wherein the off-target nucleic acid sequence comprises at least one nucleic acid that differs from the target nucleic acid sequence at a mismatch position, wherein the at least one modified nucleic acid is located on the guide RNA at a position corresponding or proximal to the mismatch position.

16. The method of claim 1, wherein the guide RNA comprises a (i) crRNA or a tracrRNA, (ii) a crRNA and a tracrRNA, or (iii) a single guide RNA.

17. The method of claim 1, wherein the CRISPR-Cas protein comprises a class 2 CRISPR-Cas protein.

18. The method of claim 17 wherein the class 2 CRISPR-Cas protein is selected from Cas9, dCas9, nCas9, Cpf1, C2c1, C2c2, and C2c3 proteins, and variants or homologs thereof.

19. (canceled)

20. (canceled)

21. (canceled)

22. The method of claim 18 wherein the class 2 CRISPR-Cas protein is an engineered variant of Cas9 which comprises eSpCas9, Cas9-HF1, or HypaCas9.

23. The method of claim 1, wherein the CRISPR-Cas protein is fused to an effector domain to form a fusion protein; optionally, wherein the CRISPR-Cas protein lacks nuclease activity, the fusion protein is functional, and/or the fusion protein serves as a marker.

24. (canceled)

25. (canceled)

26. The method of claim 23, wherein the effector domain is a transcriptional activator, a repressor, a DNA methyl transferase, a histone methyl/acetyl transferase, a histone deacetylase, an enzyme capable of modifying DNA or RNA (e.g. base editors), or a fluorescent or tagging protein.

27. The method of claim 1, wherein the CRISPR-Cas protein has nuclease activity, and the method increases specificity of cleavage of the selected target nucleic acid sequence by the CRISPR-Cas protein.

28. The method of claim 1, wherein the step of contacting the target nucleic acid molecule comprising the selected target or off-target nucleic acid sequence with the complex comprising the CRISPR-Cas protein and the guide RNA occurs in vitro or in vivo.

29. (canceled)

30. A guide RNA comprising a complementarity region at the 5′ end of the guide RNA that binds to a complementary strand of a target or off-target nucleic acid sequence, wherein the guide RNA comprises at least one modified nucleic acid within the complementarity region, the at least one modified nucleic acid selected from the group consisting of a bridged nucleic acid, a deoxynucleic acid, and 2′-O-methyl RNA phosphonoacetate, wherein the guide RNA complementarity region binds and directs a CRISPR-Cas protein to the target or off-target nucleic acid sequence.

31-45. (canceled)

46. A complex comprising a CRISPR-Cas protein and a guide RNA of claim 30.

47-58. (canceled)
